(19) United States
(12) Patent Application Publication
(10) Pub. No.: US 2003/0120049 A1
(43) Pub. Date: Jun. 26, 2003

(54) Non-selective cation channel

(76) Inventors: Guenter Schultz, Berlin (DE); Timothy Plant, Berlin (DE); Rainer Strotmann, Berlin (DE); Christian Harteneck, Berlin (DE); Karin Nunnenmacher, Berlin (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/812,143

(22) Filed: Mar. 19, 2001

Related U.S. Application Data
(60) Provisional application No. 60/201,036, filed on May 1, 2000.

(30) Foreign Application Priority Data
Mar. 17, 2000 (DE) DE 100 13 296.0

(51) Int. Cl. C07H 21/02; C07H 21/04

(52) U.S. Cl. 536/23.1

(57) ABSTRACT

The present invention relates to nucleic acids which code for the non-selective cation channel OTRPC4 as well as polypeptides which are coded by said nucleic acids. The invention further relates to hosts or host cells which express said polypeptide and processes for finding blockers, activators and modulators of said OTRPC4 cation channels. The invention includes blockers, activators and modulators of said OTRPC4 cation channels and pharmaceutical compositions containing said blockers, activators and modulators. In addition, the invention relates to non-human mammals which contain OTRPC4 either as a transgene, an inactivated gene (knock-out) or modified gene (knock-in).

### RELATED APPLICATION

The benefit of prior provisional application Serial No. 60,201,036, filed May 5, 2000 is hereby claimed.

### BACKGROUND OF THE INVENTION

Cells are exposed to different extracellular ion concentrations and hence different osmolarities depending on the physiological state of the tissue to which they belong. A reduction in the extracellular osmolarity leads to an increase in extracellular volume as a result of an influx of extracellular fluid. This increase in volume threatens the homeostasis of the cell so that evolution developed a mechanism, the activation of which enables a cell to actively counter-regulate the osmotically induced increase in volume. This mechanism is referred to as “regulated volume decrease” (RVD) (for a summary see Ref. 1). The molecular mechanism on which RVD is based is not known at present but various studies have shown a transient rise in the intracellular calcium concentrations which accompanied the volume regulation and could be inhibited using lanthanum and gadolinium. Thus, it is possible that a non-selective calcium-permeable channel is involved in RVD.

In C. elegans, a cDNA was cloned which codes for a channel having an affinity for the TRP (“transient receptor potential”) family of non-selective cation channels. This channel is responsible for the reactions of C. elegans to solutions of higher osmolarity and is therefore referred to as OSM-9 (2). However, at present, nothing is known of the biophysical characteristics of OSM-9 and no corresponding homologous protein has hitherto been described for mammals.

The family of TRP channels (TRPCs) (3) can be divided into three different subfamilies (4). The biggest family is the STRP subfamily (short TRP; named after its short N-terminus), consisting of the classic drosophila channels TRP and TRPL (transient receptor potential-like) (5) and 7 mammalian homologs of TRP (TRPC1-7) (6-15). The channels in this family are involved in the calcium influx which is initiated by the activation of receptors which share the common feature of activating phospholipase C. The second subfamily of the TRPCs was named OTRPC, after the first member of this family OSM-9. The channels of this family are activated by chemical and physical stimulation. The OTRPC family includes the vanilloid receptor (VR1) (16, 17), the vanilloid-like receptor (VRL-1, also known as GRC) (18, 19), and a channel which may possibly function as an epithelial calcium channel (known as EcaC or also CaT1) (20, 21). VR1 is a non-selective calcium-permeable channel which was cloned from the dorsal ganglion cells of rats (16). This channel is activated by heat and by the substance capsaicin which is a pain trigger. The recently cloned channel related to VR1, namely VRL-1, can be activated by heat and might be involved in pain reception (18). In any case, its widespread expression could also be an indication that this channel has other functions, e.g. it has recently been shown that this channel is involved in the intracellular transport of “insulin-like growth factor 1” (IGF-1) (19). Other members of this OTRPC family are EcaC, which was cloned from rabbit kidneys (20) and CaT1 (21) which was cloned from rat duodenum; the two channels are identical in sequence and are involved in the vitamin D-induced influx of calcium in epithelial cells (20, 21). The third TRP subfamily is known as LTRPC (long TRP channels, named after their long N-terminus) and hitherto consists of the two substances melastatin (22) and TRPC7 (Foskett, J. K. in Cellular and Molecular Physiology of Cell Volume Regulation (ed. Strange, K.) 259-277 (CRC Press, Boca Raton, 1994).

International Patent Application WO 00/32766 discloses human vanilloid receptors and their use.

The aim of the present invention is to provide a new TRP channel with advantageous properties compared with the channels known from the prior art described above.

### SUMMARY OF THE INVENTION

The present invention relates to nucleic acids which code for the non-selective cation channel OTRPC4 as well as polypeptides which are coded by said nucleic acids. The invention further relates to hosts or host cells which express the said polypeptide and methods for finding blockers, activators and modulators of said OTRPC4 cation channels. The invention includes blockers, activators and modulators of said OTRPC4 cation channels as well as pharmaceutical compositions containing said blockers, activators and modulators. The invention also relates to non-human mammals which contain either OTRPC4 as a transgene, inactivated gene (knock-out) or modified gene (knock-in).

### DESCRIPTION OF THE FIGURES

FIG. 1: Amino acid sequence of the predicted pore-forming structure of OTRPC4 and tissue distribution of the expression of OTRPC4.

(a) The Figure shows the amino acid sequence of the predicted fifth and sixth transmembranal domains and the adjacent cytosolic domain of OTRPC4. The transmembranal region 5 and 6 and the presumed pore-forming cytoplasmatic domain are marked as such and the conserved amino acids are deposited. (b) Autoradiogram of a Northern-blot of different mouse tissues using the EST-sequences of mouse cDNA coding for OTRPC4 as probe. A 3.3 kb fragment is detected in the RNA of heart, liver, kidney and testis, while an additional 2.2 kb fragment can be detected in the RNA of liver and kidney.

FIG. 2: Sequences of cDNA coding for OTRPC4 of the mouse and organisation of the genomic clone of OTRPC4. The translation start and stop codons and the transitions between exons and introns and the length of the introns are marked. Under the DNA-sequence, the amino acid sequence is shown, the predicted transmembranal regions and the ankyrin binding site are marked.

FIG. 3: In situ hybridisation of murine kidney and brain for detecting the expression of OTRPC4.

The Figure shows a sagittal section (a) and a horizontal section (f) through a whole mouse kidney, two enlargements of the sagittal section of the kidney (b, c), a sagittal section (e), a coronary section (f), a horizontal section (g) of a whole mouse brain and an enlargement of the sagittal section of a mouse brain (h). The sections were prepared, after fixing of the tissue, using a microtome and then hybridised with a radiolabelled RNA-probe of the coding region of murine-OTRPC4. The Figure shows the expression of OTRPC4 in the distal convoluted renal tubule (b, c) and in the choroid plexus of the brain ventricle (h).

FIG. 4: Increase in the intracellular calcium concentration in HEK293 cells transfected with a plasmid which expresses the cDNA of OTRPC4. The intracellular calcium concentration was measured using the FURA-2 technique in cells which express OTRPC4 and compared with cells which do not express this channel. The cells were initially cultivated in isotonic solution containing 100 mM mannitol and 1 mM CaCl₂. The upper horizontal bar indicates the change from the extracellular solution washing around the cells to a 200 mM solution. The change in osmolarity was achieved by omitting the mannitol.

In the space of time indicated by the lower horizontal bar, the calcium in the extracellular solution was replaced by EGTA. The traces shown represent the averages of 17 cells (for the OTRPC4-expressing cells) and 21 cells (for the controll cells) in the same experiment.

The small figure indicates the corresponding measuring traces for individal OTRPC4-expressing cells in the same experiment.

FIG. 5: Osmolarity-dependent change in the intracellular calcium concentration in HEK293 cells which transiently express OTRPC4. The Figure shows the maximum fluorescence quotient of the calcium-charged and -uncharged FURA-2 stain dependent on the osmolarity of extracellular solution.

FIG. 6: Reduction in the ion flux in OTRPC4-expressing cells in a hyperosmolar extracellular solution. The ion flux is recorded by voltage ramps from −100 to +100 mM in a standard extracellular solution (osmolarity 305 mosmol/l; 1) and after the addition of a mannitol-containing solution with an osmolarity of 320 mosmol/l (2). The small Figure shows the progress of the effect over time triggered by increasing the osmolarity of the extracellular solution.

FIG. 7: Increase in ion flux carried by cations triggered by hypotonic extracellular solution in cells which express OTRPC4. (A) The whole cell ion flux of an OTRPC4-expressing cell was measured at −100 and +100 mV. At the time indicated by the horizontal bar, the extracellular standard solution was replaced by a solution containing 100 mM NaCl and 100 mM mannitol (osmolarity 320 mosmol/l), then replaced by a solution without mannitol (215 mosmol/l) and then again replaced by a hypoosmolar solution in which sodium and calcium had been replaced by NMDG. Finally, the cell was rinsed again with 320 milliosmolar solution. (B) This shows the ion flux which was triggered by an individual voltage ramp in an OTRPC4-expressing cell at the times indicated by numbers in (A).

FIG. 8: Western Blot of OTRPC4 in subcellular fractions. The figure shows that ORTPC4 is a protein residing in the cell membrane.

FIG. 9: Calcium dependent fluorescence changes on Fura-2 loaded Plexus choroideus cells.

FIG. 10: Measurement of OTRPC4 mediated change of intracellular calcium concentration in HEK293 cells expressing OTRPC4 channel.

### DESCRIPTION OF THE INVENTION

The objective has been achieved within the scope of the claims and specification of the present invention.

The use of the single or plural in the claims or specification should in no way be regarded as limiting and should also include the other form. RNA means the same as RNS and DNA the same as DNS.

The invention relates to a nucleic acid, characterised in that it codes for the non-selective cation channel OTRPC4 or for a fragment, a functional variant, an allelic variant or a subunit, or variants of said nucleic acid on the basis of the degenerative code or a nucleic acid which is able to hybridise with said nucleic acid. The cation channel according to the invention or OTRPC4 polypeptides are described in more detail hereinafter. OTRPC4 nucleic acids according to the invention are preferably eukaryotic nucleic acids, most preferably human or murine but may also be derived from the rat, hamster, goat, cattle, pigs, sheep, dogs, cats, monkeys and other eukaryotes known in the art. For example, the said nucleic acid may be a recombinantly produced nucleic acid, e.g. a cDNA. Nucleic acids according to the invention are shown by way of example in the Figures and in the Example.

A nucleic acid RNA is preferred according to the invention. Also preferred is a nucleic acid DNA according to the invention.

Also preferred is a nucleic acid according to the invention characterised in that it contains 5′ or 3′ or 5′ and 3′ untranslated regions. The nucleic acid according to the invention may have further untranslated regions upstream and/or downstream. The said untranslated region may comprise a regulatory element such as a transcription initiation unit (promoter) or enhancer. The said promoter may be, for example, a constitutively active or inducible or development-controlled promoter. The constitutive promoters of human cytomegalovirus (CMV) and Rous sarcoma virus (RSV) as well as Simian virus 40 (SV40) and Herpes simplex virus (HSV) promoter are preferred, without ruling out other known promoters. Inducible promoters according to the invention comprise antibiotic-resistant promoters, heat shock promoters, hormone-inducible “Mouse Mammary Tumour Virus” (MMTV) promoter and metallothioneine promoter.

Also preferred is a nucleic acid according to the invention, characterised in that it codes for a fragment of the non-selective cation channel OTRPC4.

Also preferred is a nucleic acid according to the invention, characterised in that it codes for a functional variant of the non-selective cation channel OTRPC4.

Also preferred is a nucleic acid according to the invention, characterised in that it codes for an allelic variant of the non-selective cation channel OTRPC4.

Also preferred is a nucleic acid according to the invention, characterised in that it codes for variants of nucleic acid on the basis of the degenerative code.

Also preferred is a nucleic acid, characterised in that it is capable of hybridising with a nucleic acid according to the invention under stringent conditions. Stringent conditions are known to the skilled person and can be found in particular in Sambrook et al. (1989).

Molecular Cloning: A Laboratory Manual, 2nd Ed., Cold Spring Harbor Laboratory Press, Cold Spring Harbor, N.Y.

Also preferred is a nucleic acid according to the invention, characterised in that the said non-selective cation channel OTRPC4 is a mammalian cation channel.

Also preferred is a nucleic acid according to the invention, characterised in that the said non-selective cation channel OTRPC4 is murine.

Also preferred is a nucleic acid according to the invention, characterised in that the said non-selective cation channel OTRPC4 is human.

Also preferred is a nucleic acid which is characterised in that it contains the sequence

| | |
|---|---|
| CTCTCACCGCCTACTACCAGCCGCTGGAGGGCACAATGGCGGATTCCAGCGAAGGCCC | (SEQ ID NO:_) |
| CCGCGCGGGGCCCGGGGAGGTGGCTGAGCTCCCCGGGGATGAGAGTGGCACCCCAGG | |
| TGGGGAGGCTTTTCCTCTCTCCTCCCTGGCCAATCTGTTTGAGGGGGAGGATGGCTCCC | |
| TTTCGCCCTCACCGGCTGATGCCAGTCGCCCTGCTGGCCCAGGCGATGGGCGACCAAA | |
| TCTGCGCATGAAGTTCCAGGGCGCCTTCCGCAAGGGGGTGCCCAACCCCATCGATCTG | |
| CTGGAGTCCACCCTATATGAGTCCTCGGTGGTGCCTGGGCCCAAGAAGCACCCATGG | |
| ACTCACTGTTTGACTACGGCACCTATCGTCACCACTCCAGTGACAACAAGAGGTGGAG | |
| GAAGAAGATCATAGAGAAGCAGCCGCAGAGCCCCAAAGCCCCTGCCCCTCAGCCGCC | |
| CCCCATCCTCAAAGTCTTCAAGCGGCCTATCCTCTTTGACATCGTGTCCCGGGGCTCCA | |
| CTGCTGACCTGGACGGGCTGCTCCCATTCTTGCTGACCCACAAGAAACGCCTAACTGA | |
| TGAGGAGTTTCGAGAGCCATCTACGGGGAAGACCTGCCTGCCCAAGGCCTTGCTGAAC | |
| CTGAGCAATGGCCGCAACGACACCATCCCTGTGCTGCTGGACATCGCGGAGCGCACCG | |
| GCAACATGCGGGAGTTCATTAACTCGCCCTTCCGTGACATCTACTATCGAGGTCAGAC | |
| AGCCCTGCACATCGCCATTGAGCGTCGCTGCAAACACTACGTGGAACTTCTCGTGGCC | |
| CAGGGAGCTGATGTCCACGCCCAGGCCCGTGGGCGCTTCTTCCAGCCCAAGGATGAGG | |
| GGGGCTACTTCTACTTTGGGGAGCTGCCCCTGTCGCTGGCTGCCTGCACCAACCAGCCC | |
| CACATTGTCAACTACCTGACGGAGAACCCCCACAAGAAGGCGGACATGCGGCGCCAG | |
| GACTCGCGAGGCAACACAGTGCTGCATGCGCTGGTGGCCATTGCTGACAACACCCGTG | |
| AGAACACCAAGTTTGTTACCAAGATGTACGACCTGCTGCTGCTCAAGTGTGCCCGCCT | |
| CTTCCCCGACAGCAACCTGGAGGCCGTGCTCAACAACGACGGCCTCTCGCCCCTCATG | |
| ATGGCTGCCAAGACGGGCAAGATTGGGATCTTTCAGCACATCATCCGGCGGGAGGTGA | |
| CGGATGAGGACACACGGCACCTGTCCCGCAAGTTCAAGGACTGGGCCTATGGGCCAGT | |
| GTATTCCTCGCTTTATGACCTCTCCTCCCTGGACACGTGTGGGGAAGAGGCCTCCGTGC | |
| TGGAGATCCTGGTGTACAACAGCAAGATTGAGAACCGCCACGAGATGCTGGCTGTGGA | |
| GCCCATCAATGAACTGCTGCGGGACAAGTGGCGCAAGTTCGGGGCCGTCTCCTTCTAC | |
| ATCAACGTGGTCTCCTACCTGTGTGCCATGGTCATCTTCACTCTCACCGCCTACTACCA | |
| GCCGCTGGAGGGCACACCGCCGTACCCTTACCGCACCACGGTGGACTACCTGCGGCTG | |
| GCTGGCGAGGTCATTACGCTCTTCACTGGGGTCCTGTTCTTCTTCACCAACATCAAAGA | |
| CTTGTTCATGAAGAAATGCCCTGGAGTGAATTCTCTCTTCATTGATGGCTCCTTCCAGC | |
| TGCTCTACTTCATCTACTCTGTCCTGGTGATCGTCTCAGCAGCCCTCTACCTGGCAGGG | |
| ATCGAGGCCTACCTGGCCGTGATGGTCTTTGCCCTGGTCCTGGGCTGGATGAATGCCCT | |
| TTACTTCACCCGTGGGCTGAAGCTGACGGGGACCTATAGCATCATGATCCAGAAGATT | |
| CTCTTCAAGGACCTTTTCCGATTCCTGCTCGTCTACTTGCTCTTCATGATCGGCTACGCT | |
| TCAGCCCTGGTCTCCCTCCTGAACCCGTGTGCCAACATGAAGGTGTGCAATGAGGACC | |
| AGACCAACTGCACAGTGCCCACTTACCCCTCGTGCCGTGACAGCGAGACCTTCAGCAC | |
| CTTCCTCCTGGACCTGTTTAAGCTGACCATCGGCATGGGCGACCTGGAGATGCTGAGC | |
| AGCACGAAGTACCCCGTGGTCTTCATCATCCTGCTGGTGACCTACATCATCCTCACCTT | |
| TGTGCTGCTCCTCAACATGCTCATTGCCCTCATGGGCGAGACAGTGGGCCAGGTCTCCA | |
| AGGAGAGCAAGCACATCTGGAAGCTGCAGTGGGCCACCACCATCCTGGACATTGAGC | |
| GCTCCTTCCCCGTATTCCTGAGGAAGGCCTTCCGCTCTGGGGAGATGGTCACCGTGGGC | |
| AAGAGCTCGGACGGCACTCCTGACCGCAGGTGGTGCTTCAGGGTGGATGAGGTGAACT | |
| GGTCTCACTGGAACCAGAACTTGGGCATCATCAACGAGGACCCGGGCAAGAATGAGA | |
| CCTACCAGTATTATGGCTTCTCGCATACCGTGGGCCGCCTCCGCAGGGATCGCTGGTCC | |
| TCGGTGGTACCCCGCGTGGTGGAACTGAACAAGAACTCGAACCCGGACGAGGTGGTG | |
| GTGCCTCTGGACAGCATGGGGAACCCCCGCTGCGATGGCCACCAGCAGGGTTACCCCC | |
| GCAAGTGGAGGACTGAGGACGCCCCGCTCTAGGGACTGCAGCCCAGCCCCAGCTTCTC | |
| TGCCCACTCATTTCTAGTCCAGCCGCATTTCAGCAGTGCCTTCTGGGGTGTCCCCCCAC | |
| ACCCTGCTTTGGCCCCAGAGGCGAGGGACCAGTGGAGGTGCCAGGGAGGCCCCAGGA | |
| CCCTGTGGTCCCCTGGCTCTGCCTCCCCACCCTGGGGTGGGGGCTCCCGGCCACCTGTC | |
| TTGCTCCTATGGAGTCACATAAGCCAACGCCAGAGCCCCTCCACCTCAGGCCCCAGCC | |
| CCTGCCTCTCCATTATTTATTTGCTCTGCTCTCAGGAAGCGACGTGACCCCTGCCCCAG | |
| CTGGAACCTGGCAGAGGCCTTAGGACCCCGTTCCAAGTGCACTGCCCGGCCAAGCCCC | |
| AGCCTCAGCCTGCGCCTGAGCTGCATGCGCCACCATTTTTGGCAGCGTGGCAGCTTTGC | |
| AAGGGGCTGGGGCCCTCGGCGTGGGGCCATGCCTTCTGTGTGTTCTGTAGTGTCTGGG | |
| ATTTGCCGGTGCTCAATAAATGTTTATTCATTGACGGTGAAAAAAAAAAAAAAAAAAA, | |

or a partial sequence thereof, a nucleic acid which is capable of hybridising with said sequence under stringent conditions, an allelic variant or a functional variant of said sequence or a variant of nucleic acid on the basis of the degenerative code. According to the invention, the sequence shown above also comprises the human OTRPC4 DNA sequence with 5′ and 3′-untranslated sequences.

The nucleic acids according to the invention are given in accordance with the Internationally recognised IUPAC nomenclature, i.e. the letter R denotes an A or G, M denotes an A or C, S denotes a C or G, Y denotes a C or T, K denotes a G or T and W denotes an A or T.

Also preferred is a nucleic acid which is characterised in that it has the sequence

| | |
|---|---|
| CTCTCACCGCCTACTACCAGCCGCTGGAGGGCACAATGGCGGATTCCAGCGAAGGCCC | (SEQ ID NO:_) |
| CCGCGCGGGGCCCGGGGAGGTGGCTGAGCTCCCCGGGGATGAGAGTGGCACCCCAGG | |
| TGGGGAGGCTTTTCCTCTCTCCTCCCTGGCCAATCTGTTTGAGGGGGAGGATGGCTCCC | |
| TTTCGCCCTCACCGGCTGATGCCAGTCGCCCTGCTGGCCCAGGCGATGGGCGACCAAA | |
| TCTGCGCATGAAGTTCCAGGGCGCCTTCCGCAAGGGGGTGCCCAACCCCATCGATCTG | |
| CTGGAGTCCACCCTATATGAGTCCTCGGTGGTGCCTGGGCCCAAGAAAGCACCCATGG | |
| ACTCACTGTTTGACTACGGCACCTATCGTCACCACTCCAGTGACAACAAGAGGTGGAG | |
| GAAGAAGATCATAGAGAAGCAGCCGCAGAGCCCCAAAGCCCCTGCCCCTCAGCCGCC | |
| CCCCATCCTCAAAGTCTTCAACCGGCCTATCCTCTTTGACATCGTGTCCCGGGGCTCCA | |
| CTGCTGACCTGGACGGGCTGCTCCCATTCTTGCTGACCCACAAGAAACGCCTAACTGA | |
| TGAGGAGTTTCGAGAGCCATCTACGGGGAAGACCTGCCTGCCCAAGGCCTTGCTGAAC | |
| CTGAGCAATGGCCGCAACGACACCATCCCTGTGCTGCTGGACATCGCGGAGCGCACCG | |
| GCAACATGCGGGAGTTCATTAACTCGCCCTTCCGTGACATCTACTATCGAGGTCAGAC | |
| AGCCCTGCACATCGCCATTGAGCGTCGCTGCAAACACTACGTGGAACTTCTCGTGGCC | |
| CAGGGAGCTGATGTCCACGCCCAGGCCCGTGGGCGCTTCTTCCAGCCCAAGGATGAGG | |
| GGGGCTACTTCTACTTTGGGGAGCTGCCCCTGTCGCTGGCTGCCTGCACCAACCAGCCC | |
| CACATTGTCAACTACCTGACGGAGAACCCCCACAAGAAGGCGGACATGCGGCGCCAG | |
| GACTCGCGAGGCAACACAGTGCTGCATGCGCTGGTGGCCATTGCTGACAACACCCGTG | |
| AGAACACCAAGTTTGTTACCAAGATGTACGACCTGCTGCTGCTCAAGTGTGCCCGCCT | |
| CTTCCCCGACAGCAACCTGGAGGCCGTGCTCAACAACGACGGCCTCTCGCCCCTCATG | |
| ATGGCTGCCAAGACGGGCAAGATTGGGATCTTTCAGCACATCATCCGGCGGGAGGTGA | |
| CGGATGAGGACACACGGCACCTGTCCCGCAAGTTCAAGGACTGGGCCTATGGGCCAGT | |
| GTATTCCTCGCTTTATGACCTCTCCTCCCTGGACACGTGTGGGGAAGAGGCCTCCGTGC | |
| TGGAGATCCTGGTGTACAACAGCAAGATTGAGAACCGCCACGAGATGCTGGCTGTGGA | |
| GCCCATCAATGAACTGCTGCGGGACAAGTGGCGCAAGTTCGGGGCCGTCTCCTTCTAC | |
| ATCAACGTGGTCTCCTACCTGTGTGCCATGGTCATCTTCACTCTCACCGCCTACTACCA | |
| GCCGCTGGAGGGCACACCGCCGTACCCTTACCGCACCACGGTGGACTACCTGCGGCTG | |
| GCTGGCGAGGTCATTACGCTCTTCACTGGGGTCCTGTTCTTCTTCACCAACATCAAAGA | |
| CTTGTTCATGAAGAAATGCCCTGGAGTGAATTCTCTCTTCATTGATGGCTCCTTCCAGC | |
| TGCTCTACTTCATCTACTCTGTCCTGGTGATCGTCTCAGCAGCCCTCTACCTGGCAGGG | |
| ATCGAGGCCTACCTGGCCGTGATGGTCTTTGCCCTGGTCCTGGGCTGGATGAATGCCCT | |
| TTACTTCACCCGTGGGCTGAAGCTGACGGGGACCTATAGCATCATGATCCAGAAGATT | |
| CTCTTCAAGGACCTTTTCCGATTCCTGCTCGTCTACTTGCTCTTCATGATCGGCTACGCT | |
| TCAGCCCTGGTCTCCCTCCTGAACCCGTGTGCCAACATGAAGGTGTGCAATGAGGACC | |
| AGACCAACTGCACAGTGCCCACTTACCCCTCGTGCCGTGACAGCGAGACCTTCAGCAC | |
| CTTCCTCCTGGACCTGTTTAAGCTGACCATCGGCATGGGCGACCTGGAGATGCTGAGC | |
| AGCACCAAGTACCCCGTGGTCTTCATCATCCTGCTGGTGACCTACATCATCCTCACCTT | |
| TGTGCTGCTCCTCAACATGCTCATTGCCCTCATGGGCGAGACAGTGGGCCAGGTCTCCA | |
| AGGAGAGCAAGCACATCTGGAAGCTGCAGTGGGCCACCACCATCCTGGACATTGAGC | |
| GCTCCTTCCCCGTATTCCTGAGGAAGGCCTTCCGCTCTGGGGAGATGGTCAGCGTGGGC | |
| AAGAGCTCGGACGGCACTCCTGACCGCAGGTGGTGCTTCAGGGTGGATGAGGTGAACT | |
| GGTCTCACTGGAACCAGAACTTGGGCATCATCAACGAGGACCCGGGCAAGAATGAGA | |
| CCTACCAGTATTATGGCTTCTCGCATACCGTGGGCCGCCTCCGCAGGGATCGCTGGTCC | |
| TCGGTGGTACCCCGCGTGGTGGAACTGAACAAGAACTCGAACCCGGACGAGGTGGTG | |
| GTGCCTCTGGACAGCATGGGGAACCCCCGCTGCGATGGCCACCAGCAGGGTTACCCCC | |
| GCAAGTGGAGGACTGAGGACGCCCCGCTCTAGGGACTGCAGCCCAGCCCCAGCTTCTC | |
| TGCCCACTCATTTCTAGTCCAGCCGCATTTCAGCAGTGCCTTCTGGGGTGTCCCCCCAC | |
| ACCCTGCTTTGGCGCCAGAGGCGAGGGACCAGTGGAGGTGCCAGGGAGGCCCCAGGA | |
| CCCTGTGGTCCCCTGGCTCTGCCTCCCCACCCTGGGGTGGGGGCTCCCGGCCACCTGTC | |
| TTGCTCCTATGGAGTCACATAAGCCAACGCCAGAGCCCCTCCACCTCAGGCCCCAGCC | |
| CCTGCCTCTCCATTATTTATTTGCTCTGCTCTCAGGAAGCGACGTGACCCCTGCCCCAG | |
| CTGGAACCTGGCAGAGGCCTTAGGACCCCGTTCCAAGTGCACTGCCCGGCCAAGCCCC | |
| AGCCTCAGCCTGCGCCTGAGCTGCATGCGCCACCATTTTTGGCAGCGTGGCAGCTTTGC | |
| AAGGGGCTGGGGCCCTCGGCGTGGGGCGATGCCTTCTGTGTGTTCTGTAGTGTCTGGG | |
| ATTTGCCGGTGCTCAATAAATGTTTATTCATTGACGGTGAAAAAAAAAAAAAAAAAAA. | |

The sequence above is, according to the invention, the human OTRPC4 DNA sequence with 5′ and 3′-untranslated sequences.

Also preferred is a nucleic acid which is characterised in that it contains the sequence

| | |
|---|---|
| ATGGCGGATTCCAGCGAAGGCCCCCGCGCGGGGCCCGGGGAGGTGGCTGAGCTCCCC | (SEQ ID NO:_) |
| GGGGATGAGAGTGGCACCCCAGGTGGGGAGGCTTTTCCTCTCTCCTCCCTGGCCAATC | |
| TGTTTGAGGGGGAGGATGGCTCCCTTTCGCCCTCACCGGCTGATGCCAGTCGCCCTGCT | |
| GGCCCAGGCGATGGGCGACCAAATCTGCGCATGAAGTTCCAGGGCGCCTTCCGCAAGG | |
| GGGTGCCCAACCCCATCGATCTGCTGGAGTCCACCCTATATGAGTCCTCGGTGGTGCCT | |
| GGGCCCAAGAAAGCAGCCATGGACTCACTGTTTGACTACGGCACCTATCGTCACCACT | |
| CCAGTGACAACAAGAGGTGGAGGAAGAAGATCATAGAGAAGCAGCCGCAGAGCCCCA | |
| AAGCCCCTGCCCCTCAGCCGCCCCCCATCCTCAAAGTCTTCAACCGGCCTATCCTCTTT | |
| GACATCGTGTCCCGGGGCTCCACTGCTGACCTGGACGGGCTGCTCCCATTCTTGCTGAC | |
| CCACAAGAAACGCCTAACTGATGAGGAGTTTCGAGAGCCATCTACGGGGAAGACCTG | |
| CCTGCCCAAGGCCTTGCTGAACCTGAGCAATGGCCGCAACGACACCATCCCTGTGCTG | |
| CTGGACATCGCGGAGCGCACCGGCAACATGCGGGAGTTCATTAACTCGCCCTTCCGTG | |
| ACATCTACTATCGAGGTCAGACAGCCCTGCACATCGCCATTGAGCGTCGCTGCAAACA | |
| CTACGTGGAACTTCTCGTGGCCCAGGGAGCTGATGTCCACGCCCAGGCCCGTGGGCGC | |
| TTCTTCCAGCCCAAGGATGAGGGGGGCTACTTCTACTTTGGGGAGCTGCCCCTGTCGCT | |
| GGCTGCCTGCACCAACCAGCCCCACATTGTCAACTACCTGACGGAGAACCCCCACAAG | |
| AAGGCGGACATGCGGCGCCAGGACTCGCGAGGCAACACAGTGCTGCATGCGCTGGTG | |
| GCCATTGCTGACAACACCCGTGAGAACACCAAGTTTGTTACCAAGATGTACGACCTGC | |
| TGCTGCTCAAGTGTGCCCGCCTCTTCCCCGACAGCAACCTGGAGGCCGTGCTCAACAA | |
| CGACGGCCTCTCGCCCCTCATGATGGCTGCCAAGACGGGCAAGATTGGGATCTTTCAG | |
| CACATCATCCGGCGGGAGGTGACGGATGAGGACACACGGCACCTGTCCCGCAAGTTCA | |
| AGGACTGGGCCTATGGGCCAGTGTATTCCTCGCTTTATGACCTCTCCTCCCTGGACACG | |
| TGTGGGGAAGAGGCCTCCGTGCTGGAGATCCTGGTGTACAACAGCAAGATTGAGAACC | |
| GCCACGAGATGCTGGCTGTGGAGCGCATCAATGAACTGCTGCGGGACAAGTGGCGCA | |
| AGTTCGGGGCCGTCTCCTTCTACATCAACGTGGTCTCCTACCTGTGTGCCATGGTCATC | |
| TTCACTCTCACCGCCTACTACGAGGCGCTGGAGGGCACACCGCCGTACCCTTACCGCAC | |
| CACGGTGGACTACCTGCGGCTGGCTGGCGAGGTCATTACGCTCTTCACTGGGGTCCTGT | |
| TCTTCTTCACCAACATCAAAGACTTGTTCATGAAGAAATGCCCTGGAGTGAATTCTCTC | |
| TTCATTGATGGCTCCTTCCAGCTGCTCTACTTCATCTACTCTGTCCTGGTGATCGTCTCA | |
| GCAGCCCTCTACCTGGCAGGGATCGAGGCCTACCTGGCCGTGATGGTCTTTGCCCTGGT | |
| CCTGGGCTGGATGAATGCCCTTTACTTCACCCGTGGGCTGAAGCTGACGGGGACCTAT | |
| AGCATCATGATCCAGAAGATTCTCTTCAAGGACCTTTTCCGATTCCTGCTCGTCTACTT | |
| GCTCTTCATGATCGGCTACGCTTCAGCCCTGGTCTCCCTCCTGAACCCGTGTGCCAACA | |
| TGAAGGTGTGCAATGAGGACCAGACCAACTGCACAGTGCCCACTTACCCCTCGTGCCG | |
| TGACAGCGAGACCTTCAGCACCTTCCTCCTGGACCTGTTTAAGCTGACCATCGGCATGG | |
| GCGACCTGGAGATGCTGAGCAGCACCAAGTACCCCGTGGTCTTGATCATCCTGCTGGT | |
| GACCTACATCATCCTCACCTTTGTGCTGCTCCTCAACATGCTCATTGCCCTCATGGGCG | |
| AGACAGTGGGCCAGGTCTCCAAGGAGAGCAAGCACATCTGGAAGCTGCAGTGGGCCA | |
| CCACCATCCTGGACATTGAGCGCTCCTTCCCCGTATTCCTGAGGAAGGCCTTCCGCTCT | |
| GGGGAGATGGTCACCGTGGGCAAGAGCTCGGACGGCACTCCTGACCGCAGGTGGTGC | |
| TTCAGGGTGGATGAGGTGAACTGGTCTCACTGGAACCAGAACTTGGGCATCATCAACG | |
| AGGACCCGGGCAAGAATGAGACCTACCAGTATTATGGCTTCTCGCATACCGTGGGCCG | |
| CCTCCGCAGGGATCGCTGGTCCTCGGTGGTACCCCGCGTGGTGGAACTGAACAAGAAC | |
| TCGAACCCGGACGAGGTGGTGGTGCCTCTGGACAGCATGGGGAACCCCCGCTGCGATG | |
| GCCACCAGCAGGGTTACCCCCGCAAGTGGAGGACTGAGGACGCCCCGCTCTAG, | |

or a partial sequence thereof, a nucleic acid which is capable of hybridising with said sequence under stringent conditions, an allelic variant or a functional variant of said sequence or a variant of nucleic acid on the basis of the degenerative code. According to the invention, the sequence shown above comprises the human OTRPC4 cDNA sequence.

Also preferred is a nucleic acid which is characterised in that it has the sequence:

| | |
|---|---|
| ATGGCGGATTCCAGCGAAGGCCCCCGCGCGGGGCCCGGGGAGGTGGCTGAGCTCCCC | (SEQ ID NO:_) |
| GGGGATGAGAGTGGCACCCCAGGTGGGGAGGCTTTTCCTCTCTCGTCCCTGGCCAATC | |
| TGTTTGAGGGGGAGGATGGCTCCCTTTCGCCCTCACCGGCTGATGCCAGTCGCCCTGCT | |
| GGCCCAGGCGATGGGCGACCAAATCTGCGCATGAAGTTCCAGGGCGCCTTCCGCAAGG | |
| GGGTGCCCAACCCCATCGATCTGCTGGAGTCCACCCTATATGAGTCCTCGGTGGTGCCT | |
| GGGCCCAAGAAAGCACCCATGGACTCACTGTTTGACTACGGCACCTATCGTCACCACT | |
| CCAGTGACAACAAGAGGTGGAGGAAGAAGATCATAGAGAAGCAGCCGCAGAGCCCCA | |
| AAGCCCCTGCCCCTCAGCCGCCCCCCATCCTCAAAGTCTTCAACCGGCCTATCCTCTTT | |
| GACATCGTGTCCCGGGGCTCCACTGCTGACCTGGACGGGCTGCTCCCATTCTTGCTGAC | |
| CCACAAGAAACGCCTAACTGATGAGGAGTTTCGAGAGCCATCTACGGGGAAGACCTG | |
| CCTGCCCAAGGCCTTGCTGAACCTGAGCAATGGCCGCAACGACACCATCCCTGTGCTG | |
| CTGGACATCGCGGAGCGCACCGGCAACATGCGGGAGTTCATTAACTCGCCCTTCCGTG | |
| ACATCTACTATCGAGGTCAGACAGCCCTGCACATCGCCATTGAGCGTCGCTGCAAACA | |
| CTACGTGGAACTTCTCGTGGCCCAGGGAGCTGATGTCCACGCCCAGGCCCGTGGGCGC | |
| TTCTTCCAGCCCAAGGATGAGGGGGGCTACTTCTACTTTGGGGAGCTGCCCCTGTCGCT | |
| GGCTGCCTGCACCAACCAGCCCCACATTGTCAACTACCTGACGGAGAACCCCCACAAG | |
| AAGGCGGACATGCGGCGCCAGGACTCGCGAGGCAACACAGTGCTGCATGCGCTGGTG | |
| GCCATTGCTGACAACACCCGTGAGAACACCAAGTTTGTTACCAAGATGTACGACCTGC | |
| TGCTGCTCAAGTGTGCCCGCCTCTTCCCCGACAGCAACCTGGAGGCCGTGCTCAACAA | |
| CGACGGCCTCTCGCCCCTCATGATGGCTGCCAAGACGGGCAAGATTGGGATCTTTCAG | |
| CACATCATCCGGCGGGAGGTGACGGATGAGGACACACGGCACCTGTCCCGCAAGTTCA | |
| AGGACTGGGCCTATGGGCCAGTGTATTCCTCGCTTTATGACCTCTCCTCCCTGGACACG | |
| TGTGGGGAAGAGGCCTCCGTGCTGGAGATCCTGGTGTACAACAGCAAGATTGAGAACC | |
| GCCACGAGATGCTGGCTGTGGAGCCCATCAATGAACTGCTGCGGGACAAGTGGCGCA | |
| AGTTCGGGGCCGTCTCCTTCTACATCAACGTGGTCTCCTACCTGTGTGCCATGGTCATC | |
| TTCACTCTCACCGCCTACTACCAGCCGCTGGAGGGCACACCGCCGTACCCTTACCGCAC | |
| CACGGTGGACTACCTGCGGCTGGCTGGCGAGGTCATTACGCTCTTCACTGGGGTCCTGT | |
| TCTTCTTCACCAACATCAAAGACTTGTTCATGAAGAAATGCCCTGGAGTGAATTCTCTC | |
| TTCATTGATGGCTCCTTCCAGCTGCTCTACTTCATCTACTCTGTCCTGGTGATCGTCTCA | |
| GCAGCCCTCTACCTGGCAGGGATCGAGGCCTACCTGGCCGTGATGGTCTTTGCCCTGGT | |
| CCTGGGCTGGATGAATGCCCTTTACTTCACCCGTGGGCTGAAGCTGACGGGGACCTAT | |
| AGCATCATGATCCAGAAGATTCTCTTCAAGGACCTTTTCCGATTCCTGCTCGTCTACTT | |
| GCTCTTCATGATCGGCTACGCTTCAGCCCTGGTCTCCCTCCTGAACCCGTGTGCCAACA | |
| TGAAGGTGTGCAATGAGGACCAGACCAACTGCACAGTGCCCACTTACCCCTCGTGCCG | |
| TGACAGCGAGACCTTCAGCACCTTCCTCCTGGACCTGTTTAAGCTGACCATCGGCATGG | |
| GCGACCTGGAGATGCTGAGCAGCACCAAGTACCCCGTGGTCTTCATCATCCTGCTGGT | |
| GACCTACATCATCCTCACCTTTGTGCTGCTCCTCAACATGCTCATTGCCCTCATGGGCG | |
| AGACAGTGGGCCAGGTCTCCAAGGAGAGCAAGCACATCTGGAAGCTGCAGTGGGCCA | |
| CCACCATCCTGGACATTGAGCGCTCCTTCCCCGTATTCCTGAGGAAGGCCTTCCGCTCT | |
| GGGGAGATGGTCACCGTGGGCAAGAGCTCGGACGGCACTCCTGACCGGAGGTGGTGC | |
| TTCAGGGTGGATGAGGTGAACTGGTCTCACTGGAACCAGAACTTGGGCATCATCAACG | |
| AGGACCCGGGCAAGAATGAGACCTACCAGTATTATGGCTTCTCGCATACCGTGGGCCG | |
| CCTCCGCAGGGATCGCTGGTCCTCGGTGGTACCCCGCGTGGTGGAACTGAAGAAGAAC | |
| TCGAACCCGGACGAGGTGGTGGTGCCTCTGGACAGCATGGGGAACCCCCGCTGCGATG | |
| GCCACCAGCAGGGTTACCCCCGCAAGTGGAGGACTGAGGACGCCCCGCTCTAG. | |

The sequence shown above is, according to the invention, the human OTRPC4 cDNA sequence.

Also preferred is a polypeptide which is characterised in that it contains the amino acid sequence:

| | |
|---|---|
| MADSSEGPRAGPGEVAELPGDESGTPGGEAFPLSSLANLFEGEDGSLSPSPADASRPAGPG | (SEQ ID NO:_) |
| DGRPNLRMKFQGAFRKGVPNPJDLLESTLYESSVVPGPKKAPMDSLFDYGTYRHSSDNK | |
| RWRKKIIEKQPQSPKAPAPQPPPILKVFNRPILFDIVSRGSTADLDGLLPFLLTHKKRLTDEEF | |
| REPSTGKTCLPKALLNLSNGRNDTIPVLLDIAERTGNMRLEFINSPFRDIYYRGQTALHIAIER | |
| RCKHYVELLVAQGADVHAQARGRFFQPKDEGGYFYFGELPLSLAACTNQPHIVNYLTENP | |
| HKKADMRRQDSRGNTVLHALVAIADNTRENTKFVTKMYDLLLLKCARLFPDSNLEAVLN | |
| NDGLSPLMMAAKTGKIGIFQHIIRREVTDEDTRHLSRLKFKDWAYGPVYSSLYDLSSLDTCG | |
| EEASVLEILVYNSKIENRHEMLAVEPJNELLRDKWRKFGAVSFYINVVSYLCAMVIFTLTA | |
| YYQPLEGTPPYPYRTTVDYLRLAGEVITLFTGVLFFFTNIKDLFMKKCPGVNSLFIDGSFQL | |
| LYFIYSVLVIVSAALYLAGIEAYLAVMVFALVLGWMNALYFTRGLKLTGTYSIMIQKILFK | |
| DLFRFLLVYLLFMIGYASALVSLLNPCANMKVCNEDQTNCTVPTYPSCRDSETFSTFLLDL | |
| FKLTIGMGDLEMLSSTKYPVVFIILLVTYIILTFVLLLNMLIALMGETVGQVSKESKHIWKL | |
| QWATTILDIERSFPVFLRKAFRSGEMVTVGKSSDGTPDRRWCFRVDEVNWSHWNQNLGII | |
| NEDPGKNETYQYYGFSHTVGRLRRDRWSSVVPRVVELNKNSNPDEVVVPLDSMGNPRCD | |
| GHQQGYPRLKWRTEDAPL. | |

The sequence shown above is, according to the invention, the amino acid sequence of Human ORTPC4.

Also preferred is a nucleic acid which is characterised in that it contains the sequence

| | |
|---|---|
| GGCCACGCGTCGACTAGTACGGGGGGGGGGGGGGGGGTGGCRGSRGGAKCAGGACTC | (SEQ ID NO:_) |
| GGCCGGAGGGATCAGGAAGCGGCGGCGCTGCGCCCGCGTCCTGAGGCTGAGAAGTAC | |
| AAACAGATCTGGGTCCAGTATGGCAGATCCTGGTGATGGTCCCCGTGCAGCGCCTGGG | |
| GAGGTGGCTGAGCCCCCTGGAGATGAGAGTGGTACCTCTGGTGGGGAGGCCTTCCCCG | |
| TCTCTTCCCTGGCCAATCTGTTTGAGGGGGAGGAAGGCTCCTCTTCTCTTTCCCCGGTG | |
| GATGCTAGCCGCCCTGCTGGCCCTGGCGATGGACGTCCAAACCTGCGTATGAAGTTCC | |
| AGGGCGCTTTCCGCAAGGGGGTTCCCAACCCCATTGACCTGTTGGAGTCCACCCGGTA | |
| CGAGTCCTCAGTAGTGCCTGGGCCCAAGAAAGCGCCCATGGATTCCTTGTTCGACTAC | |
| GGCACTTACCGTCACCACCCCAGTGACAACAAGAGATGGAGGAGAAAGGTCGTGGAG | |
| AAGCAGCCACAGAGCCCCAAAGCTCCTGCACCCCAGCCACCCCCCATCCTCAAAGTCT | |
| TCAATCGGCCCATCCTCTTTGACATTGTGTCCCGGGGCTCCACTGCGGACCTAGATGGA | |
| CTGCTCTCCTTCTTGTTGACCCACAAGAAGCGCCTGACTGATGAGGAGTTCCGGGAGC | |
| CGTCCACGGGGAAGACCTGCCTGCCCAAGGCGCTGCTGAACCTAAGCAACGGGCGCA | |
| ACGACACCATCCCGGTGTTGCTGGACATTGCGGAGCGCACCGGCAACATGCGTGAATT | |
| CATCAACTCGCCCTTCAGAGACATCTACTACCGAGGCCAGACATCCCTGCACATTGCC | |
| ATCGAACGGCGCTGCAAGCACTACGTGGAGCTGCTGGTGGCCCAGGGAGCCGACGTG | |
| CACGCCCAGGCCCGCGGCCGCTTCTTCCAGCCCAAGGATGAGGGAGGCTACTTCTACT | |
| TTGGGGAGCTGCCCTTGTCCCTGGCAGCCTGCACCAACCAGCCGCACATCGTCAACTA | |
| CCTGACAGAGAACCCTCACAAGAAAGCTGACATGAGGCGACAGGACTCGAGGGGGAA | |
| CACGGTGCTGCACGCGCTGGTGGCCATCGCCGACAACACCCGAGAGAACACCAAGTTT | |
| GTGACCAAGATGTACGACCTGCTGCTTCTCAAGTGTTCACGCCTCTTCCCCGACAGCAA | |
| CCTGGAGACAGTTCTCAACAATGATGGCCTTTCGCCTCTCATGATGGCTGCCAAGACA | |
| GGCAAGATCGGGGTCTTTCAGCACATCATCCGACGTGAGGTGACAGATGAGGACACCC | |
| GGCATCTGTCTCGCAAGTTCAAGGACTGGGCCTATGGGCCTGTGTATTCTTCTCTCTAC | |
| GACCTCTCCTCCCTGGACACATGCGGGGAGGAGGTGTCCGTGCTGGAGATCCTGGTGT | |
| ACAACAGCAAGATCGAGAACCGCCATGAGATGCTGGCTGTAGAGCCCATTAACGAAC | |
| TGTTGAGAGACAAGTGGCGTAAGTTTGGGGCTGTGTCCTTCTACATCAACGTGGTCTCC | |
| TATCTGTGTGCCATGGTCATCTTCACCCTCACCGCCTACTATCAGCCACTGGAGGGCAG | |
| GCCACCCTACGCTTACCGGACCACAGTGGACTACCTGAGGCTGGCTGGCGAGGTCATC | |
| ACGCTCTTCACAGGAGTCCTGTTCTTCTTTACCAGTATCAAGACTTGTTCACGAAGAA | |
| ATGCCCTGGAGTGAATTCTCTCTTCGTCGATGGCTCCTTCCAGTTACTCTACTTCATCTA | |
| CTCTGTGCTGGTGGTTGTCTCTGCGGCGCTCTACCTGGCTGGGATCGAGGCCTACCTGG | |
| CTGTGATGGTCTTTGCCCTGGTCCTGGGCTGGATGAATGCGCTGTACTTCACGCGCGGG | |
| TTGAAGCTGACGGGGACCTACAGCATCATGATTCAGAAGATCCTCTTCAAAGACCTCT | |
| TCCGCTTCCTGCTTGTGTACCTGCTCTTCATGATCGGCTATGCCTCAGCCCTGGTCACCC | |
| TCCTGAATCCGTGCACCAACATGAAGGTCTGTGACGAGGACCAGAGCAACTGCACGGT | |
| GCCCACGTATCCTGCGTGCCGCGACAGCGAGACCTTCAGCGCCTTCCTCCTGGACCTCT | |
| TCAAGCTCACCATCGGCATGGGAGACCTGGAGATGCTGAGCAGCGCCAAGTACCCCGT | |
| GGTCTTCATCCTCCTGCTGGTCACCTACATCATCCTCACCTTCGTGCTCCTGTTGAACAT | |
| GCTTATCGCCCTCATGGGTGAGACCGTGGGCCAGGTGTCCAAGGAGAGCAAGCACATC | |
| TGGAAGTTGCAGTGGGCCACCACCATCCTGGACATCGAGCGTTCCTTCCCTGTGTTCCT | |
| GAGGAAGGCCTTCCGCTCCGGAGAGATGGTGACTGTGGGCAAGAGCTCAGATGGCAC | |
| TCCGGACCGCAGGTGGTGCTTCAGGGTGGACGAGGTGAACTGGTCTCACTGGAACCAG | |
| AACTTGGGCATCATTAACGAGGACCCTGGCAAGAGTGAAATCTACCAGTACTATGGCT | |
| TCTCCCACACCGTGGGGCGCCTTCGTAGGGATCGTTGGTCCTCGGTGGTGCCCCGCGTA | |
| GTGGAGCTGAACAAGAACTCAAGCGCAGATGAAGTGGTGGTACCCCTGGATAACCTA | |
| GGGAACCCCAACTGTGACGGCCACCAGCAGGGCTACGCTCCCAAGTGGAGGACGGAC | |
| GATGCCCCACTGTAGGGGCCGTGCCAGAGCTCGCACAGATAGTCCAGGCTTGGCCTTC | |
| GCTCCCACCTACATTTAGGCATTTGTCCGGTGTCTTCCCACACCCGCATGGGACCTTGG | |
| AGGTGAGGGCCTCTGTGGCGACTCTGTGGAGGCCCCAGGACCCTCTGGTCCCCGCCAA | |
| GACTTTTGCCTTCAGCTCTACTCCCCACATGGGGGGGCGGGGCTCCTGGCTACCTGTCT | |
| CGCTCGCTCCCATGGAGTCACCTAAGCCAGCACAAGGCCCCTCTCCTCGAAAGGCTCA | |
| GGCCCCATCCCTCTTGTGTATTATTTATTGCTCTCCTCAGGAAAATGGGGTGGCAGGAG | |
| TCCACCCGCGGCTGGAACCTGGCCAGGGCTGAAGCTCATGCAGGGACGCTGCAGCTCC | |
| GACCTGCCACAGATCTGACCTGCTGCAGCCCTGGCTAGTGTGGGTCTTCTGTACTTTGA | |
| AGAGATCGGGGCCGCTGGTGCTCAATAAATGTTTATTCTCGGTGGAAAAAAAAAAAAA | |
| AAAAAAAAAAAAAAAAAAAAAAAAAAAAAAAAAAAAAAAAAAAAAAAAAAAAAAA | |
| AAAAAAAAA. | |

or a partial sequence thereof, a nucleic acid which is capable of hybridising with said sequence under stringent conditions, an allelic variant or a functional variant of said sequence or a variant of the nucleic acid on the basis of the degenerative code, wherein R may be an A or G, M may be an A or C, S may be a C or G, Y may be a C or T, K may be a G or T and W may be an A or T. According to the invention, the sequence shown above comprises the murine OTRPC4 DNA sequence with 5′ and 3′-untranslated sequences.

Also preferred is a nucleic acid which is characterised in that it has the sequence

| | |
|---|---|
| GGCCACGCGTCGACTAGTACGGGGGGGGGGGGGGGGGTGGCRGSRGGAKCAGGACTC | (SEQ ID NO:_) |
| GGCCGGAGGGATCAGGAAGCGGCGGCGCTGCGCCCGCGTCCTGAGGCTGAGAAGTAC | |
| AAACAGATCTGGGTCCAGTATGGCAGATCCTGGTGATGGTCCCCGTGCAGCGCCTGGG | |
| GAGGTGGCTGAGCCCCCTGGAGATGAGAGTGGTACCTCTGGTGGGGAGGCCTTCCCCC | |
| TCTCTTCCCTGGCCAATCTGTTTGAGGGGGAGGAAGGCTCCTCTTCTCTTTCCCCGGTG | |
| GATGCTAGCCGCCCTGCTGGCCCTGGCGATGGACGTCCAAACCTGCGTATGAAGTTCC | |
| AGGGCGCTTTCCGCAAGGGGGTTCCCAACCCCATTGACGTGTTGGAGTCCACCCGGTA | |
| CGAGTCCTCAGTAGTGCCTGGGCCCAAGAAAGCGCCCATGGATTCCTTGTTCGACTAC | |
| GGCACTTACCGTCACCACCCCAGTGACAACAAGAGATGGAGGAGAAAGGTCGTGGAG | |
| AAGCAGCCACAGAGCCCCAAAGCTCCTGCACCCCAGCCACCCCCCATCCTCAAAGTCT | |
| TCAATCGGCCCATCCTCTTTGACATTGTGTCCCGGGGCTCCACTGCGGACCTAGATGGA | |
| CTGCTCTCCTTCTTGTTGACCCACAAGAAGCGCCTGACTGATGAGGAGTTCGGGGAGC | |
| CGTCCACGGGGAAGACCTGCCTGCCCAAGGCGCTGCTGAACCTAAGCAACGGGCGCA | |
| ACGACACCATCCCGGTGTTGCTGGACATTGCGGAGCGCACCGGCAACATGCGTGAATT | |
| CATCAACTCGCCCTTCAGAGACATCTACTACCGAGGCCAGACATCCCTGCACATTGCC | |
| ATCGAACGGCGCTGCAAGCACTACGTGGAGCTGCTGGTGGCCCAGGGAGCCGACGTG | |
| CACGCCCAGGCCCGCGGCCGCTTCTTCCAGCCCAAGGATGAGGGAGGCTACTTCTACT | |
| TTGGGGAGCTGCCCTTGTCCCTGGCAGCCTGCACCAACCAGCCGCACATCGTCAACTA | |
| CCTGACAGAGAACCCTCACAAGAAAGCTGACATGAGGCGACAGGACTCGAGGGGGAA | |
| CACGGTGCTGCACGCGCTGGTGGCCATCGCCGACAACACCCGAGAGAACACCAAGTTT | |
| GTCACCAAGATGTACGACCTGCTGCTTCTCAAGTGTTCACGCCTCTTCCCCGACAGCAA | |
| CCTGGAGACAGTTCTCAACAATGATGGCCTTTCGCCTCTCATGATGGCTGCCAAGACA | |
| GGCAAGATCGGGGTCTTTCAGCACATCATCCGACGTGAGGTGACAGATGAGGACACCC | |
| GGCATCTGTCTCGCAAGTTCAAGGACTGGGCCTATGGGCCTGTGTATTCTTCTCTCTAC | |
| GACCTCTCCTCCCTGGACACATGCGGGGAGGAGGTGTCCGTGCTGGAGATCCTGGTGT | |
| ACAACAGCAAGATCGAGAACCGCCATGAGATGCTGGCTGTAGAGCCCATTAACGAAC | |
| TGTTGAGAGACAAGTGGCGTAAGTTTGGGGCTGTGTCCTTCTACATCAACGTGGTCTCC | |
| TATCTGTGTGCCATGGTCATCTTCACCCTCACCGCCTACTATCAGCCACTGGAGGGCAC | |
| GCCACCCTACCCTTACCGGACCACAGTGGACTACCTGAGGCTGGCTGGCGAGGTCATC | |
| AGGCTCTTCAGAGGAGTCCTGTTCTTCTTTACCAGTATCAAAGACTTGTTCACGAAGAA | |
| ATGCCCTGGAGTGAATTCTCTCTTCGTCGATGGCTCCTTCCAGTTACTCTACTTCATCTA | |
| CTCTGTGCTGGTGGTTGTCTCTGCGGCGCTCTACCTGGCTGGGATCGAGGCCTACCTGG | |
| CTGTGATGGTCTTTGCCCTGGTCCTGGGCTGGATGAATGCGCTGTACTTCACGCGCGGG | |
| TTGAAGCTGACGGGGACCTACAGCATCATGATTCAGAAGATCCTCTTCAAAGACCTCT | |
| TCCGCTTCCTGCTTGTGTACCTGCTCTTCATGATCGGCTATGCCTCAGCCCTGGTCACCC | |
| TCCTGAATCCGTGCACCAACATGAAGGTCTGTGACGAGGACCAGAGCAACTGCACGGT | |
| GCCCACGTATCCTGCGTGCCGCGACAGCGAGACCTTCAGCGCCTTCCTCCTGGACCTCT | |
| TCAAGCTCACCATCGGCATGGGAGACCTGGAGATGCTGAGCAGCGCCAAGTACCCCGT | |
| GGTCTTCATCCTCCTGCTGGTCACCTACATCATCCTCACCTTCGTGCTCCTGTTGAACAT | |
| GCTTATCGCCCTCATGGGTGAGACCGTGGGCCAGGTGTCCAAGGAGAGCAAGCACATC | |
| TGGAAGTTGCAGTGGGCCACCACCATCCTGGACATCGAGCGTTCCTTCCCTGTGTTCCT | |
| GAGGAAGGCCTTCCGCTCCGGAGAGATGGTGACTGTGGGCAAGAGCTCAGATGGCAC | |
| TCCGGACCGCAGGTGGTGCTTCAGGGTGGACGAGGTGAACTGGTCTCACTGGAACCAG | |
| AACTTGGGCATCATTAACGAGGACCCTGGCAAGAGTGAAATCTACCAGTACTATGGCT | |
| TCTCCCACACCGTGGGGCGCCTTCGTAGGGATCGTTGGTCCTCGGTGGTGCCCCGCGTA | |
| GTGGAGCTGAACAAGAACTCAAGCGCAGATGAAGTGGTGGTACCCCTGGATAACCTA | |
| GGGAACCCCAACTGTGACGGCCACCAGCAGGGCTACGCTCCCAAGTGGAGGACGGAC | |
| GATGCCCCACTGTAGGGGCCGTGCCAGAGCTCGCACAGATAGTCCAGGCTTGGCCTTC | |
| GCTCCCACCTACATTTAGGCATTTGTCCGGTGTCTTCCCACACCCGCATGGGACCTTGG | |
| AGGTGAGGGCCTCTGTGGCGACTCTGTGGAGGCCCCAGGACCCTCTGGTCCCCGCCAA | |
| GACTTTTGCCTTCAGCTCTACTCCCCACATGGGGGGGCGGGGCTCCTGGCTACCTGTCT | |
| CGCTCGCTCCCATGGAGTCACCTAAGCCAGCACAAGGCCCCTCTCCTCGAAAGGCTCA | |
| GGCCCCATCCCTCTTGTGTATTATTTATTGCTCTCCTCAGGAAAATGGGGTGGCAGGAG | |
| TCCACCCGCGGCTGGAACCTGGCCAGGGCTGAAGCTCATGCAGGGACGCTGCAGCTCC | |
| GACCTGCCACAGATCTGACCTGCTGCAGCCCTGGCTAGTGTGGGTCTTCTGTACTTTGA | |
| AGAGATCGGGGCCGCTGGTGCTCAATAAATGTTTATTCTCGGTGGAAAAAAAAAAAAA | |
| AAAAAAAAAAAAAAAAAAAAAAAAAAAAAAAAAAAAAAAAAAAAAAAAAAAAAAA | |
| AAAAAAAAA, | |

wherein R may be an A or G, M may be an A or C, S may be a C or G, Y may be a C or T, K may be a G or T and W may be an A or T. The sequence shown above is, according to the invention, the murine OTRPC4 DNA sequence with 5′ and 3′-untranslated sequences.

Also preferred is a nucleic acid which is characterised in that it contains the sequence

| | |
|---|---|
| ATGGCAGATCCTGGTGATGGTCCCCGTGCAGCGCCTGGGGAGGTGGCTGAGCCCCCTG | (SEQ ID NO_:) |
| GAGATGAGAGTGGTACCTCTGGTGGGGAGGCCTTCCCCCTCTCTTCCCTGGCCAATCTG | |
| TTTGAGGGGGAGGAAGGCTCCTCTTCTCTTTCCCCGGTGGATGCTAGCCGCCCTGCTGG | |
| CCCTGGCGATGGACGTCCAAACCTGCGTATGAAGTTCCAGGGCGCTTTCCGCAAGGGG | |
| GTTCCCAACCCCATTGACCTGTTGGAGTCCACCCGGTACGAGTCCTCAGTAGTGCCTGG | |
| GCCCAAGAAAGCGCCCATGGATTCCTTGTTCGACTACGGCACTTACCGTCACCACCCC | |
| AGTGACAACAAGAGATGGAGGAGAAAGGTCGTGGAGAAGCAGCCACAGAGCCCCAA | |
| AGCTCCTGCACCCCAGCCACCCCCCATCCTCAAAGTCTTCAATCGGCCCATCCTCTTTG | |
| ACATTGTGTCCCGGGGCTCCACTGCGGACCTAGATGGACTGCTCTCCTTCTTGTTGACC | |
| CACAAGAAGCGCCTGACTGATGAGGAGTTCCGGGAGCCGTCCACGGGGAAGACCTGC | |
| CTGCCCAAGGGGCTGCTGAACCTAAGCAACGGGCGCAACGACACCATCCCGGTGTTGC | |
| TGGACATTGCGGAGCGCACCGGCAACATGCGTGAATTCATCAACTCGCCCTTCAGAGA | |
| CATCTACTACCGAGGCCAGACATCCCTGCACATTGCCATCGAACGGCGCTGCAAGCAC | |
| TACGTGGAGCTGCTGGTGGCCCAGGGAGCCGACGTGCACGCCCAGGCCCGCGGCCGCT | |
| TCTTCCAGCCCAAGGATGAGGGAGGCTACTTCTACTTTGGGGAGCTGCCCTTGTCCCTG | |
| GCAGCCTGCACCAACCAGCCGCACATCGTCAACTACCTGACAGAGAACCCTCACAAGA | |
| AAGCTGACATGAGGCGACAGGACTCGAGGGGGAACACGGTGCTGCACGCGCTGGTGG | |
| CCATCGCCGACAACACCCGAGAGAACACCAAGTTTGTCACCAAGATGTACGACCTGCT | |
| GCTTCTCAAGTGTTCACGCCTCTTCCCCGACAGCAACCTGGAGACAGTTCTCAACAATG | |
| ATGGCCTTTCGCCTCTCATGATGGCTGCCAAGACAGGCAAGATCGGGGTCTTTCAGCA | |
| CATCATCCGACGTGAGGTGACAGATGAGGACACCCGGCATCTGTCTCGCAAGTTCAAG | |
| GACTGGGCCTATGGGCCTGTGTATTCTTCTCTCTACGACCTCTCCTCCCTGGACACATG | |
| CGGGGAGGAGGTGTCCGTGCTGGAGATCCTGGTGTACAACAGCAAGATCGAGAACCG | |
| CCATGAGATGCTGGCTGTAGAGCCCATTAACGAACTGTTGAGAGACAAGTGGCGTAAG | |
| TTTGGGGCTGTGTCCTTCTACATCAACGTGGTCTCCTATCTGTGTGCCATGGTCATCTTC | |
| ACCCTCACCGCCTACTATCAGCCACTGGAGGGCACGCCACCCTACCCTTACCGGACCA | |
| CAGTGGACTACCTGAGGCTGGCTGGCGAGGTCATCACGCTCTTCACAGGAGTCCTGTT | |
| CTTCTTTACCAGTATCAAAGACTTGTTCACGAAGAAATGCCCTGGAGTGAATTCTCTCT | |
| TCGTCGATGGCTCCTTCCAGTTACTCTACTTCATCTACTCTGTGCTGGTGGTTGTCTCTG | |
| CGGCGCTCTACCTGGCTGGGATCGAGGCCTACCTGGCTGTGATGGTCTTTGCCCTGGTC | |
| CTGGGCTGGATGAATGCGCTGTACTTCACGCGCGGGTTGAAGCTGACGGGGACCTACA | |
| GCATCATGATTCAGAAGATCCTCTTCAAAGACCTCTTCCGCTTCCTGCTTGTGTACCTG | |
| CTCTTCATGATCGGCTATGCCTCAGCCCTGGTCACCCTCCTGAATCCGTGCACCAACAT | |
| GAAGGTCTGTGACGAGGACCAGAGCAACTGCACGGTGCCCACGTATCCTGCGTGCCGC | |
| GACAGCGAGACCTTCAGCGCCTTCCTCCTGGACCTCTTCAAGCTCACCATCGGCATGG | |
| GAGACCTGGAGATGCTGAGCAGCGCCAAGTACCCCGTGGTCTTCATCCTCCTGCTGGT | |
| CACCTACATCATCCTCACCTTCGTGCTCCTGTTGAACATGCTTATCGCCCTCATGGGTG | |
| AGACCGTGGGCCAGGTGTCCAAGGAGAGCAAGCACATCTGGAAGTTGCAGTGGGCCA | |
| CCACCATCCTGGACATCGAGCGTTCCTTCCCTGTGTTCCTGAGGAAGGCCTTCCGCTCC | |
| GGAGAGATGGTGACTGTGGGCAAGAGCTCAGATGGCACTCCGGACCGCAGGTGGTGC | |
| TTCAGGGTGGACGAGGTGAACTGGTCTCACTGGAACCAGAACTTGGGCATCATTAACG | |
| AGGACCCTGGCAAGAGTGAAATCTACCAGTACTATGGCTTCTCCCACACCGTGGGGCG | |
| CCTTCGTAGGGATCGTTGGTCCTCGGTGGTGCCCCGCGTAGTGGAGCTGAACAAGAAC | |
| TCAAGCGCAGATGAAGTGGTGGTACCCCTGGATAACCTAGGGAACCCCAACTGTGACG | |
| GCCACCAGCAGGGCTACGCTCCCAAGTGGAGGACGGACGATGCCCCACTGTAG, | |

or a partial sequence thereof, a nucleic acid which is capable of hybridising with said sequence under stringent conditions, an allelic variant or a functional variant of said sequence or a variant of the nucleic acid on the basis of the degenerative code. According to the invention, the sequence shown above comprises the murine OTRPC4 cDNA sequence.

Also preferred is a nucleic acid which is characterised in that it has the sequence

| | |
|---|---|
| ATGGCAGATCCTGGTGATGGTCCCCGTGCAGCGCCTGGGGAGGTGGCTGAGCCCCCTG | (SEQ ID NO:_) |
| GAGATGAGAGTGGTACCTCTGGTGGGGAGGCCTTCCCCCTCTCTTCCCTGGCCAATCTG | |
| TTTGAGGGGGAGGAAGGCTCCTCTTCTCTTTCCCCGGTGGATGCTAGCCGCCCTGCTGG | |
| CCCTGGCGATGGACGTCCAAACCTGCGTATGAAGTTCCAGGGCGCTTTCCGCAAGGGG | |
| GTTCCCAACCCCATTGACCTGTTGGAGTCCACCCGGTACGAGTCCTCAGTAGTGCCTGG | |
| GCCCAAGAAAGCGCCCATGGATTCCTTGTTCGACTACGGCACTTACCGTCACCACCCC | |
| AGTGACAACAAGAGATGGAGGAGAAAGGTCGTGGAGAAGCAGCCACAGAGCCCCAA | |
| AGCTCCTGCACCCCAGCCACCCCCCATCCTCAAAGTCTTCAATCGGCCCATCCTCTTTG | |
| ACATTGTGTCCCGGGGCTCCACTGCGGACCTAGATGGACTGCTCTCCTTCTTGTTGACC | |
| CACAAGAAGCGCCTGACTGATGAGGAGTTCCGGGAGCCGTCCACGGGGAAGACCTGC | |
| CTGCCCAAGGCGCTGCTGAACCTAAGCAACGGGCGCAACGACACCATCCCGGTGTTGC | |
| TGGACATTGCGGAGCGCACCGGCAACATGCGTGAATTCATCAACTCGCCCTTCAGAGA | |
| CATCTACTACCGAGGCCAGACATCCCTGCACATTGCCATCGAACGGCGCTGCAAGCAC | |
| TACGTGGAGCTGCTGGTGGCCCAGGGAGCCGACGTGCACGCCCAGGCCCGCGGCCGCT | |
| TCTTCCAGCCCAAGGATGAGGGAGGCTACTTCTACTTTGGGGAGCTGCCCTTGTCCCTG | |
| GCAGCCTGCACCAACCAGCCGCACATCGTCAACTACCTGACAGAGAACCCTCACAAGA | |
| AAGCTGACATGAGGCGACAGGACTCGAGGGGGAACACGGTGCTGCACGCGCTGGTGG | |
| CCATCGCCGACAACACCCGAGAGAACACCAAGTTTGTCACCAAGATGTACGACCTGCT | |
| GCTTCTCAAGTGTTCACGCCTCTTCCCCGACAGCAACCTGGAGACAGTTCTCAACAATG | |
| ATGGCCTTTCGCCTCTCATGATGGCTGCCAAGACAGGCAAGATCGGGGTCTTTCAGCA | |
| CATCATCCGACGTGAGGTGACAGATGAGGACACCCGGCATCTGTCTCGCAAGTTCAAG | |
| GACTGGGCCTATGGGCCTGTGTATTCTTCTCTCTACGACCTCTCCTCCCTGGACACATG | |
| CGGGGAGGAGGTGTCCGTGCTGGAGATCCTGGTGTACAACAGCAAGATCGAGAACCG | |
| CCATGAGATGCTGGCTGTAGAGCCCATTAACGAACTGTTGAGAGACAAGTGGCGTAAG | |
| TTTGGGGCTGTGTCCTTCTACATCAACGTGGTCTCCTATCTGTGTGCCATGGTCATCTTC | |
| ACCCTCACCGCCTACTATCAGCCACTGGAGGGCACGCCACCCTACCCTTACCGGACCA | |
| CAGTGGACTACCTGAGGCTGGCTGGCGAGGTCATCACGCTCTTCACAGGAGTCCTGTT | |
| CTTCTTTACCAGTATCAAAGACTTGTTCACGAAGAAATGCCCTGGAGTGAATTCTCTCT | |
| TCGTCGATGGCTCCTTCCAGTTACTCTACTTCATCTACTCTGTGCTGGTGGTTGTCTCTG | |
| CGGCGCTCTACCTGGCTGGGATCGAGGCCTACCTGGCTGTGATGGTCTTTGCCCTGGTC | |
| CTGGGCTGGATGAATGCGCTGTACTTCACGCGCGGGTTGAAGCTGACGGGGACCTACA | |
| GCATCATGATTCAGAAGATCCTCTTCAAAGACCTGTTCCGCTTCCTGCTTGTGTACCTG | |
| CTCTTCATGATCGGCTATGCCTCAGCCCTGGTCACCCTCCTGAATCCGTGCACCAACAT | |
| GAAGGTCTGTGACGAGGACCAGAGCAACTGCACGGTGCCCACGTATCCTGCGTGCCGC | |
| GACAGCGAGACCTTCAGCGCCTTCCTCCTGGACCTCTTCAAGCTCACCATCGGCATGG | |
| GAGACCTGGAGATGCTGAGCAGCGCCAAGTACCCCGTGGTCTTCATCCTCCTGCTGGT | |
| CACCTACATCATCCTCACCTTCGTGCTCCTGTTGAACATGCTTATCGCCCTCATGGGTG | |
| AGACCGTGGGCCAGGTGTCCAAGGAGAGCAAGCACATCTGGAAGTTGCAGTGGGCCA | |
| CCACCATCCTGGACATCGAGCGTTCCTTCCCTGTGTTCCTGAGGAAGGCCTTCCGCTCC | |
| GGAGAGATGGTGACTGTGGGCAAGAGCTCAGATGGCACTCCGGACCGCAGGTGGTGC | |
| TTCAGGGTGGACGAGGTGAACTGGTCTCACTGGAACCAGAACTTGGGCATCATTAACG | |
| AGGACCCTGGCAAGAGTGAAATCTACCAGTACTATGGCTTCTCCCACACCGTGGGGCG | |
| CCTTCGTAGGGATCGTTGGTCCTCGGTGGTGCCCCGCGTAGTGGAGCTGAACAAGAAC | |
| TCAAGCGCAGATGAAGTGGTGGTACCCCTGGATAACCTAGGGAACCCCAACTGTGACG | |
| GCCACCAGGAGGGCTACGCTCCCAAGTGGAGGACGGACGATGCCCCACTGTAG. | |

The sequence shown above is, according to the invention, the murine OTRPC4 cDNA sequence.

In another preferred embodiment, a recombinant vector is characterised in that it contains a nucleic acid according to the invention as described above. Examples of vectors according to the invention include viral vectors such as Vaccinia, Semliki-Forest virus and Adenovirus. Vectors for use in COS cells have the Simian virus (SV) 40 origin of replication and make it possible to achieve high copy numbers of plasmids. Vectors for use in insect cells are E. coli transfer vectors, for example, and contain the DNA coding for polyhedrin, for example, as their promoter.

In another preferred embodiment, a recombinant vector according to the invention is characterised in that it is an expression vector.

Yet another preferred embodiment of the invention is a host, characterised in that it contains a vector according to the invention. A host according to the invention expresses an OTRPC4 polypeptide according to the invention, e.g. on the cell surface, for example integrated in the plasma membrane. The hosts according to the invention may be transiently or stably transfected with one of said vectors. A host of this kind is described by way of example in Example 1, FIG. 4 of the invention.

Yet another preferred embodiment of the invention is a host according to the invention which is a eukaryotic host cell. Eukaryotic host cells according to the invention include fungi such as Pichia pastoris, Saccharomyces cerevisiae, Schizosaccharomyces and Trichoderma. Another preferred host according to the invention is an insect cell (e.g. from Spodoptera frugiperda Sf-9, with a Baculovirus expression system). Cells according to the invention also include oocytes, e.g. from frogs or toads. Hosts according to the invention may also be plant cells, e.g. from Nicotiana tabacum. The OTRPC4 polypeptides according to the invention are expressed particularly well in mammalian cells and cell lines. Consequently, a preferred host according to the invention is a mammalian cell. Examples of mammalian cells according to the invention are HEK293-, HeLa-, COS-, BHK- and CHO-cells.

Most preferably, therefore, a host according to the invention is an Sf9-, HEK293- or HeLa-cell.

Preferably, a host according to the invention is a bacteriophage. Baculovirus may be mentioned by way of example.

Another host according to the invention is a prokaryotic host cell. Examples of prokaryotic host cells according to the invention include Escherichia coli, Bacillus subtilis, Streptomyces and also Proteus mirabilis.

Another important aspect of the present invention relates to a polypeptide which is coded by a nucleic acid according to the invention or a fragment, a functional variant, an allelic variant, a subunit, a variant on the basis of the degenerative nucleic acid code or a glycosylation variant thereof. Within the scope of this invention, the term OTRPC4 polypeptide or fragment thereof may denote one or more of the polypeptides described here, i.e. a polypeptide selected from among fragments, allelic variants, functional subunits, variants based on the degenerative nucleic acid code, a chemical derivative thereof, a fusion protein with said polypeptide or a glycosylation variant of OTRPC4. OTRPC4 polypeptides according to the invention are preferably eukaryotic polypeptides, most preferably human or murine polypeptides but also those derived from rats, hamsters, goats, cattle, pigs, sheep, dogs, cats and monkeys and from other eukaryotes known in the art. OTRPC4 within the scope of this invention is a new cation channel which has the advantageous property, compared with the cation channels known from the prior art, that it is regulated by changes in the osomolarity of the extracellular medium. Thus, it represents a completely new generation of cation channels compared with those known from the prior art, which act as osmosensors and are responsible for regulating cell volume, for example. The channel activity is stimulated by lowering the osmolarity and inhibited by increasing it. For example, the channel is constitutively active at a physiological osmolarity of about 300 mosmol/l. The channel is nonselective in its ion permeability, i.e. it is permeable to all cations (Na⁺, K⁺, Ca²⁺) and exhibits a certain preference for Ca²+(P_{Ca}/P_{Na}: about 6), for example.

Another important aspect of the present invention relates to a polypeptide according to the invention which is a fragment of the nonselective cation channel OTRPC4. This denotes a part of the polypeptide according to the invention.

Yet another important aspect of the present invention relates to a polypeptide according to the invention which is a functional variant of the nonselective cation channel OTRPC4.

This refers to polypeptides which are substantially similar to OTRPC4 and have the same biological activity as OTRPC4 or have an inhibitory activity for OTRPC4. A variant of OTRPC4 can differ from OTRPC4 by the substitution, deletion or addition of one or more amino acids, preferably 1 to 10 amino acids. For example, the term functional variants refers to other members of the OTRPC4 family which also have the advantageous property of regulating the channel activity by osmolarity as described above.

Yet another important aspect of the present invention relates to a polypeptide according to the invention which is an allelic variant of the non-selective cation channel OTRPC4.

Yet another important aspect of the present invention relates to a polypeptide according to the invention which is a subunit of the non-selective cation channel OTRPC4. Ion channels are often made up of subunits, e.g. the AMPA receptor. Accordingly, the invention also includes subunits of the OTRPC4 cation channel. Yet another important aspect of the present invention relates to a polypeptide according to the invention which is a variant of the non-selective cation channel OTRPC4 based on the degenerative nucleic acid code.

Yet another important aspect of the present invention relates to a polypeptide according to the invention which is a chemical derivative of the non-selective cation channel OTRPC4. This denotes molecules which are produced from the OTRPC4 polypeptides according to the invention by chemical reactions such as iodinisation, acetylation, binding to an effector molecule or radioisotope or to a toxin.

Yet another important aspect of the present invention relates to a polypeptide according to the invention which is a fusion protein made up of the non-selective cation channel OTRPC4 and another protein. A fusion protein of this kind may, for example, be prepared by recombinant expression of the OTRPC4 nucleic acid according to the invention which is fused to another nucleic acid according to the invention which is fused to another nucleic acid which contains all the coding information “in frame”. This may be, for example, a marker protein or a reporter protein such as green fluorescent protein (GFP) or LacZ.

Other fusion partners are known to those skilled in the art.

Yet another important aspect of the present invention relates to a polypeptide according to the invention which is a glycosylation variant of the non-selective cation channel OTRPC4.

The invention includes processes for preparing polypeptides according to the invention, characterised in that a host according to the invention is cultivated and said polypeptide is expressed. The said hosts may, for example, be stably or transiently transfected with a vector or an expression vector which contains a nucleic acid coding for an OTRPC4 polypeptide or fragment. For example, the OTRPC4 polypeptide or fragment according to the invention is expressed on the cell surface of the host. However, said polypeptide may also be secreted into the medium. The OTRPC4 polypeptides or fragments according to the invention may be prepared in a process according to the invention in fungi such as Pichia pastoris, Saccharomyces cerevisiae, Schizosaccharomyces or Trichoderma, for example, with vectors which lead to surface expression.

The process according to the invention for preparing OTRPC4 polypeptides or fragments may also be carried out with insect cells, e.g. as a transient or stable expression system or Baculovirus expression system. For example, Sf-9 insect cells are infected with Autographa californica nuclear polyhedrosis virus (AcNPV) or related viruses, for example. The E. coli transfer vectors described above contain as promoter the DNA coding for polyhedrin, for example, behind which the DNA coding for the OTRPC4 polypeptide or fragment according to the invention is cloned. After identification of a—correct transfer vector clone in E. coli, this clone together with incomplete Baculovirus DNA is transfected into an insect cell and recombined with the Baculovirus DNA to form viable Baculoviruses. Using strong insect cell promoters, large quantities of the OTRPC4 polypeptide or fragment according to the invention are formed in a process according to the invention. Insect cell expression systems for the expression of OTRPC4 polypeptide or fragment are commercially available.

A mammalian expression system, e.g. in a host according to the invention, e.g. the HEK293 cell or the Hela cell, which contains, for example, a nucleic acid according to the invention coding for OTRPC4 or a fragment thereof, in an expression vector, may be used for the expression of the OTRPC4 cation channel, said host being cultivated under conditions known in the art and the OTRPC4 polypeptide or fragment being expressed on the cell surface, for example. One advantage of mammalian expression systems is that they enable very good glycosylation and folding conditions. Mammalian cells can be used with transient expression systems, stable expression systems and with viral expression systems such as Vaccinia, Semliki-Forest virus and Adenovirus, which are commercially obtainable. Transgenic animals such as cows, goats and mice are also suitable for a process according to the invention. Transgenic plants such as Nicotiana tabacum (tobacco) may also be used in a process according to the invention. These are particularly suitable for the preparation of OTRPC4 polypeptide or fragment according to the invention. After genomic integration of the nucleic acid according to the invention which codes for an OTRPC4 polypeptide or fragment according to the invention fused to a signal sequence, the surface expression of the OTRPC4 polypeptide or fragment or secretion into the interstitial space can be achieved.

Preparation with prokaryotic expression systems such as Escherichia coli, Bacillus subtilis, Streptomyces or Proteus mirabilis is preferably suitable for OTRPC4 fragments according to the invention but may also be used for the entire OTRPC4 polypeptide. The OTRPC4 polypeptides according to the invention are either prepared preferably on the surface, e.g. integrated in the outer coat, i.e. one of the two bacterial cell membranes or the peptidoglycan layer of the outer coat in the case of gram-negative bacteria or into the cell membrane in the case of gram-positive bacteria, or they are produced intracellularly, e.g. in inclusion bodies or by periplasmatic secretion in gram-negative bacteria by means of suitable vectors.

According to another aspect, the present invention relates to an antibody protein characterised in that it is specific for a polypeptide according to the invention. Therefore, the antibody protein according to the invention binds to an epitope of OTRPC4 or to an epitope of one of the alternative forms described above.

For numerous applications of the antibodies according to the invention, it is desirable to have as few antigen-binding, i.e. OTRPC4-binding units as possible. Therefore, in another preferred embodiment, an antibody protein according to the invention is a fragment antigen-binding (Fab) fragment. These OTRPC4 specific antibody proteins according to the invention consist of the variable regions of the two chains which are held together by the adjacent constant region. These may be formed by protease digestion, e.g. with papain, from conventional antibodies, but similar Fab fragments may also be prepared in the meantime by genetic engineering. In another preferred embodiment, an antibody protein according to the invention is an F(ab′)₂ fragment which can be produced by proteolytic cleaving with pepsin.

Using genetic engineering methods, it is possible to produce shortened antibody fragments consisting only of the variable regions of the heavy (VH) and light chain (VL). These are referred to as Fv fragments (in English: fragment variable). In another preferred embodiment, an OTRPC4-specific antibody molecule according to the invention is an Fv fragment of this kind. Since these Fv fragments lack the covalent linking of the two chains by the cysteines of the constant chains, the Fv fragments are often stabilised. It is advantageous to link the variable regions of the heavy and light chain by means of a short peptide fragment, e.g. 10 to 30 amino acids, preferably 15 amino acids. This produces a single peptide strand of VH and VL linked by a peptide linker. An antibody protein of this kind is referred to as an Fv single chain or single-chain-Fv (scFv). Examples of scFv antibody proteins of this kind known from the art are described in Huston et al. (1988, PNAS 16: 5879-5883). Therefore, in yet another preferred embodiment, an OTRPC4-specific antibody protein according to the invention is a single-chain Fv protein (scFv).

In recent years, various strategies have been developed for preparing scFv as multimeric derivatives. This is supposed to lead, in particular, to recombinant antibodies with improved pharmacokinetic and biodistribution properties and with enhanced binding affinities. In order to achieve multimerisation of the scFv, scFv have been prepared as infusion proteins with multimerisation domains. The multimerisation domains used have been, for example, the CH3 region of an IgG or coiled coil structure (helix structure) such as leucine zipper domains. However, there are also strategies in which the interaction between the VHNL regions of the scFv have been used for multimerisation (e.g. di-, tri- and pentabodies). Therefore, in another preferred embodiment, an antibody protein according to the invention is a diabody antibody fragment which is specific for an OTRPC4 epitope. The term diabody in the art refers to a bivalent homodimeric scFv derivative (Hu et al., 1996, PNAS 16: 5879-5883). The shortening of the linker in an scFv molecule to 5-10 amino acids results in the formation of homodimers in which there is an inter-VH/VL chain conglomeration. Diabodies may additionally be stabilised by the incorporation of disulphide bridges. Examples of diabody antibody proteins from the prior art may be found in Perisic et al. (1994, Structure 2: 1217-1226). The term minibody in the art denotes a bivalent, homodimeric scFv derivative. It consists of a fusion protein which contains the CH3 region of an immunoglobulin, preferably IgG, most preferably IgG1 as the dimerisation region which is connected to the scFv via a hinge region (e.g. IgG1 again) and a linker region. The disulphide bridges in the hinge region are usually formed in higher cells and not in prokaryotes. In another preferred embodiment, an antibody protein according to the invention is an OTRPC4-specific minibody antibody fragment. Examples of minibody antibody proteins in the prior art can be found in Hu et al. (1996, Cancer Res. 56: 3055-61).

By triabody is meant a trivalent homotrimeric scFv derivative (Kortt et al. 1997 Protein Engineering 10: 423-433). ScFv derivatives in which VH-VL are fused directly without a linker sequence lead to the formation of trimers.

By tetravalent miniantibody the skilled person understands a tetravalent homodimeric scFv derivative (Pack et al., 1995 J. Mol. Biol. 246: 28-34). The multimerisation is effected by means of tetrameric coiled coil domains.

Most preferably, an antibody protein according to the invention is totally human.

In another aspect, the present invention relates to a process for preparing an antibody protein according to the invention which comprises the following steps: a host selected from a eukaryotic or prokaryotic cell which contains one or more vectors having one or more nucleic acids specific for the antibody protein, is cultivated under conditions under which said antibody protein is expressed by said host cell and said antibody protein is isolated.

The antibody proteins according to the invention may also be prepared in a process according to the invention in fungi such as Pichia pastoris, Saccharomyces cerevisiae, Schizosaccharomyces, Trichoderma, with vectors which lead to intracellular expression or secretion. The process for preparing antibody proteins according to the invention may also be carried out using insect cells, e.g. as a transient or stable expression system or Baculovirus expression system, similar to that described above. Insect cell expression systems for the expression of antibody proteins are commercially available. Insect cell expression systems are particularly suitable for the scFv fragments according to the invention and Fab- or F(ab′)2 fragments and antibody proteins or fragments thereof which are fused to effector molecules, but also for complete antibody molecules.

The advantage of mammalian expression systems is that they produce very good glycosylation and folding conditions, e.g. transient expression systems such as in COS cells or stable expression systems e.g. BHK-, CHO- and myeloma cells. Mammalian cells may also be used, for example, with viral expression systems such as Vaccinia, Semliki-Forest virus and Adenovirus. Transgenic animals such as cows, goats and mice are also suitable for a process according to the invention. Transgenic plants such as Nicotiana tabacum (tobacco) may also be used in a process according to the invention. After genomic integration of the nucleic acid according to the invention which codes for an antibody protein according to the invention and is fused to a signal sequence, the antibody protein may be secreted into the interstitial space. Preparation using prokaryotic expression systems such as Escherichia coli, Bacillus subtilis, Streptomyces or Proteus mirabilis is preferably suitable for antibody fragments according to the invention such as Fab-F(ab′)2-, scFv-fragments, minibodies, diabodies and multimers of said fragments. The antibody proteins according to the invention may be prepared in a process according to the invention either intracellularly, e.g. in inclusion bodies, or by periplasmatic secretion in gram-negative bacteria using suitable vectors.

The invention also includes the use of a polypeptide according to the invention for finding blockers, activators or modulators of said polypeptides.

The word blockers means substances which inhibit the ion permeability of the channel by binding to the OTRPC4 cation channel itself, by binding to regulatory subunits or by interaction with the cell membrane or parts thereof.

The term activators means substances which stimulate the ion permeability of the channel by the OTRPC4 cation channel itself, by binding to regulatory subunits or by interaction with the cell membrane or parts thereof.

The term modulators denotes substances which modify the ion permeability of the channel, e.g. alter the selectivity of the channel with regard to calcium and sodium, by binding to the OTRPC4 cation channel itself, by binding to regulatory subunits or by interaction with the cell membrane or parts thereof. Blockers, activators or modulators can develop their pharmacological properties as a function of physical influences such as the pH, temperature and ion concentrations of the intra- or extracellular medium or as a function of the state of activation of the channel.

The invention also includes the use of a host according to the invention for finding blockers, activators or modulators of OTRPC4 channels.

According to another preferred aspect, the invention relates to a process for finding blockers, activators or modulators of OTRPC4, characterised in that a host according to the invention is incubated with a test substance.

According to another particularly preferred aspect, the invention relates to a process according to the invention, characterised in that a membrane current is measured, said membrane current is compared with a membrane current which is measured in said host after incubation with a known control substance or in the absence of the test substance. A process of this kind is described by way of example in Example 1, FIG. 7 of the invention.

According to another most particularly preferred aspect, the invention relates to a process in which said activator is bound to a channel, said host is incubated with a test substance and the displacement of the activator bound to the channel by the test substance is measured.

According to another most particularly preferred aspect, the invention relates to a process in which a host according to the invention is incubated with a test substance, the intracellular quantity of a divalent cation is determined and said quantity of divalent cation is compared with the quantity of said divalent cation which is measured when said host is incubated with a known control or in the absence of the test substance. A process of this kind is described in Example 1 of the invention, by way of example.

According to another most particularly preferred aspect, the invention relates to a process which is a high throughput screening (HTS) test or an ultrahigh throughput screening (UHTS) test. HTS within the scope of the invention relates to an experimental process in which a large number of test substances are tested simultaneously. An HTS process is preferably carried out in microtitre plates, partly or fully automated and connected to electronic equipment such as computers for data storage, analysis and interpretation using bioinformatics. Preferably, the system is automated by the use of robots which are able to handle a large number of microtitre plates at the same time and can perform several thousand tests per day. Preferably, a test substance is tested for a desired activator, blocker or modulator function in a cell based system with a cell according to the invention. The expression HTS also includes ultrahigh throughput screening tests (UHTS). Preferably, these UHTS processes are carried out using 384 or 1536 well microtitre plates, submicroliter and subnanoliter pipettors, improved plate reading equipment and procedures for preventing evaporation. HTS processes are described by way of example in U.S. Pat. Nos. 5,876,946 A and 5,902,732 A. The average skilled person can adapt the processes described above and in the Examples to an HTS or UHTS format without any inventive input.

An HTS for identifying blockers, activators or modulators of the OTRPC4 channel may be carried out as described in Example 1 but may also be carried out with so-called inducible expression systems, e.g. a plasmid which is inducible with tetracycline (Gossen, M. et al., Curr Opin Biotechnol, 1994, 5:516-20) or a system which is inducible by means of the Ecdyson receptor (Invitrogen). These systems and others are commercially available.

According to another important aspect, the present invention relates to an activator of OTRPC4 which can be discovered using a process according to the invention.

According to another important aspect, the present invention relates to a blocker of OTRPC4 which can be discovered using a process according to the invention.

According to another important aspect the present invention relates to a modulator of OTRPC4 which can be discovered using a process according to the invention.

Another preferred embodiment of the invention relates to an anti-sense nucleic acid, characterised in that it is capable of hybridising with part of a nucleic acid according to the invention under stringent conditions.

The term anti-sense nucleic acid (or anti-oligonucleotide) denotes DNA or RNA molecules for the purposes of the invention which are complementary with at least part of an mRNA molecule according to the invention, i.e. one which codes for an OTRPC4 polypeptide or fragment. A definition of anti-sense nucleic acid can also be found in the prior art (Weintraub H M, 1990 Scientific American, 262:34-40). In the cell, anti-sense nucleic acid molecules hybridise with the corresponding mRNA and form a double-stranded molecule. The anti-sense nucleic acids according to the invention interfere with the translation of the mRNA coding for OTRPC4 polypeptide or an OTRPC4 fragment, as the cell will not translate said double-stranded mRNA. The central region of the anti-sense nucleic acid within the scope of this invention contains at least 14 nucleotides which are complementary to the target RNA. The invention also includes peptide nucleic acids, phosphodiester anti-sense nucleic acids and phosphothioate oligonucleotides which are complementary to at least part of an mRNA molecule according to the invention coding for an OTRPC4 polypeptide or fragment. Substances of this kind which are specific for other target RNA are known from the prior art (Boado, R. J. et al., 1998, J. Pharm. Sci. 87:1308-1315).

In Example 1 (Table 1) five anti-sense sequences are mentioned by way of example and the criteria which led to the selection of these sequences are set out.

Another preferred embodiment of the invention relates to an anti-sense nucleic acid according to the invention which is a ribozyme. Ribozyme for the purposes of this invention is an RNA molecule which is capable of interacting specifically with the target RNA, i.e. the mRNA according to the invention coding for an OTRPC4 polypeptide or fragment, and is capable of irreversibly cutting it at a specific site. Preferably, the ribozyme according to the invention has a central sequence which is not complementary to the target RNA and is responsible for the catalytic activity thereof (catalytic region (a)) and two flanking sequences which are substantially complementary to two adjacent sequences of the target RNA (hybridisation region (b)), thus allow the binding of the ribozyme by base pairing and consequently the selective cleaving of the target RNA. A preferred embodiment of the ribozyme according to the invention can be represented by the following general formula:

| | |
|---|---|
| (b) (a) | (b) |
| 5′ [N₃₋₂₀][CUGANGARN₀₋₃₀SGAAA] | [N₃₋₂₀]3′, |

wherein N is a G, C, A or U, R is a purine and S is a pyrimidine and wherein the central region N₀₋₃₀ of sequence (a) can be replaced by a linker which is not a nucleic acid, namely a hydrocarbon chain, for example (see Thomson et al., 1993, Nucleic Acids Res 21:5600-5603). The ribozymes according to the invention may, for example, be a hammerhead, hairpin or axehead ribozyme. The structure of hammerhead ribozymes is known to those skilled in the art and is also described by way of example in Symons, R. H.

(1992, Ann. Rev. Biochem. 61:641-671) and Rossi, J. J. (1993, Methods 5:1-5). Hairpin ribozymes are capable of effectively cleaving target RNA in trans, the mechanism of activity being similar to the hammerhead ribozyme (see Rossi, supra, and Hampel et al., 1990, Nucleic Acids Res. 18:299-304). Axehead ribozymes are also capable of effectively cleaving in trans. They are described, for example, in Been, M. D. et al., (1994 Trends Biochem Sci. 19:251-256) and Wu, H. N. et al. (1993, Nucleic Acids Res. 21:4193-4199). Using the data known from the prior art, the skilled person can determine the minimum sequences and structure required for cleaving, and construct ribozymes which have the properties required for the purposes of the invention. The said ribozyme may also be modified within the scope of the invention in order to obtain increased nuclease resistance.

Examples of this are substitution of the 2′-OH groups of ribose by 2′-H, 2′-O-methyl, 2′-O-allyl, 2′-fluoro or 2′-amino groups (Paolella et al., 1992, and Pieken et al., 1991) or the modification of phosphodiester bonds, e.g. by exchanging one or two oxygen atoms for sulphur (phosphorus thioate and phosphorus dithioate; Eckstein, 1985 and Beaton et al., in: Eckstein, F. (Editor) Oligonucleotides and analogues—A practical approach—Oxford, JRL Press (1991), 109-135) or by a methyl group (methylphosphonate; Miller, loc. cit., 137-154). Other modifications include conjugating the RNA with poly-L-lysine, polyalkyl derivatives, cholesterol or polyethylamine glygol (PEG). Preferably, the ribozymes according to the invention contain at least one of the phosphate modifications described above and/or at least one of the ribose modifications described above.

According to another important aspect, the invention relates to a pharmaceutical composition which contains a nucleic acid according to the invention as well as pharmaceutically acceptable carriers or excipients.

According to another important aspect, the invention relates to a pharmaceutical composition which contains an anti-sense nucleic acid according to the invention as well as pharmaceutically acceptable carriers or excipients.

According to another important aspect, the invention relates to a pharmaceutical composition which contains a polypeptide according to the invention as well as pharmaceutically acceptable carriers or excipients.

Pharmaceutically acceptable carriers or excipients in this invention may be physiologically acceptable compounds which stabilise or improve the absorption of OTRPC4 activators, blockers or modulators, for example. Physiologically acceptable compounds of this kind include, for example, carbohydrates such as glucose, sucrose or dextranes, anti-oxidants such as ascorbic acid or glutathione, chelating agents, lower molecular compounds or other stabilisers or excipients (see Remington's Pharmaceutical Sciences, 18^{th} Edition, Mack Publ., Easton). The skilled person knows that the choice of a pharmaceutically acceptable carrier depends on the route of administration of the compound, for example.

According to another important aspect, the invention relates to a pharmaceutical composition which contains a vector according to the invention as well as pharmaceutically acceptable carriers or excipients. This pharmaceutical composition may also contain a vector according to the invention for gene therapy and may additionally comprise as an adjuvant a colloidal dispersion system or liposomes for a targeted administration of the pharmaceutical composition.

According to another important aspect, the invention relates to a pharmaceutical composition which contains a host according to the invention as well as pharmaceutically acceptable carriers or excipients. A host or a host cell which contains a vector according to the invention may also be used in a pharmaceutical composition within the scope of this invention, e.g. for gene therapy.

One example of a targeted system of administration, e.g. for anti-sense oligonucleotides or ribosomes according to the invention is said colloidal dispersion system. Colloidal dispersion systems comprise macromolecule complexes, nanocapsules, microspheres and lipid-based systems including oil-in-water emulsions, micelles, mixed micelles and liposomes or liposome formulations. Liposomes are the preferred colloidal system according to the invention. Liposomes are artificial membrane vesicles which are useful as carriers in vitro and in vivo. These formulations may carry a cationic, anionic or neutral charge. It has been shown that large unilamellar vesicles (LUV) ranging from 0.2-4.0 μm in size may enclose a major part of an aqueous buffer solution with large macromolecules. RNA, DNA and intact virions can be encapsulated in the aqueous phase inside and transported to the target in a biologically active form (Fraley, R. et al., 1981, Trends Biochem Sci. 6:77-80). In addition to mammalian cells, liposomes have also proved suitable for the targeted transporting of nucleotides into plant, yeast and bacterial cells. In order to be an efficient gene transfer carrier, the following properties should be present: (1) the genes should be enclosed with high efficiency without reducing their biological activity; (2) there should be preferential and substantial binding to the target cell compared with non-target cells; (3) the aqueous phase of the vehicle should be transferred highly efficiently into the target cell cytoplasm; and (4) the genetic information should be expressed accurately and efficiently (Mannino, R. J. et al., 1988, BioTechniques 6:682-690).

The composition of the liposomes usually consists of a combination of phospholipids, particularly, high phase transition temperature phospholipids, e.g. combined with steroids such as cholesterol. Other phospholipids or other lipids may also be used. The physical characteristics of the liposomes depend on the pH, the ion concentration and the presence of divalent cations.

The pharmaceutical composition according to the invention may also contain a vector according to the invention as a naked “gene expression vector”. This means that the vector according to the invention is not associated with an adjuvant for targeted administration (e.g. liposomes, colloidal particles, etc.). A chief advantage of naked DNA vectors is the absence of any immune response caused by the vector itself.

The invention also relates to the use of a nucleic acid according to the invention for preparing a medicament for treating a disease selected from among diabetes, hyperlipidaemia, hyperproteinaemia, hypertension, stroke, renal insufficiency, shock and other pathophysiological conditions characterised by hyper- and hypoosmolarity. These are, for example, other pathophysiological conditions which are accompanied by an increase or decrease in extracellular osmolarity, e.g. shock of various origins such as cardiogenic, metabolic, septic or anaphylactic shock or shock caused by bums or polytrauma.

The term shock for the purposes of this invention denotes a pathophysiological condition which leads to a generalised severe reduction in tissue perfusion and consequent tissue damage.

The invention also relates to the use of an anti-sense nucleic acid according to the invention for preparing a medicament for treating a disease selected from among diabetes, hyperlipidaemia, hyperproteinaemia, hypertension, stroke, renal insufficiency and shock.

These are, for example, other pathophysiological conditions which are accompanied by an increase or decrease in extracellular osmolarity, e.g. shock of various origins such as cardiogenic, metabolic, septic or anaphylactic shock or shock caused by bums or polytrauma.

The invention also relates to the use of a vector according to the invention for preparing a medicament for treating a disease selected from among diabetes, hyperlipidaemia, hyperproteinaemia, hypertension, stroke, renal insufficiency and shock. These are, for example, other pathophysiological conditions which are accompanied by an increase or decrease in extracellular osmolarity, e.g. shock of various origins such as cardiogenic, metabolic, septic or anaphylactic shock or shock caused by bums or polytrauma.

The invention also relates to the use of a host according to the invention for preparing a medicament for treating a disease selected from among diabetes, hyperlipidaemia, hyperproteinaemia, hypertension, stroke, renal insufficiency and shock. These are, for example, other pathophysiological conditions which are accompanied by an increase or decrease in extracellular osmolarity, e.g. shock of various origins such as cardiogenic, metabolic, septic or anaphylactic shock or shock caused by bums or polytrauma.

Another essential aspect of the invention is a non-human mammal, characterised in that it contains a nucleic acid according to the invention (transgene) in addition to its genome. This refers to a non-human transgenic mammal which in addition to its genome has a nucleic acid sequence according to the invention coding for OTRPC4 or a fragment thereof stably integrated in some of its body cells (chimaera) or in all its body cells and which expresses OTRPC4 polypeptide or a fragment. The skilled person is familiar with transgenic mammals which are transgenic for other sequences (see Schenkel, J., Spektrum Akad. Cerl., 1995). Transgenic mammals according to the invention include, for example, transgenic rodents such as rats, mice and hamsters but also goats, cattle, pigs and sheep and other non-human mammals known to the skilled person. Mice are particularly preferred. According to another essential aspect the invention relates to a non-human mammal, characterised in that a nucleic acid according to the invention is inactivated in its genome (gene knock-out). By this is meant a non-human so-called knock-out mammal in whose genome the endogenous nucleic acid sequence corresponding to a nucleic acid sequence according to the invention coding for OTRPC4 or a fragment thereof is inactivated and in which no or only small amounts of OTRPC4 polypeptide or fragment thereof are expressed. Small amounts mean that the expression of OTRPC4 polypeptide or fragment is reduced by at least 50%, preferably at least 50 to 80%, most preferably at least 80 to 100%, compared with comparable non-knock-out mammals. The inactivation is often achieved by cloning-in a reporter sequence, e.g. the gene for neomycin resistance. The skilled person is familiar with other knock-out mammals in which other sequences are inactivated. Knock-out mammals according to the invention include, for example, knock-out rodents such as rats, mice and hamsters as well as goats, cattle, pigs, sheep and other non-human mammals known to those skilled in the art. Mice are particularly preferred. Processes for producing a knock-out mammal according to the invention are described hereinafter. The construction of a recombinant vector for a conditional knock-out is described by way of example in Example 1.

According to another essential aspect, the invention relates to a non-human mammal, characterised in that a nucleic acid according to the invention is modified in its genome (gene knock-in). This modification can be achieved by homologous recombination of the coding nucleic acid and causes an OTRPC4 polypeptide or fragment thereof with modified properties to be expressed in this mammal, for example. This can be done, for example, by mutation in a small part of the coding nucleic acid. Examples of knock-in mammals according to the invention include knock-in rodents such as rats, mice and hamsters but also goats, cattle, pigs, sheep and other non-human mammals known in the art. Mice are particularly preferred. Processes for producing a knock-in mammal according to the invention are described hereinafter.

The non-human transgenic or knock-out or knock-in mammals according to the invention are exceptionally suitable for analysing the function of the OTRPC4 gene or polypeptide. The mammals according to the invention can be compared with mammals of the same species or advantageously of the same litter (litter mates) and in this way the function of the polypeptide according to the invention can be investigated.

The invention also includes processes for producing a non-human mammal, characterised in that

(a) embryonic stem cells of said non-human mammal are transfected with a vector which contains a nucleic acid according to the invention and allows recombination between the genomic DNA, said non-human mammal and the nucleic acid contained in the vector;

(b) stably transfected stem cells from step (a) are isolated and these are transferred into the germ line of a female animal of said non-human mammal; and

(c) the offspring of said female animal from step (b) with a male animal of the same species are analysed for animals which express the polypeptide coded by the nucleic acid from step a).

Embryonic stem cells (ES) can be obtained by cultivating the inner cell mass of blastocysts and multiplying them in tissue culture. For the purposes of the invention, differentiation of the stem cells is prevented by cultivating them on nutrient cells obtained from fibroblasts or by adding leukaemia inhibiting factor (LIF) to the culture medium. The incorporation of nucleic acid according to the invention in ES cells, for example DNA coding for OTRPC4 or a fragment thereof, is carried out for example by transfection, retrovirus infection or electroporation. A vector of this kind carries, for example, the neomycin gene which confers resistance to G418. In this way, successfully transfected embryonic stem cells can be identified by adding G418 to the culture medium. Only successfully transfected ES are able to grow under these conditions. Transfected ES of this kind are transferred back into blastocysts, for example, and these are transferred into the germ line of a female mammal according to the invention. The mutated cells are integrated into the developing embryo and participate in the development of all the tissues. In this way the transgene according to the invention enters the germ line. Chimaeric animals are formed which may be characterised, for example, by the previous selection of ES cells and receptor blastocysts of animals of a different skin colour. By multiple cultivation of the chimeric animals, homozygotic animals are obtained which express the transgene in every tissue.

Another process according to the invention for producing non-human transgenic mammals comprises isolating fertilised egg cells, microinjecting nucleic acid according to the invention coding for OTRPC4 or a fragment thereof, implanting said fertilised egg cells in the germ line of a pseudo-pregnant female animal of said non-human mammal and investigating the offspring of said female animal with a male animal of the same species for expression of the transgene.

The nucleic acid, preferably DNA, introduced by microinjection is often integrated at a different place from the comparable endogenous nucleic acid but is usually expressed in exactly the same way. Said nucleic acid according to the invention may be integrated in the genome in one, or in multiple, e.g. two to several hundred or thousand copies. Details of methods for producing transgenic non-human mammals are known to the skilled person (see Schenke, J., Spektrum Akad. Verl., 1995).

The invention also includes processes for producing a non-human mammal, characterised in that

(a) embryonic stem cells of said non-human mammal are transfected with a vector which contains a nucleic acid which is capable of hybridising with a nucleic acid according to the invention under stringent conditions, and is inactivated by the insertion of an additional nucleic acid sequence, and allows recombination between the genomic DNA of said non-human mammal and the nucleic acid contained in the vector;

(b) stably transfected stem cells from step (a) are isolated and these are transferred into the germ line of a female animal of said non-human mammal; and

(c) the offspring of said female animal from step (b) with a male animal of the same species are analysed for animals which express the polypeptide coded by the nucleic acid from step (a).

In order to produce a mammal according to the invention in which the endogenous gene which corresponds to an OTRPC4 nucleic acid sequence according to the invention or comprises such a sequence is inactivated by so-called knock-out, the gene is controlled by homologous recombination and inactivated. By homologous recombination is meant processes which make it possible to incorporate nucleic acid, e.g. DNA, in genes in a controlled manner. A cloned copy of the endogenous gene is replaced by a functionless copy. For example, the copy incorporated is interrupted by an inserted copy of one or more antibiotic resistance genes, leading to inactivation. For example, the sequence for the target gene may be interrupted by the neomycin resistance gene. By introducing Herpes simplex virus thymidine kinase (HSV-tk) at the end of the construct, for example, it is possible to identify those cells in which homologous recombination has taken place.

Within the scope of the invention, an inactivated copy of a nucleic acid coding for OTRPC4 or a fragment thereof, cloned into a suitable vector, is incorporated in embryonic stem cells (as described above) by a suitable method, i.e. by transfection, retrovirus infection or electroporation into the ES. The incorporated nucleic acid enters into homologous recombination in part of the ES with the corresponding cellular copy of the OTRPC4 gene and replaces the gene with the nucleic acid according to the invention which has been inserted. For example, using the antibiotic G418 and the antiviral substance ganciclovir it is possible to identify those ES in which homologous recombination has taken place. ES in which homologous recombination has taken place are injected into a blastocyst which is inserted in the uterus of a female, non-human mammal of the same species as the ES. Chimaeric animals are produced which can be characterised, for example, by a previous selection of ES cells and receptor blastocysts of animals of different skin colour. By multiple cultivation of the chimaeric animals, homozygotic animals are obtained in which the target gene is totally inactivated in every tissue.

The invention also includes processes for producing a non-human mammal, characterised in that

(a) embryonic stem cells of said non-human mammal are transfected with a vector which contains a nucleic acid which is capable of hybridising with a nucleic acid according to the invention under stringent conditions, and is modified by insertion of an additional nucleic acid sequence, and allows recombination between the genomic DNA of said non-human mammal and the nucleic acid contained in the vector;

(b) stably transfected stem cells from step (a) are isolated and these are transferred into the germ line of a female animal of said non-human mammal; and

(c) the offspring of said female animal from step (b) with a male animal of the same species are analysed for animals which express the polypeptide coded by the nucleic acid from step (a).

The process for producing knock-in animals is carried out similarly to the process for producing knock-out animals, except that the target gene is not inactivated but modified.

The Example which follows is intended to aid the understanding of the invention and should not be regarded in any way as limiting the scope of the invention.

### Example 1

Structure of an OTRPC4 Channel

In the Example which follows, the cloning and structure of an OTRPC4 polypeptide or OTRPC4 cation channel according to the invention are described by way of example. The description or use of the term OTRPC4-DNA, OTRPC4-RNA, protein or channel should not be regarded as limiting the scope of the invention in any way but are intended only to illustrate the invention. Other OTRPC4-DNA, OTRPC4-RNA, proteins or channels are described in the specification.

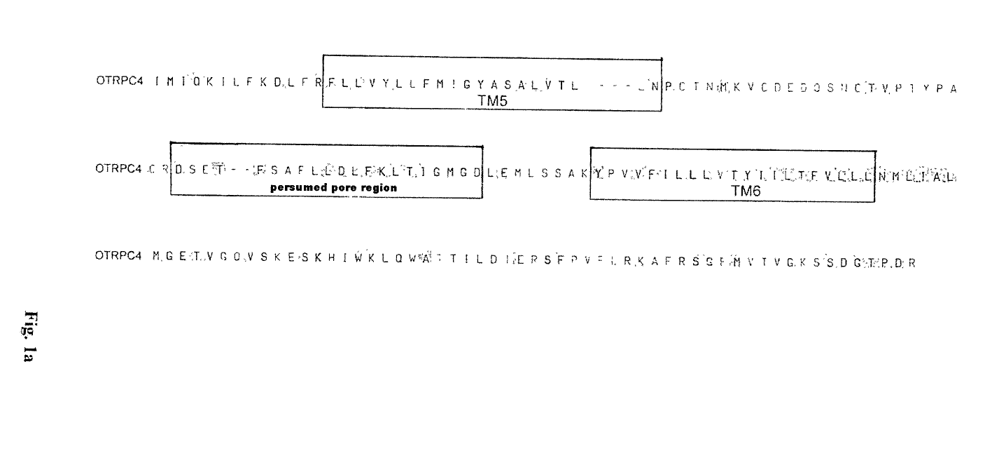

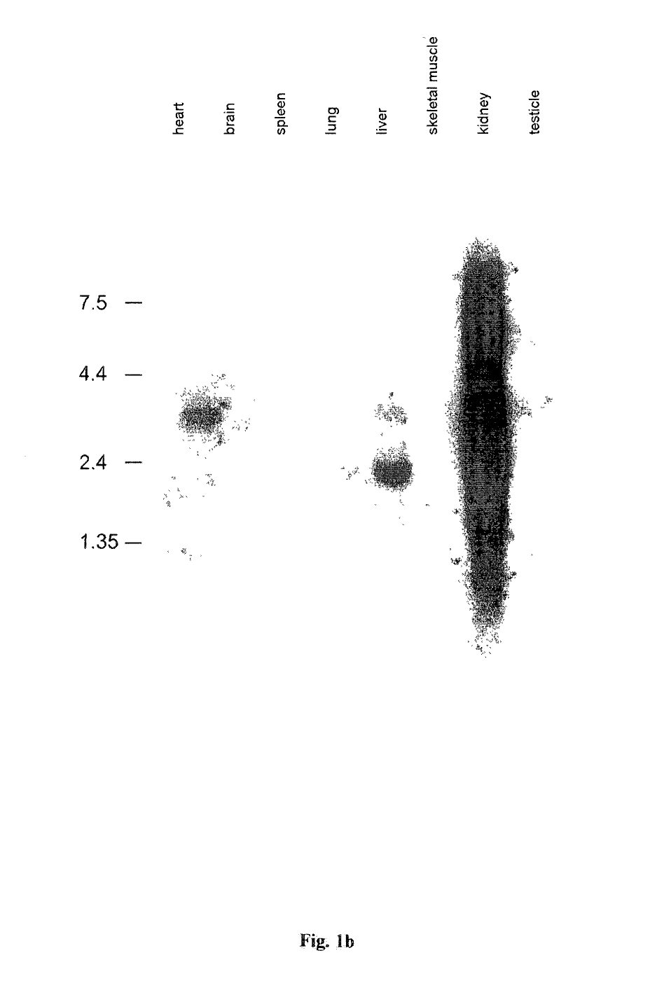

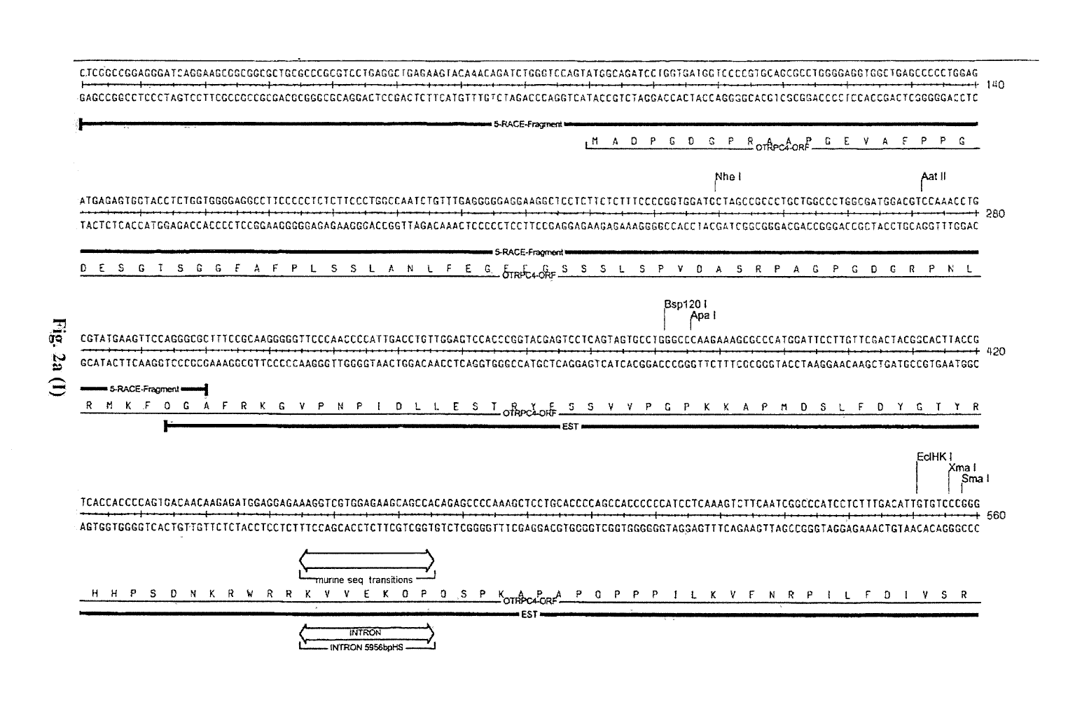

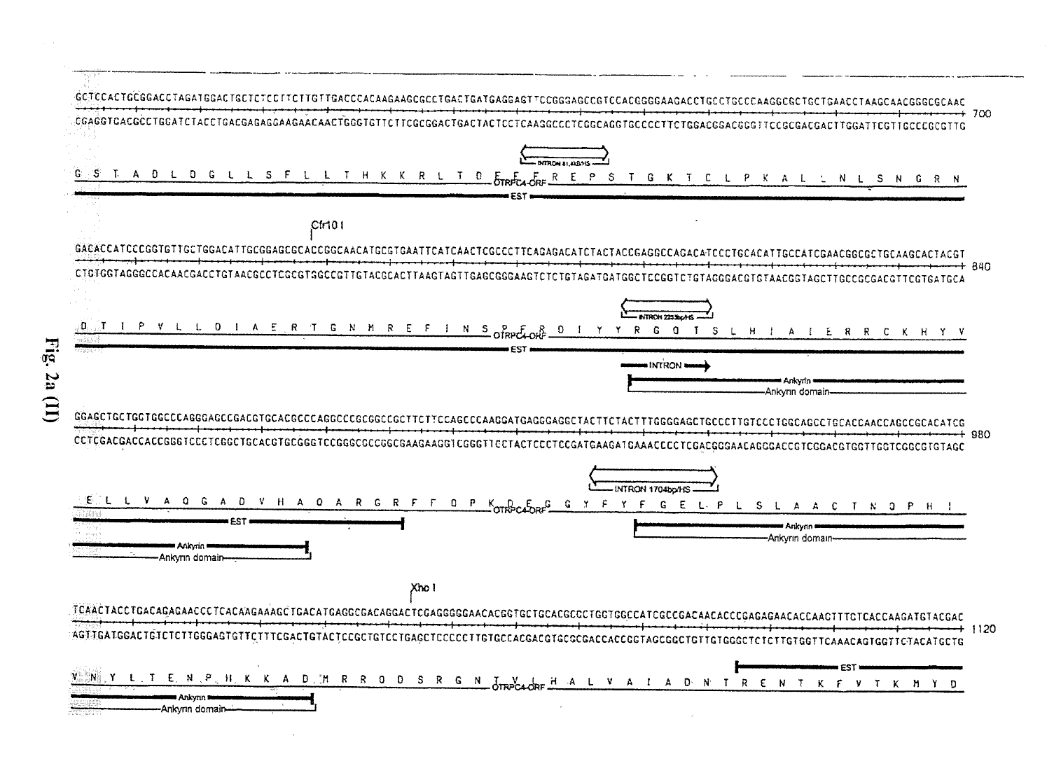

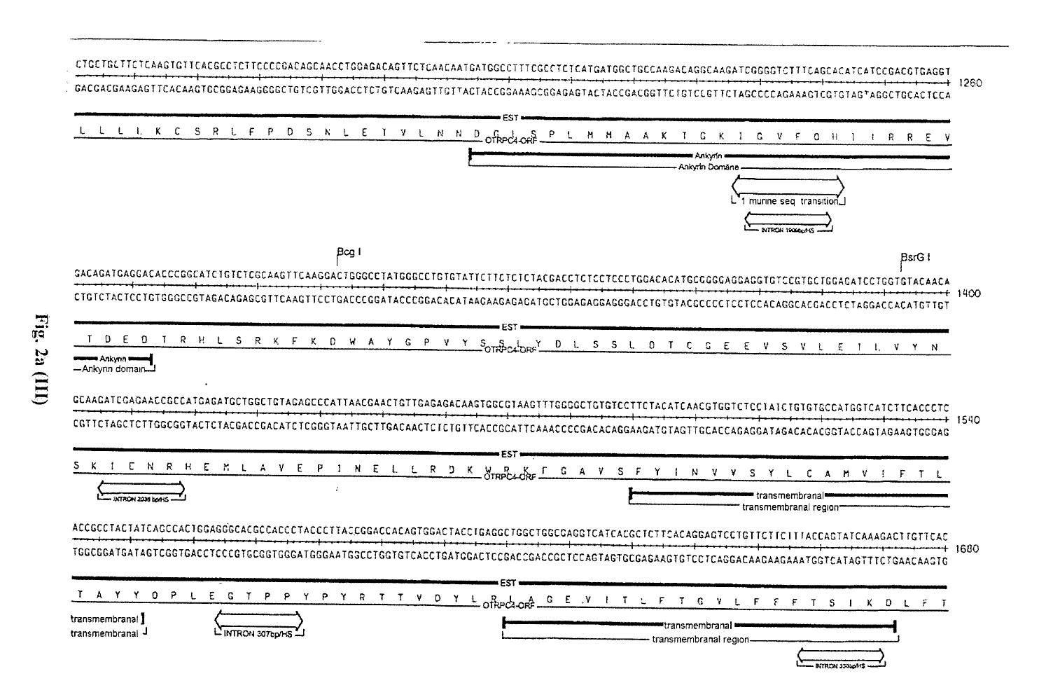

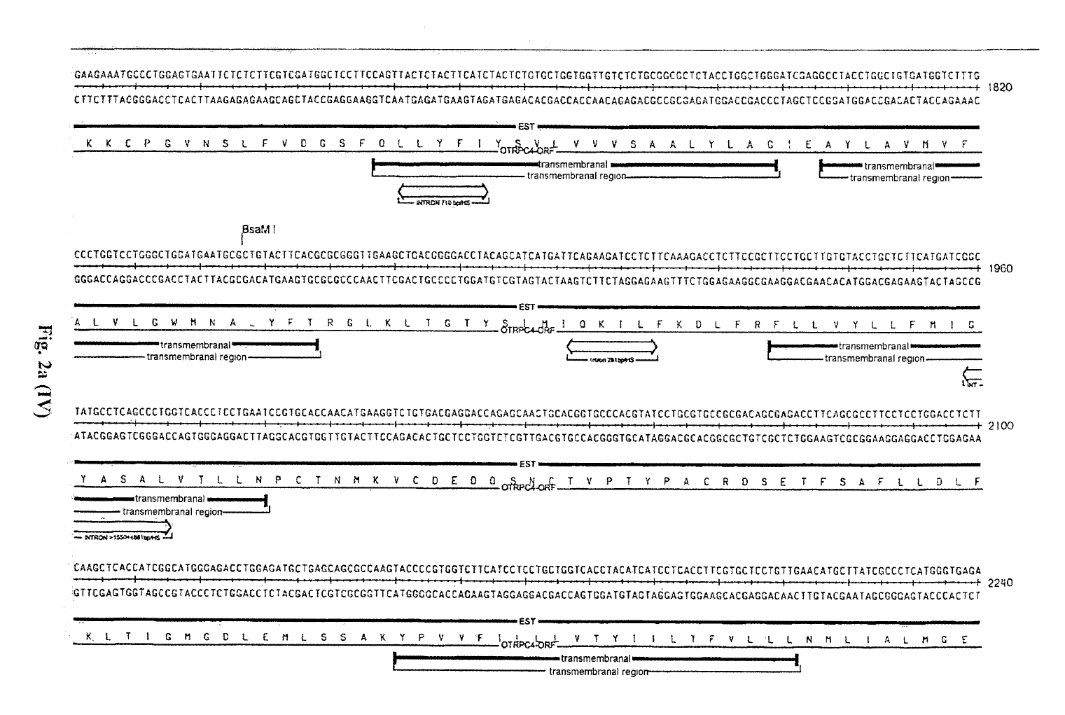

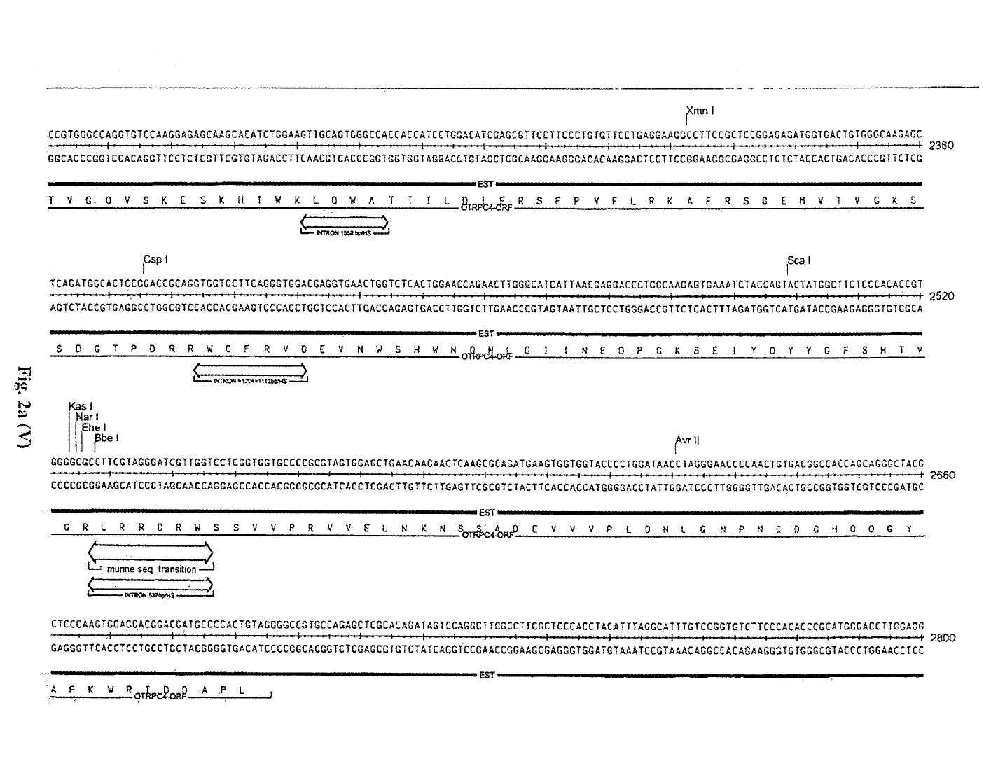

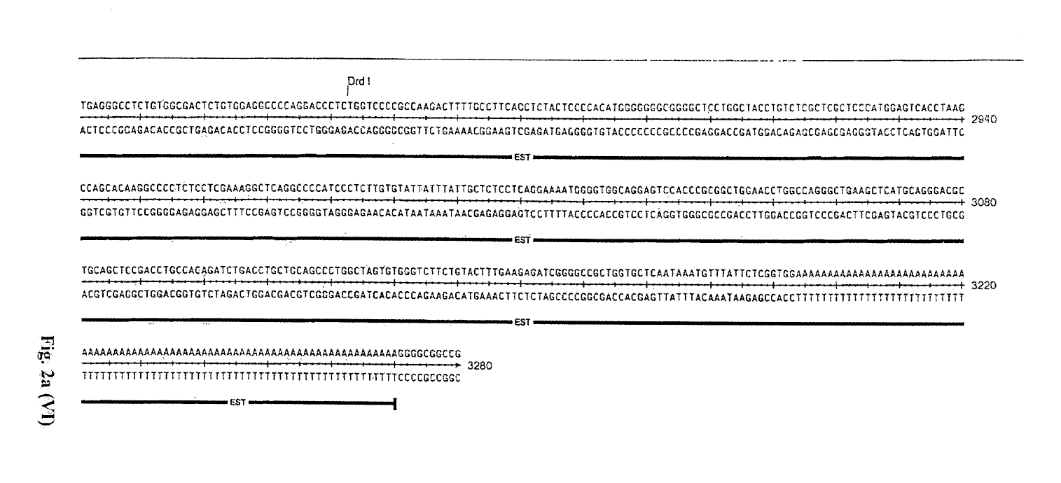

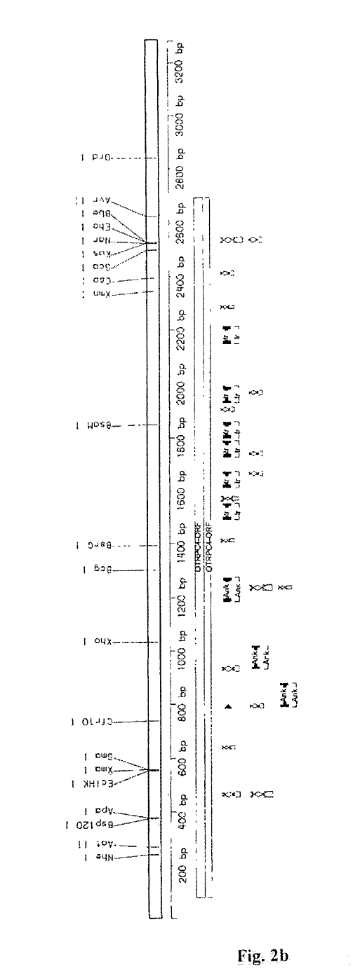

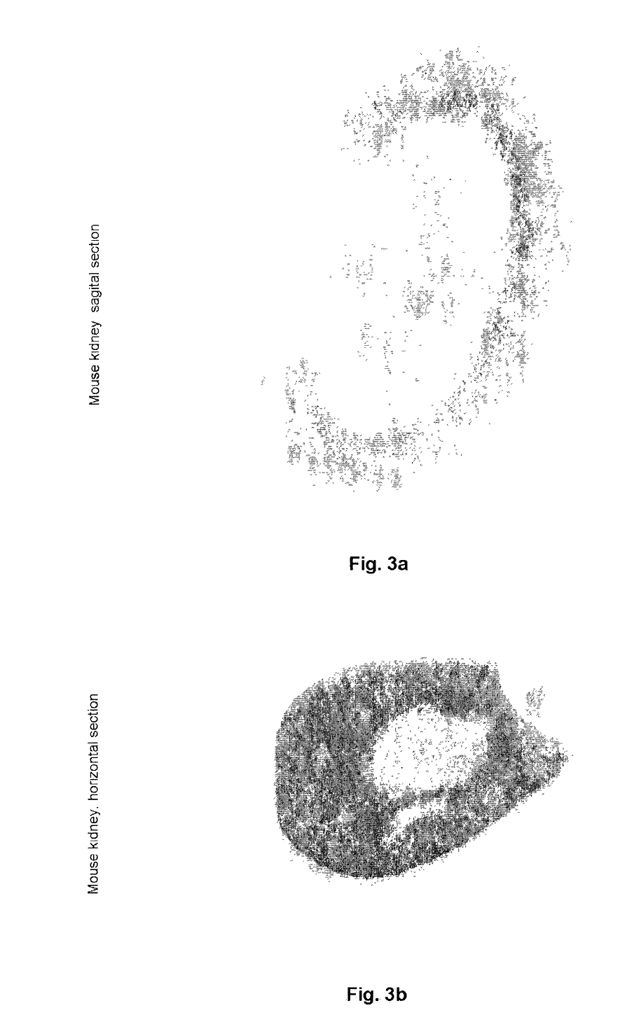

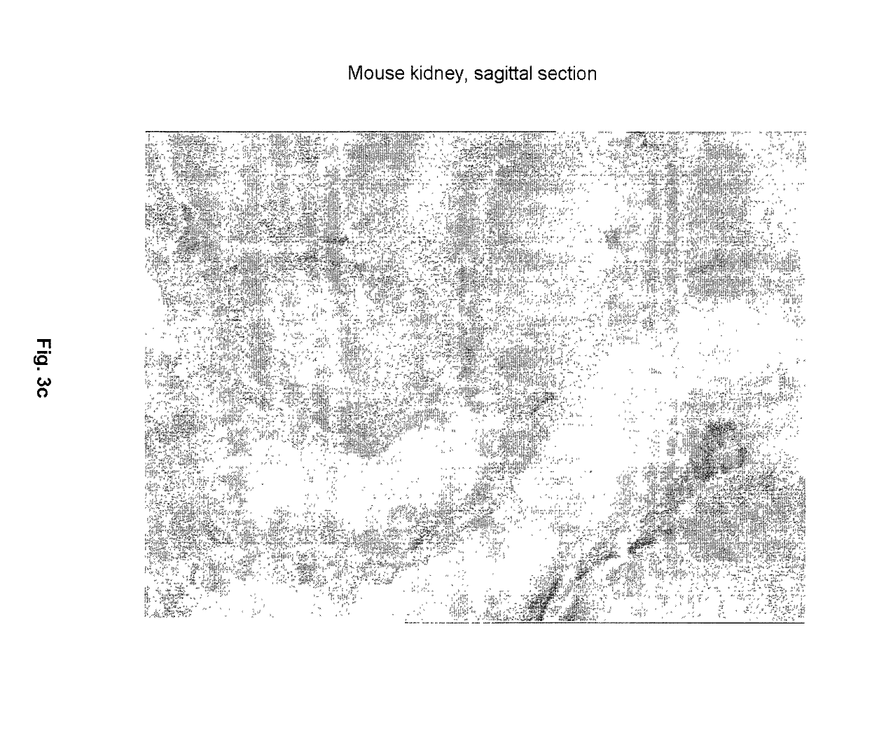

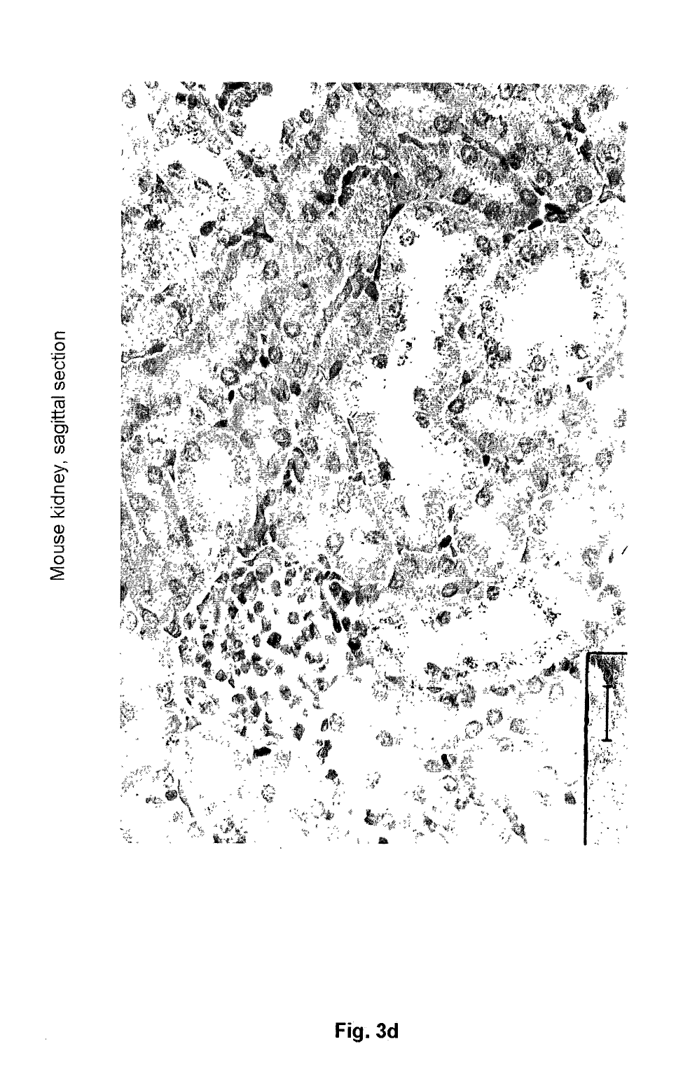

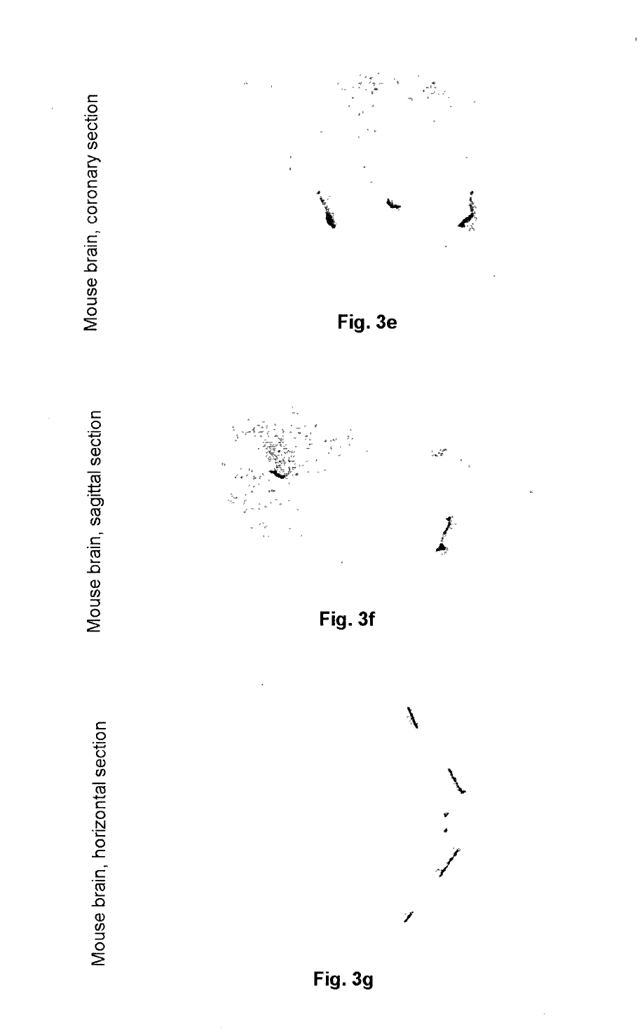

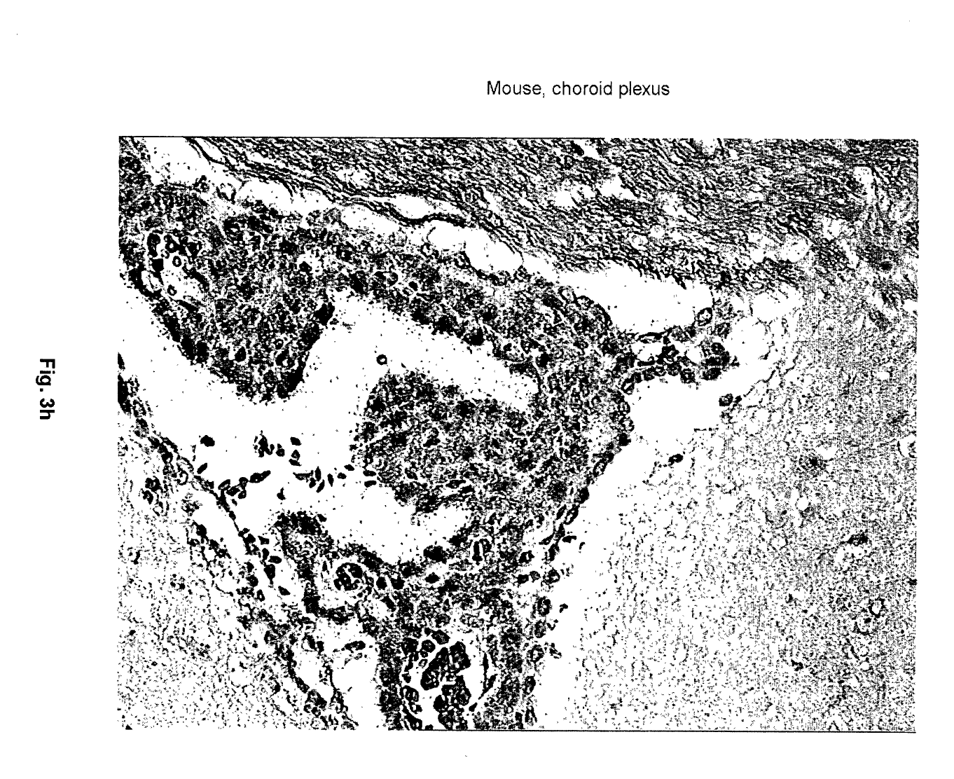

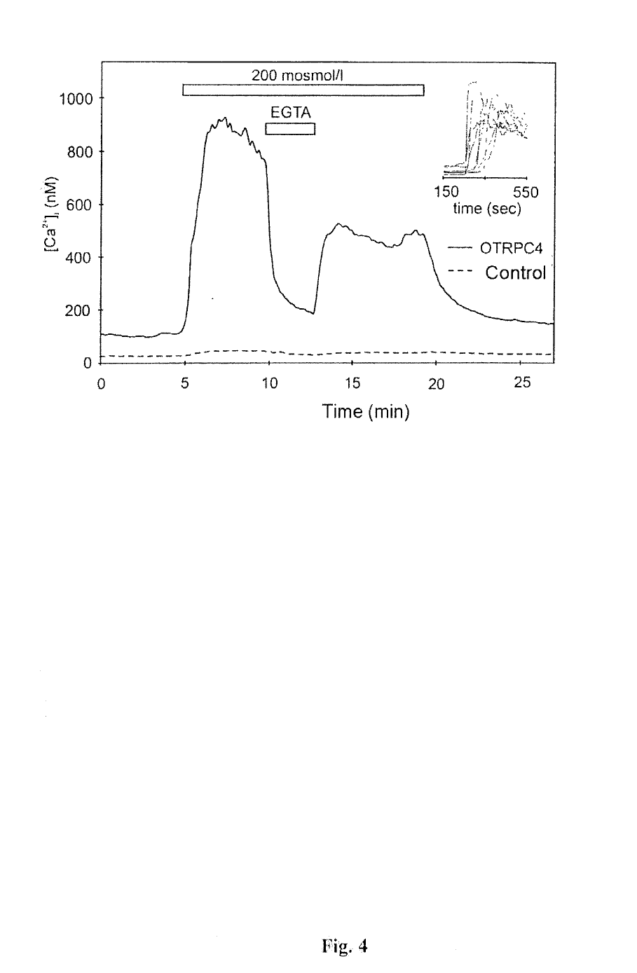

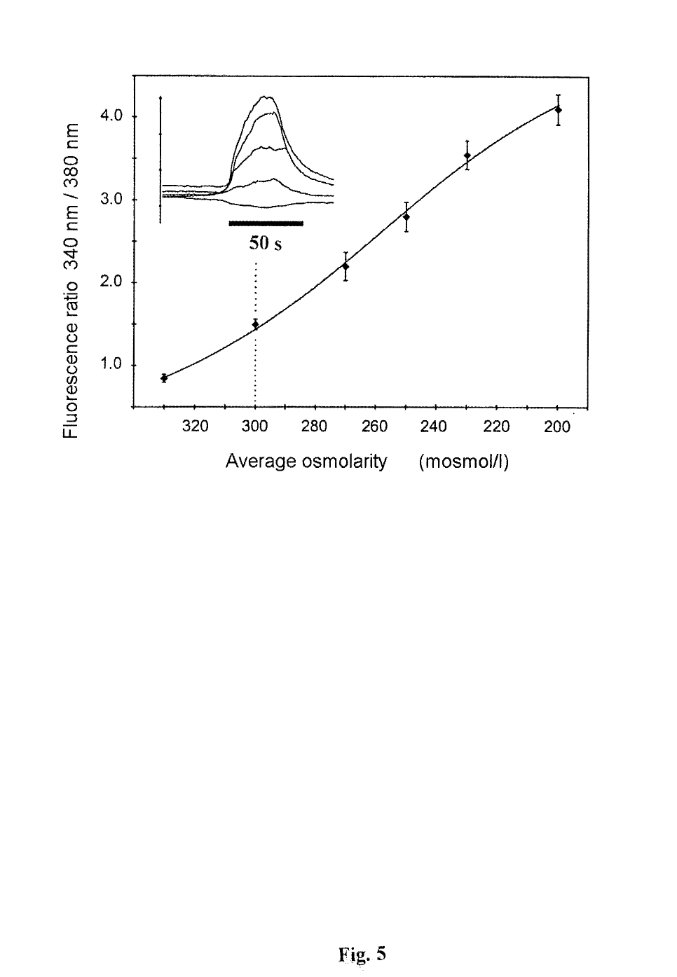

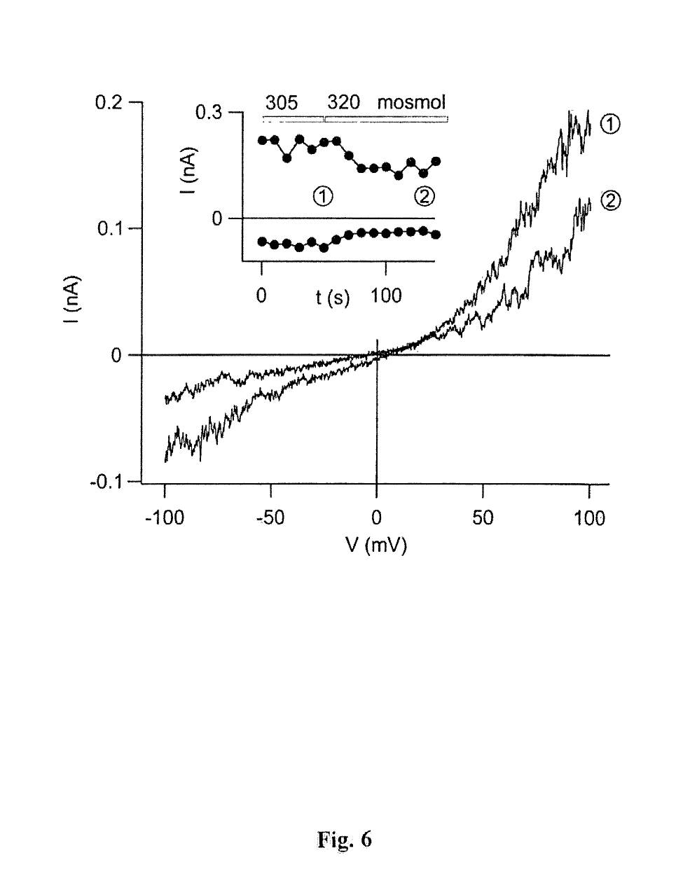

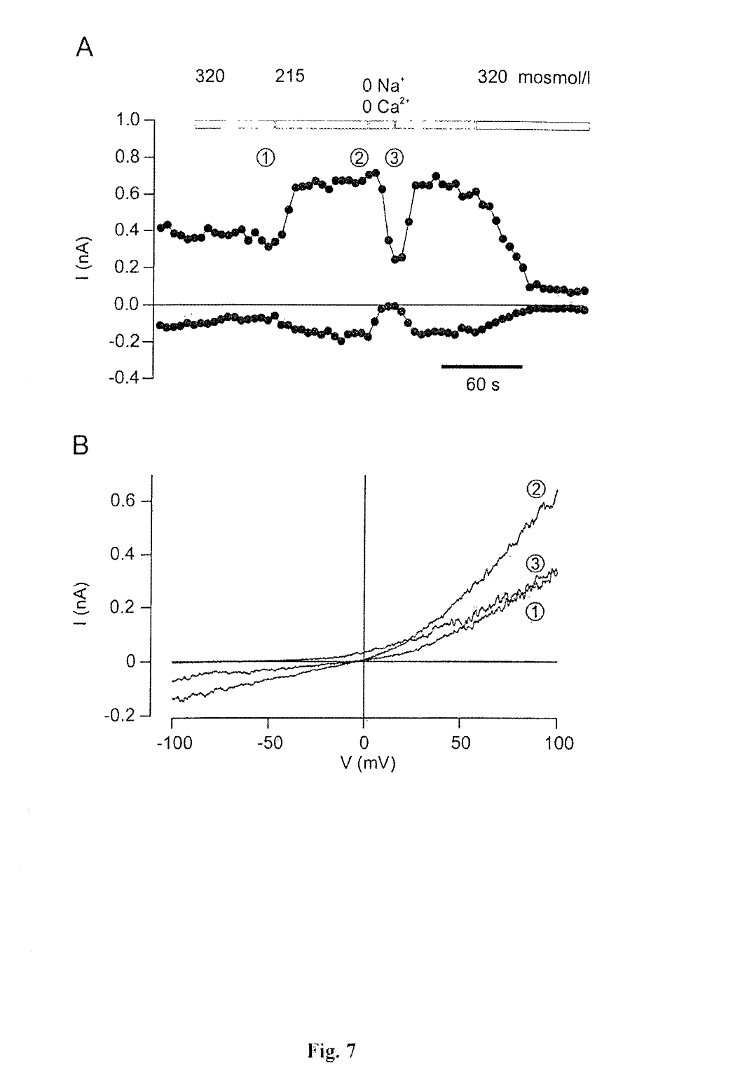

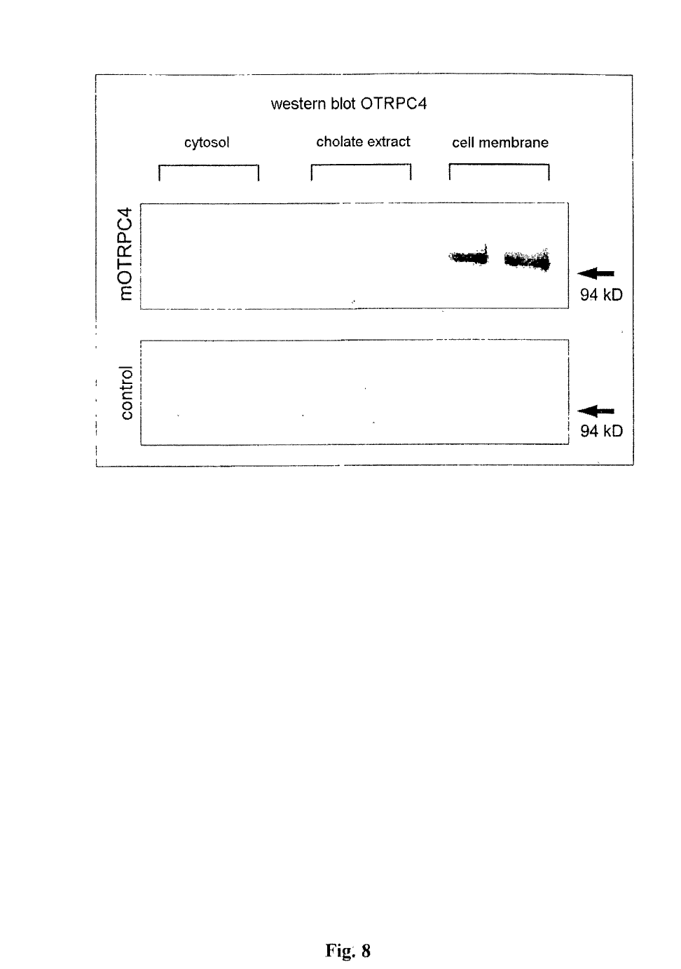

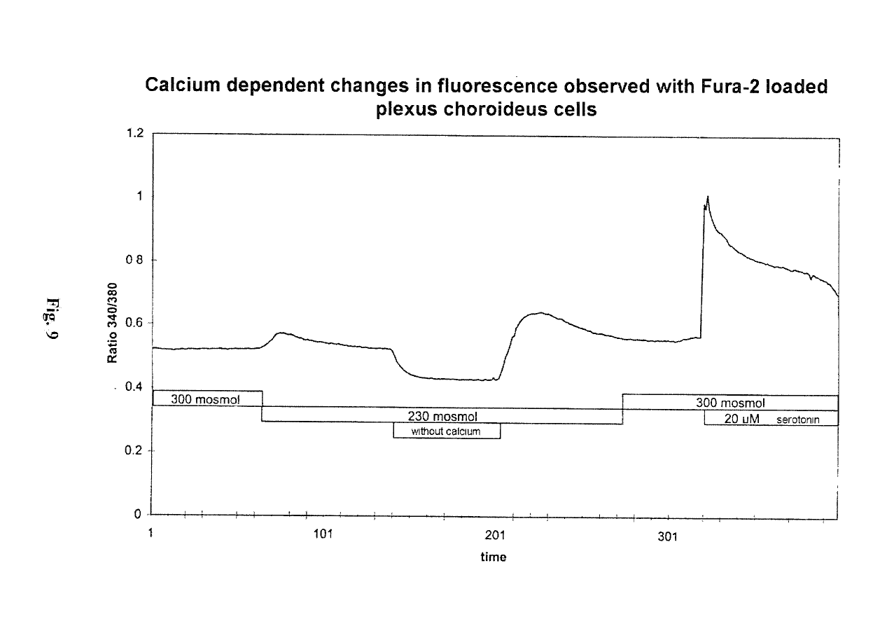

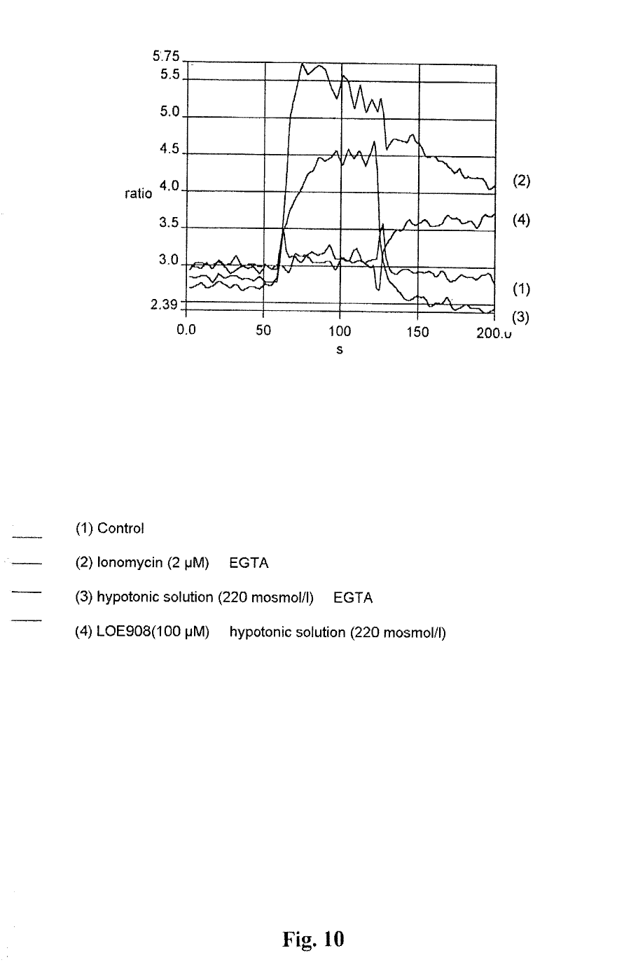

The mRNA expression was investigated by Northern blot hybridisations with expressed sequence tag (EST) fragments AA139413 and W53556 (deposited in Genbank—http://www.ncbi.nlm.nih.gov/Genbank/). An RNA transcript 3.3 kb long expressed primarily in the liver, heart, kidneys and testis, was identified (FIG. 1b). From the RNA purified from a mouse kidney, a cDNA 3277 bp long was cloned using the RACE-PCR method, containing an open reading frame of 2616 bp (SEQ ID NOS:7 and 8). The genomic organisation of the murine sequence of OTRPC4 was clarified by sequencing the intron-exon transitions and is shown by way of example in FIG. 2. Hybridisations in situ with a fragment from the coding region of OTRPC4-DNA showed a high expression of OTRPC4-RNA in the distal convoluted renal tubules but also in choroid plexus of the ventricles of the brain (FIG. 3).

The cDNA of OTRPC4 was cloned into a eukaryotic expression plasmid containing, at the C-terminal end, a green fluorescent protein (GFP) fusion section (pEGFP-N1). This plasmid was used for the subsequent expression studies. It can be inferred from the nucleotide sequence that the OTRPC4 protein may consist of 871 amino acids, for example, and contains 6 possible transmembranal segments with a sequence between segments 5 and 6 which possibly codes for a pore region of a channel (FIG. 1a). After transient transfection (which was carried out using the FuGENE 6 transfection reagent) of the expression plasmid coding for the OTRPC4-GFP fusion protein in HEK293 cells, dots of fluorescence could be detected in the plasma membrane 24-36 hours later. It was thus possible to demonstrate that the GFP fusion protein and hence the OTRPC4 channel protein is expressed and incorporated in the plasma membrane. For the following experiments, HEK293 cells were transiently transfected with the above-mentioned expression plasmid containing OTRPC4 and compared, after 24-36 hours, with untransfected control cells.

Increasing the Extracellular Calcium Concentration in HEK293 Cells which Express the OTRPC4 Channel

In order to study the function of the OTRPC4 channel, HEK293 cells were transiently transfected with expression plasmid containing the OTRPC4-GFP fusion construct and then the concentration of the intracellular calcium concentration ([Ca²⁺]ᵢ was measured using the FURA-2 method using a monochromatic single cell calcium measuring plate (FIG. 4). The basal [Ca²⁺]ᵢ in OTRPC4-expressing cells was significantly increased compared with the control cells (94±11 nM; 50 cells measured in three independent experiments versus 41±3 nM; 63 cells measured in three independent experiments). In order to demonstrate that the increase in [Ca²⁺]ᵢ is caused by an influx of extracellular calcium, so-called manganese quenching experiments were carried out showing that the FURA-2 signal is inhibited by the addition of 200 nM manganese to the extracellular solution. In addition, omitting the calcium from the extracellular solution resulted in an inhibition of the basally increased FURA-2 signal (see FIG. 4). The two results indicate that OTRPC4 is a calcium-permeable cation channel of the membrane.

The OTRPC4-Mediated Change in the Intracellular Calcium Concentration is Dependent on the Osmolarity of the Extracellular Solution

The influence of extracellular osmolarity on the channel activity of OTRPC4 was investigated. After reducing the osmolarity of the extracellular solution, there was a long, transient and reversible increase in the [Ca²⁺]ᵢ in the OTRPC4 expressing cells but not in the control cells (FIG. 5). Increasing the osmolarity of the extracellular solution, on the other hand, reduced the [Ca²⁺]ᵢ (cf. the small diagram in FIG. 4). A significant change in the [Ca²⁺]ᵢ was observed even when the extracellular solution was changed by 30 mosmol/l. The changes in [Ca²⁺]ᵢ triggered by changing the osmolarity of the extracellular solution occurred rapidly and reached a peak after about 30 seconds, but varied from cell to cell (see FIG. 4). After returning to the normoosmolar solution, [Ca²⁺]ᵢ rapidly returned to its base value. In order to distinguish between an influx of calcium from the extracellular medium and a release of calcium from intracellular calcium stores, calcium was replaced by EGTA in the extracellular medium while the cells were exposed to a hypotonic medium. Under these conditions, the FURA-2 signal returned to the base value (see FIG. 4). When the intracellular stores were emptied by previously adding thapsigargin (5 μM), an inhibitor of the calcium ATPase of the endoplasmatic reticulum (Foskett, J. K. in Cellular and Molecular Physiology of Cell Volume Regulation (ed. Strange, K.) 259-277 (CRC Press, Boca Raton, 1994), this did not alter the amplitude of the FURA-2 signal triggered by the hypotonic medium.

These two results prove that in the OTRPC4-expressing cells a channel is expressed in the membrane which is responsible for an osmotically regulated influx of calcium from the extracellular medium. Of the two lanthamides, Gd³⁺ and La³⁺, which block most of the calcium permeable cation channels (see Ref. 11, 14, 24-26), LaCl₃ inhibited the influx of calcium into OTRPC4-expression cells, triggered by hypoosmolarity, by about 50% at a concentration of 100 μM, whereas GdCl₃ showed no effect at a concentration of 1 mM. The members of the STRPC subfamily of the TRPC channels are activated by signals produced by the activation of the phospholipase C-β (PCL-β) (6-15). The activation of the endogenously expressed muscarinergic receptors and hence activation of the PLC-β in OTRPC4-expressing cells had no effect on the calcium influx in these cells.

The addition of capsaicin (10 μM) and resiniferatoxin (10 μM) as well as a brief increase in the temperature to 65° C. had no effect on cells which expressed OTRPC4.

Electrophysiological Characterisation of OTRPC4

Parallel to the increase in the basal [Ca²⁺]ᵢ cells which expressed OTRPC4 (detected by fluorescence of the GFP fusion portion) exhibited a basal ion flux measured in the so-called whole cell configuration. Because of the rapid “run-down” of the ion fluxes, the other experiments were carried out using the so-called perforated patch method. Measured in standard extracellular solution, the current-voltage curve, measured by applying voltage ramps, show an outwardly rectifying form with a reverse potential of about 0 mV (FIG. 6). When the Na⁺- and Ca²⁺-ions were removed from the extracellular solution, the inward flux disappeared and the reverse potential moved into the negative area. The average ion fluxes at −100 and +100 mV measured with the aid of ramp protocols were −12.8±1.1 and +32.2±2.7 pA/pF, respectively (n=17, Cₘ=9.6±5.5 pF). These values differ distinctly from the values measured in the control cells under the same conditions; the control cells showed only slight ion fluxes (−2.6±0.7 and +3.9±0.9 pA/pF, n=5, Cₘ=13.4±1.5 pF) with a non-linear current-voltage curve and an Eᵣ of −16±2.1 mV.

Replacing the extracellular standard solution with a solution which contained 100 mM NaCl and 100 mM mannitol (osmolarity: 320 mosmol/l) shifted Eᵣ to a more negative potential (−11.2±1.6 mV, n=14), as might be expected from a current carried by cations when the extracellular sodium concentration is reduced (see FIG. 6). Moreover, the inwardly and outwardly directed current components were reduced (−7.0±0.8 and 22.7±2.7 pA/pF at −100 and 100 mV, respectively) (see FIG. 6). The use of a hypoosmolar solution (215 mosmol/l) led, after a delay of a few seconds (about 18 seconds on average) to an increase in both the inwardly and outwardly directed currents (FIG. 7). This increase reached its maximum after 50 seconds, on average. The maximum current densities of the inwardly and outwardly directed current were −16.9±1.4 and 66.0±6.1 pA/pF, respectively (n=13). The current-voltage curve of the current activated by hypoosmolar solution had the same shape as the spontaneous current in OTRPC4-expressing cells, although the reverse potential was shifted towards a more positive potential (−5.6±0.7 mV). Removal of sodium and calcium ions from the extracellular solution led to a complete but reversible blocking of the inward current and reduced the outwardly directed current components (see FIG. 7). After the hypotonic solution had been replaced by a solution containing 320 mosmol/l, low current fluxes were measured again, comparable with the starting values before the addition of the hypotonic solution. In the control cells, the addition of hypotonic extracellular solution triggered a current flux which had the properties of chloride channels activated by a change in volume (27). The activation of these currents could be inhibited completely by addition of the chloride channel blocker NPPB (50 μM), whereas the cation currents in OTRPC4-expressing cells triggered by hypotonic solution were unaffected by this blocker.

In order to determine the ion selectivity of OTRPC4, the hypotonic solution used to trigger the currents was replaced by hypotonic solutions containing either sodium on its own or only 20 mM of calcium as cations. The reverse potentials measured were −14.5±8.8 mV (n=5) for a solution containing only 100 mM of sodium and +5.7±1.4 mV (n=5) for a solution containing only 200 mM of calcium. From these values, a ratio of ion permeability of 6.3±0.5 was determined for P_{Ca}/P_{Na} and 0.8±0.3 for P_{Na}/P_{Ca}.

In order to test whether tensile forces on the membrane can trigger the currents carried by OTRPC4, positive and negative pressures were applied to the patch pipette, the whole cell currents being measured both in the cell-attached configuration and in the whole cell configuration. There was no detectable influence on the currents carried by OTRPC4 as a result of pressure changes.

High-Throughput Screen for Identifying a Blocker, Activator or Modulator of the OTRPC4 Cation Channel

The eukaryotic expression plasmid with OTRPC4-cDNA described above additionally contains a gene which confers resistance to the antibiotic G418 on cells transfected with this plasmid. HEK293 cells were transfected by lipofection as described above and stably expressing cells were isolated by selection with G418. In order that the OTRPC4 channel is not constitutively active during the selection period, the HEK293 cells were cultivated in a medium, the osmolarity of which was set to 320 mosmol/l. The HEK293 cells stably expressing the OTRPC4 channel were seeded into 384-well plates and the increase in [Ca²⁺]ᵢ triggered by the addition of hypotonic medium was measured in the cells using a “Fluorescence Imaging Plate Reader” (FLIPR).

Construction of a Recombinant Vector for a Conditional Gene Knock-Out:

For a successful homologous recombination in vivo, an identical sequence 6-8 kb long is needed. The corresponding exon need not be in the centre but an asymmetric arrangement is generally preferred. This allows PCR analysis of the cell clones of the transfected ES cells to be carried out over a short region of about 2-3 kb. A long arm with about 5-6 kb is then left on the other side. Transferred to the existing gene structure of the murine OTRPC4 channel (see FIG. 2) and in an attempt to inactivate the pore region, the following DNA constructs were suitable. Intron 11, situated at base 1965, is greater than 5 kb in size, which means that the DNA for the long arm can be obtained here. Exon 12 with about 420 bp was tagged with flanking LoxP sites for the purpose of a conditional knock-out. Intron 12 with base 2286 bp still offers sufficient size for a short arm. Thus, after the knock-out and the insertion of the neomycin cassette, exon 12 is missing and consequently the channel protein lacks part of the fifth transmembranal region, the pore, the sixth transmembranal region and part of the subsequent cytosolic region. The OTRPC protein which is expressed in the conditional knock-out mice will be functionally inactive in vivo.

Choice of Sequences for Preparing Anti-Sense Oligonucleotides for Inactivating the OTRPC4 Channel:

The anti-sense sequences listed in Table 1 were selected according to the following rules:

The sequences have the least possible homology with the other channels of the OTRPC family.

Clusters of guanines (GGGG) were avoided as they lead to secondary structures and hence non-specific interactions with proteins.

GC and AT base pairs are substantially evenly divided

one of the sequences covers the ATG so as to include, in addition to the induction of an RNAse H which degrades the target RNA, inhibition of the translation start as a mechanism.

TABLE 1

Antisense-Oligo 1/Base 6-21 (5′-UTR)
CGT CTG CAC TGC TCA G (SEQ ID NO:_)
Antisense-Oligo 2/Base 41-55 (coding)
CCT TCG CTG GAA TCC (SEQ ID NO:_)
Antisense-Oligo 3/Base 123-137 (coding)
GAGGAGAGAGGAAAAGC (SEQ ID NO:_)
Antisense-Oligo 4/Base 225-240 (coding)
CAT GCGCAGATTTGGTGC (SEQ ID NO:_)
Antisense-Oligo 5/Base 303-320 (coding)
CACCGAGGACTCATATAG (SEQ ID NO:_)

The anti-sense sequences may also be used as flanking sequences for the construction of ribozymes.

Northern-Blots of RNA from Human Tissues

A probe has been isolated from a human salivary gland cDNA library, based on a partial OTRPC4 clone, and hybridies to several commercial Northern Blots available from Clontech. However, under the conditions employed, no signals were obtained in RNA derived from cardiovascular tissue or tissue of the endocrine or digestive system. On a filter with RNA of 12 human tissues, a signal was obtained in the kidney.

OTRPC4 as a Regulator of the Osmolarity of Body Fluids

Expression of OTRPC4 could be shown in cells of the plexus choroideus by in situ hybridisation. In the region of the plexus, cerebrospinal fluid is formed by secretion processes. It could be shown that hypotonic conditions can trigger calcium influx into isolated porcine Fura.-2 loaded Plexus choroideus primary cells. This shows that OTRPC4 can regullate the osmolarity of body fluids which are formed by secretion, like cerebrospinal fluid, intraocular fluid (and thus intraocular pressure), and saliva. If deviations like hyposmolarity occur, counter-acting processes can be initiated by calcium influx.

Therefore, another aspect of the present invention is the use of modulators, in particular inhibitors or activators of OTRPC4 or its biological activity, for the regulation of osmolarity of body fluids, in particular cerebrospinal fluid, intraocular fluid, and/or saliva, for prophylaxis or treatment of diseases or health conditions where this is necessary. The invention furthermore encompasses pharmaceutical compositions comprising a modulator, activator or inhibitor of OTRPC4, and a pharmaceutically acceptable carrier, diluent, or excipient.

Development of OTRPC4 Specific Antibodies for the Detection of OTRPC4 Proteins

An antibody was generated for the detection of OTRPC4 protein. The antibody is directed to the amino acid sequence CDGHQQGYAPKWRTDDAPL (SEQ ID NO:______), corresponding to the distal C terminus of murine OTRPC4. For generation of the antibody, 1 mg KLH Conjugate of the mentioned petide were injected into rabbits.

The antibody was applied in a Western Blot with three fractions of HEK293 cells transfected with OTRPC4. Native HEK293 cells were used as controls. The fractions from the Cytosol and the cholate extract did not show a signal. In contrast, the cell membrane fraction of the transfected cells showed a clear signal of the presnece of OTRPC4 in the cell membrane (FIG. 8).

HEK293 cells transiently transfected with murine OTRPC4 were tested for the presence of the protein in an immune fluorescence assay. The primary antibody was the antibody described above. The secondary antibody was an anti rabbit antibody conjugated to FITC.

FITC fluoprescence can clearly be seen in the region of the cell membrane, in consistence with the Western Blot data (No figure).

Measurement of OTRPC4 Mediated Change of Intracellualr Calcium Concentration in HEK293 Cells Expressing OTRPC4 Channel

For testing the function of OTRPC4 transfected HEK293 cells with a further method, intracellular Ca²⁺ concentration was measured with FURA-2. The experiments were conducted with luminiscence spectrometer (LS 50 B, Perkin Elmer).

For determining the extent of calcium influx into the cells, maximal intracellular Ca²⁺ was was induced by lonomycin. This [Ca²⁺]ᵢ could be reverted to its original value by EGTA. Influx of extracellular calcium via OTRPC4 was demostrated by showing change of the intracellular Ca²⁺ concentration by lowering osmolarity by 100 mosmol/l (from 320 mosmol/l to 220 mosmol/l). The hypotonic solution led to an increase in [Ca²⁺]ᵢ which could be quenched by EGTA. Half of the [Ca²⁺] increasing effect of hypotonic solution could be blocked by addition of LOE908 (Embaco A, Romanin C, Birke F W, Kukovetz W R, Groschner K: Inhibition of a store-operated Ca2+ entry pathway in human endothelial cells by the isoquinoline derivative LOE 908. British Journal of Pharmacology (1996) 119 702-706) (100 μM) (FIG. 10). The result shows that OTRPC4 is a cation channel which can be blocked by LOE908.

The present invention is not to be limited in scope by the exemplified embodiments which are intended as illustrations of single aspects of the invention. Indeed various modifications of the invention in addition to those shown and described herein will become apparent to those skilled in the art from the foregoing description and accompanying drawings. Such modifications are intended to fall within the scope of the appended claims.

All publications and patent applications cited herein are incorporated by reference in their entireties.

### REFERENCES

1. Lang, F. et al. Functional significance of cell volume regulatory mechanisms. Physiol. Rev. 78, 247-306 (1998).

2. Colbert, H. A. et al. OSM-9, a novel protein with structural similarity to channels, is required for olfaction, mechanosensation and olfactory adaptation in Caenorhabditis elegans. J. Neurosci. 17, 8259-8269 (1997).

3. Montell, C. & Rubin, G. M. Molecular characterization of the Drosophila trp locus: a putative integral membrane protein required for phototransduction. Neuron 2, 1313-1323 (1989).

4. Harteneck, C. et al. From worm to man: three subfamilies of TRP channels. Trends Neurosci. 23, 159-166 (2000).

5. Phillips, A. M. et al. Identification of a Drosophila gene encoding a calmodulin-binding protein with homology to the trp phototransduction gene. Neuron 8, 631-642 (1992).

6. Wes, P. D. et al. TRPC 1, a human homolog of a Drosophila store-operated channel. Proc. Natl. Acad. Sci. USA 92, 9652-9656 (1995).

7. Zhu, X. et al. Molecular cloning of a widely expressed human homologue for the Drosophila trp gene. FEBS Lett. 373, 193-198 (1995).

8. Wissenbach, U. et al. Structure and mRNA expression of a bovine trp homologue related to mammalian trp2. FEBS Lett. 429, 61-66 (1998).

9. Zhu, X. et al. trp, a novel mammalian gene family essential for agonist-activated capacitative Ca²⁺ entry. Cell 85, 661-671 (1996).

10. Philipp, S. et al. A mammalian capacitative calcium entry channel homologous to Drosophila TRP and TRPL. EMBO J. 15, 6166-6171 (1996).

11. Okada, T. et al. Molecular cloning and functional characterization of a novel receptor-activated TRP Ca²⁺ channel from mouse brain. J. Biol. Chem. 273, 10279-10287 (1998).

12. Philipp, S. et al. A novel capacitative calcium entry channel expressed in excitable cells. EMBO J. 17, 4274-4282 (1998).

13. Boulay, G. et al. Cloning and expression of a novel mammalian homologue of Drosophila transient receptor potential (TRP) involved in calcium entry secondary to activation of receptors coupled by the G_{q} class of G protein. J. Biol. Chem. 272, 29672-29680 (1997).

14. Okada, T. et al. Molecular and functional characterization of a novel mouse transient receptor potential protein homologue TRP7. J. Biol. Chem. 274, 27359-27370 (1999).

15. Hofmann, T. et al. Direct activation of human TRPC6 and TRPC3 channels by diacylglycerol. Nature 397, 259-263 (1999).

16. Caterina, M. et al. The capsaicin receptor: a heat-activated ion channel in the pain pathway. Nature 389,816-824(1997).

17. Tominaga, M. et al. The cloned capsaicin receptor integrates multiple pain-producing stimuli. Neuron 21, 531-543 (1998).

18. Caterina, M. et al Capsaicin receptor homologue with a high threshold for noxious heat. Nature 398, 436-441 (1999).

19. Kanzaki, M. et al. Translocation of a calcium-permeable cation channel induced by insulin-like growth factor-I. Nat. Cell Biolog. 1, 165-170 (1999).

20. Hoenderop, J. G. J. et al. Molecular identification of the apical Ca²⁺ channel in 1,25-dihydroxyvitamin D₃-responsive epithelia. J. Biol. Chem. 274, 8375-8378 (1999).

21. Peng, J. B. et al. Molecular cloning and characterization of a channel-like transporter mediating intestinal calcium absorption. J. Biol. Chem. 274, 22739-22746 (1999).

22. Hunter, J. J. et al. Chromosomal localization and genomic characterization of the mouse melastatin gene (Mlsn1). Genomics 54, 116-123 (1998)

23. Nagami, K. et al. Molecular cloning of a novel putative Ca²⁺ channel protein (TRPC7) highly expressed in brain. Genomics 54, 124-131 (1998)

22. Thastrup, O. et al. Thapsigargin, a tumor promoter, discharges intracellular Ca²⁺ strores by specific inhibition of the endoplasmic reticulum Ca²⁺-ATPase. Proc. Natl. Acad. Sci. USA 87, 2466-2470 (1990).

23. Foskett, J. K. in Cellular and Molecular Physiology of Cell Volume Regulation (ed. Strange, K.) 259-277 (CRC Press, Boca Raton, 1994).

24. Yang, X. C. & Sachs, F. Block of stretch-activated ion channels in Xenopus oocytes by gadolinium and calcium ions. Science 243, 1068-1071 (1989).

25. Urbach, V. et al. Mechanosensitive calcium entry and mobilization in renal A6 cells. J. Memb. Biol. 168, 29-37 (1999).

26. Nilius, B. et al. Volume-activated Cl-channels. Gen. Pharmacol. 27, 1131-1140 (1996).

What is claimed is:

1. Nucleic acid, characterised in that it codes for the non-selective cation channel OTRPC4 or for a fragment, a functional variant, an allelic variant or a subunit, or variants of said nucleic acid on the basis of the degenerative code or a nucleic acid which is able to hybridise with said nucleic acid.

2. Nucleic acid according to claim 1, characterised in that it is RNA.

3. Nucleic acid according to claim 1, characterised in that it is DNA.

4. Nucleic acid according to one of claim 1 or 3, characterised in that it contains 5′ or 3′ or 5′ and 3′ untranslated regions.

5. Nucleic acid according to one of claims 1 to 4, characterised in that it codes for a fragment of the non-selective cation channel OTRPC4.

6 Nucleic acid according to one of claims 1 to 5, characterised in that it codes for a functional variant of the non-selective cation channel OTRPC4.

7. Nucleic acid according to one of claims 1 to 6, characterised in that it codes for an allelic variant of the non-selective cation channel OTRPC4.

8. Nucleic acid according to one of claims 1 to 7, characterised in that it codes for variants of nucleic acid on the basis of the degenerative code.

9. Nucleic acid, characterised in that it is capable of hybridising with a nucleic acid according to one of claims 1 to 8 under stringent conditions.

10. Nucleic acid according to one of claims 1 to 9, characterised in that the said non-selective cation channel OTRPC4 is a mammalian cation channel.

11. Nucleic acid according to one of claims 1 to 10, characterised in that the said non-selective cation channel OTRPC4 is murine.

12. Nucleic acid according to one of claims 1 to 11, characterised in that the said non-selective cation channel OTRPC4 is human.

13. Nucleic acid, characterised in that it contains the sequence
CTCTCACCGCCTACTACCAGCCGCTGGAGGGCACAATGGCGGATTCCAGC
GAAGGCCCCCGCGCGGGGCCCGGGGAGGTGGCTGAGCTCCCCGGGGATGA
GAGTGGCACCCCAGGTGGGGAGGCTTTTCCTCTCTCCTCCCTGGCCAATC
TGTTTGAGGGGGAGGATGGCTCCCTTTCGCCCTCACCGGCTGATGCCAGT
CGCCCTGCTGGCCCAGGCGATGGGCGACCAAATCTGCGCATGAAGTTCCA
GGGCGCCTTCCGCAAGGGGGTGCCCAACCCCATCGATCTGCTGGAGTCCA
CCCTATATGAGTCCTCGGTGGTGCGTGGGCCCAAGAAAGCACCCATGGAC
TCACTGTTTGACTACGGCACCTATCGTCACCACTCCAGTGACAACAAGAG
GTGGAGGAAGAAGATCATAGAGAAGCAGCCGCAGAGCCCCAAAGCCCCTG
CCCCTCAGCCGCCCCCCATCCTCAAAGTCTTCAACCGGCCTATCCTCTTT
GACATCGTGTCCCGGGGCTCCACTGCTGACCTGGACGGGCTGCTCCCATT
CTTGCTGACCCACAAGAAACGCCTAACTGATGAGGAGTTTCGAGAGCCAT
CTACGGGGAAGACCTGCCTGCCCAAGGCCTTGCTGAACCTGAGCAATGGC
CGCAACGACACCATCCCTGTGCTGCTGGACATCGCGGAGCGCACCGGCAA
CATGCGGGAGTTCATTAACTCGCCCTTCCGTGACATCTACTATCGAGGTC
AGACAGCCCTGCACATCGCCATTGAGCGTCGCTGCAAACACTACGTGGAA
CTTCTCGTGGCCCAGGGAGCTGATGTCCACGCCCAGGCCCGTGGGCGCTT
CTTCCAGCCCAAGGATGAGGGGGGCTACTTCTACTTTGGGGAGCTGCCCC
TGTCGCTGGCTGCCTGCACCAACCAGCCCCACATTGTCAACTACCTGACG
GAGAACCCCCACAAGAAGGCGGACATGCGGCGCCAGGACTCGCGAGGCAA
CACAGTGCTGCATGCGCTGGTGGCCATTGCTGACAACACCCGTGAGAACA
CCAAGTTTGTTACCAAGATGTACGACCTGCTGCTGCTCAAGTGTGCCCGC
CTCTTCCCCGACAGCAACCTGGAGGCCGTGCTCAACAACGACGGCCTCTC
GCCCCTCATGATGGCTGCCAAGACGGGCAAGATTGGGATCTTTCAGCACA
TCATCCGGCGGGAGGTGACGGATGAGGACACACGGCACCTGTCCCGCAAG
TTCAAGGACTGGGCCTATGGGCCAGTGTATTCCTCGCTTTATGACCTCTC
CTCCCTGGACACGTGTGGGGAAGAGGCCTCCGTGCTGGAGATCCTGGTGT
ACAACAGCAAGATTGAGAACCGCCACGAGATGCTGGCTGTGGAGCCCATC
AATGAACTGCTGCGGGACAAGTGGCGCAAGTTCGGGGCCGTCTCCTTCTA
CATCAACGTGGTCTCCTACCTGTGTGCCATGGTCATCTTCACTCTCACCG
CCTACTACCAGCCGCTGGAGGGCACACCGCCGTACCCTTACCGCACCACG
GTGGACTACCTGCGGCTGGCTGGCGAGGTCATTACGCTCTTCACTGGGGT
CCTGTTCTTCTTCACCAACATCAAAGACTTGTTCATGAAGAAATGCCCTG
GAGTGAATTCTCTCTTCATTGATGGCTCCTTCCAGCTGCTCTACTTCATC
TACTCTGTCCTGGTGATCGTCTCAGCAGCCCTCTACCTGGCAGGGATCGA
GGCCTACCTGGCCGTGATGGTCTTTGCCCTGGTCCTGGGCTGGATGAATG
CCCTTTACTTCACCCGTGGGCTGAAGCTGACGGGGACCTATAGCATCATG
ATCCAGAAGATTCTCTTCAAGGACCTTTTCCGATTCCTGCTCGTCTACTT
GCTCTTCATGATCGGCTACGCTTCAGCCCTGGTCTCCCTCCTGAACCCGT
GTGCCAACATGAAGGTGTGCAATGAGGACCAGACCAACTGCACAGTGCCC
ACTTACCCCTCGTGCCGTGACAGCGAGACCTTCAGCACCTTCCTCCTGGA
CCTGTTTAAGCTGACCATCGGCATGGGCGACCTGGAGATGCTGAGCAGCA
CCAAGTACCCCGTGGTCTTCATCATCCTGCTGGTGACCTACATCATCCTC
ACCTTTGTGCTGCTCCTCAACATGCTCATTGCCCTCATGGGCGAGACAGT
GGGCCAGGTCTCCAAGGAGAGCAAGCACATCTGGAAGCTGCAGTGGGCCA
CCACCATCCTGGACATTGAGCGCTCCTTCCCCGTATTCCTGAGGAAGGCC
TTCCGCTCTGGGGAGATGGTCACCGTGGGCAAGAGCTCGGACGGCACTCC
TGACCGCAGGTGGTGCTTCAGGGTGGATGAGGTGAACTGGTCTCACTGGA
ACCAGAACTTGGGCATCATCAACGAGGACCCGGGCAAGAATGAGACCTAC
CAGTATTATGGCTTCTCGCATACCGTGGGCCGCCTCCGCAGGGATCGCTG
GTCCTCGGTGGTACCCCGCGTGGTGGAACTGAACAAGAACTCGAACCCGG
ACGAGGTGGTGGTGCCTCTGGACAGCATGGGGAACCCCCGCTGCGATGGC
CACCAGCAGGGTTACCCCCGCAAGTGGAGGACTGAGGACGCCCCGCTCTA
GGGACTGCAGCCCAGCCCCAGCTTCTCTGCCCACTCATTTCTAGTCCAGC
CGCATTTCAGCAGTGCCTTCTGGGGTGTCCCCCCACACCCTGCTTTGGCC
CCAGAGGCGAGGGACCAGTGGAGGTGCCAGGGAGGCCCCAGGACCCTGTG
GTCCCCTGGCTCTGCCTCCCCACCCTGGGGTGGGGGCTCCCGGCCACCTG
TCTTGCTCCTATGGAGTCACATAAGCCAACGCCAGAGCCCCTCCACCTCA
GGCCCCAGCCCCTGCCTCTCCATTATTTATTTGCTCTGCTCTCAGGAAGC
GACGTGACCCCTGCCCCAGCTGGAACCTGGCAGAGGCCTTAGGACCCCGT
TCCAAGTGCACTGCCCGGCCAAGCCCCAGCCTCAGCCTGCGCCTGAGCTG
CATGCGCCACCATTTTTGGCAGCGTGGCAGCTTTGCAAGGGGCTGGGGCC
CTCGGCGTGGGGCCATGCCTTCTGTGTGTTCTGTAGTGTCTGGGATTTGC
CGGTGCTCAATAAATGTTTATTCATTGACGGTGAAAAAAAAAAAAAAAAA
AA
or a partial sequence thereof, a nucleic acid which is capable of hybridising with said sequence under stringent conditions, an allelic variant or a functional variant of said sequence or a variant of nucleic acid on the basis of the degenerative code.

14. Nucleic acid, characterised in that it has the sequence
CTCTCACCGCCTACTACCAGCCGCTGGAGGGCACAATGGCGGATTCCAGC
GAAGGCCCCCGCGCGGGGCCCGGGGAGGTGGCTGAGCTCCCCGGGGATGA
GAGTGGCACCCCAGGTGGGGAGGCTTTTCCTCTCTCCTCCCTGGCCAATC
TGTTTGAGGGGGAGGATGGCTCCCTTTCGCCCTCACCGGCTGATGCCAGT
CGCCCTGCTGGCCCAGGCGATGGGCGACCAAATCTGCGCATGAAGTTCCA
GGGCGCCTTCCGCAAGGGGGTGCCCAACCCCATCGATCTGCTGGAGTCCA
CCCTATATGAGTCCTCGGTGGTGCCTGGGCCCAAGAAAGCACCCATGGAC
TCACTGTTTGACTACGGCACCTATCGTCACCACTCCAGTGACAACAAGAG
GTGGAGGAAGAAGATCATAGAGAAGCAGCCGCAGAGCCCCAAAGCCCCTG
CCCCTCAGCCGCCCCCCATCCTCAAAGTCTTCAACCGGCCTATCCTCTTT
GACATCGTGTCCCGGGGCTCCACTGCTGACCTGGACGGGCTGCTCCCATT
CTTGCTGACCCACAAGAAACGCCTAACTGATGAGGAGTTTCGAGAGCCAT
CTACGGGGAAGACCTGCCTGCCCAAGGCCTTGCTGAACCTGAGCAATGGC
CGCAACGACACCATCCCTGTGCTGCTGGACATCGCGGAGCGCACCGGCAA
CATGCGGGAGTTCATTAACTCGCCCTTCCGTGACATCTACTATCGAGGTC
AGACAGCCCTGCACATCGCCATTGAGCGTCGCTGCAAACACTACGTGGAA
CTTCTCGTGGCCCAGGGAGCTGATGTCCACGCCCAGGCCCGTGGGCGCTT
CTTCCAGCCCAAGGATGAGGGGGGCTACTTCTACTTTGGGGAGCTGCCCC
TGTCGCTGGCTGCCTGCACCAACCAGCCCCACATTGTCAACTACCTGACG
GAGAACCCCCACAAGAAGGCGGACATGCGGCGCCAGGACTCGCGAGGCAA
CACAGTGCTGCATGCGCTGGTGGCCATTGCTGACAACACCCGTGAGAACA
CCAAGTTTGTTACCAAGATGTACGACCTGCTGCTGCTCAAGTGTGCCCGC
CTCTTCCCCGACAGCAACCTGGAGGCCGTGCTCAACAACGACGGCCTCTC
GCCCCTCATGATGGCTGCCAAGACGGGCAAGATTGGGATCTTTCAGCACA
TCATCCGGCGGGAGGTGACGGATGAGGACACACGGCACCTGTCCCGCAAG
TTCAAGGACTGGGCCTATGGGCCAGTGTATTCCTCGCTTTATGACCTCTC
CTCCCTGGACACGTGTGGGGAAGAGGCCTCCGTGCTGGAGATCCTGGTGT
ACAACAGCAAGATTGAGAACCGCCACGAGATGCTGGCTGTGGAGCCCATC
AATGAACTGCTGCGGGACAAGTGGCGCAAGTTCGGGGCCGTCTCCTTCTA
CATCAACGTGGTCTCCTACCTGTGTGCCATGGTCATCTTCACTCTCACCG
CCTACTACCAGCCGCTGGAGGGCACACCGCCGTACCCTTACCGCACCACG
GTGGACTACCTGCGGCTGGCTGGCGAGGTCATTACGCTCTTCACTGGGGT
CCTGTTCTTCTTCACCAACATCAAAGACTTGTTCATGAAGAAATGCCCTG
GAGTGAATTCTCTCTTCATTGATGGCTCCTTCCAGCTGCTCTACTTCATC
TACTCTGTCCTGGTGATCGTCTCAGCAGCCCTCTACCTGGCAGGGATCGA
GGCCTACCTGGCCGTGATGGTCTTTGCCCTGGTCCTGGGCTGGATGAATG
CCCTTTACTTCACCCGTGGGCTGAAGCTGACGGGGACCTATAGCATCATG
ATCCAGAAGATTCTCTTCAAGGACCTTTTCCGATTCCTGCTCGTCTACTT
GCTCTTCATGATCGGCTACGCTTCAGCCCTGGTCTCCCTCCTGAACCCGT
GTGCCAACATGAAGGTGTGCAATGAGGACCAGACCAACTGCACAGTGCCC
ACTTACCCCTCGTGCCGTGACAGCGAGACCTTCAGCACCTTCCTCCTGGA
CCTGTTTAAGCTGACCATCGGCATGGGCGACCTGGAGATGCTGAGCAGCA
CCAAGTACCCCGTGGTCTTCATCATCCTGCTGGTGACCTACATCATCCTC
ACCTTTGTGCTGCTCCTCAACATGCTCATTGCCCTCATGGGCGAGACAGT
GGGCCAGGTCTCCAAGGAGAGCAAGCACATCTGGAAGCTGCAGTGGGCCA
CCACCATCCTGGACATTGAGCGCTCCTTCCCCGTATTCCTGAGGAAGGCC
TTCCGCTCTGGGGAGATGGTCACCGTGGGCAAGAGCTCGGACGGCACTCC
TGACCGCAGGTGGTGCTTCAGGGTGGATGAGGTGAACTGGTCTCACTGGA
ACCAGAACTTGGGCATCATCAACGAGGACCCGGGCAAGAATGAGACCTAC
CAGTATTATGGCTTCTCGCATACCGTGGGCCGCCTCCGCAGGGATCGCTG
GTCCTCGGTGGTACCCCGCGTGGTGGAACTGAACAAGAACTCGAACCCGG
ACGAGGTGGTGGTGCCTCTGGACAGCATGGGGAACCCCCGCTGCGATGGC
CACCAGCAGGGTTACCCCCGCAAGTGGAGGACTGAGGACGCCCCGCTCTA
GGGACTGCAGCCCAGCCCCAGCTTCTCTGCCCACTCATTTCTAGTCCAGC
CGCATTTCAGCAGTGCCTTCTGGGGTGTCCCCCCACACCCTGCTTTGGCC
CCAGAGGCGAGGGACCAGTGGAGGTGCCAGGGAGGCCCCAGGACCCTGTG
GTCCCCTGGCTCTGCCTCCCCACCCTGGGGTGGGGGCTCCCGGCCACCTG
TCTTGCTCCTATGGAGTCACATAAGCCAACGCCAGAGCCCCTCCACCTCA
GGCCCCAGCCCCTGCCTCTCCATTATTTATTTGCTCTGCTCTCAGGAAGC
GACGTGACCCCTGCCCCAGCTGGAACCTGGCAGAGGCCTTAGGACCCCGT
TCCAAGTGCACTGCCCGGCCAAGCCCCAGCCTCAGCCTGCGCCTGAGCTG
CATGCGCCACCATTTTTGGCAGCGTGGCAGCTTTGCAAGGGGCTGGGGCC
CTCGGCGTGGGGCCATGCCTTCTGTGTGTTCTGTAGTGTCTGGGATTTGC
CGGTGCTCAATAAATGTTTATTCATTGACGGTGAAAAAAAAAAAAAAAAA
AA

15. Nucleic acid, characterised in that it contains the sequence
ATGGCGGATTCCAGCGAAGGCCCCCGCGCGGGGCCCGGGGAGGTGGCTGA
GCTCCCCGGGGATGAGAGTGGCACCCCAGGTGGGGAGGCTTTTCCTCTCT
CCTCCCTGGCCAATCTGTTTGAGGGGGAGGATGGCTCCCTTTCGCCCTCA
CCGGCTGATGCCAGTCGCCCTGCTGGCCCAGGCGATGGGCGACCAAATCT
GCGCATGAAGTTCCAGGGCGCCTTCCGCAAGGGGGTGCCCAACCCCATCG
ATCTGCTGGAGTCCACCCTATATGAGTCCTCGGTGGTGCCTGGGCCCAAG
AAAGCACCCATGGACTCACTGTTTGACTACGGCACCTATCGTCACCACTC
CAGTGACAACAAGAGGTGGAGGAAGAAGATCATAGAGAAGCAGCCGCAGA
GCCCCAAAGCCCCTGCCCCTCAGCCGCCCCCCATCCTCAAAGTCTTCAAC
CGGCCTATCCTCTTTGACATCGTGTCCCGGGGCTCCACTGCTGACCTGGA
CGGGCTGCTCCCATTCTTGCTGACCCACAAGAAACGCCTAACTGATGAGG
AGTTTCGAGAGCCATCTACGGGGAAGACCTGCCTGCCCAAGGCCTTGCTG
AACCTGAGCAATGGCCGCAACGACACCATCCCTGTGCTGCTGGACATCGC
GGAGCGCACCGGCAACATGCGGGAGTTCATTAACTCGCCCTTCCGTGACA
TCTACTATCGAGGTCAGACAGCCCTGCACATCGCCATTGAGCGTCGCTGC
AAACACTACGTGGAACTTCTCGTGGCCCAGGGAGCTGATGTCCACGCCCA
GGCCCGTGGGCGCTTCTTCCAGCCCAAGGATGAGGGGGGCTACTTCTACT
TTGGGGAGCTGCCCCTGTCGCTGGCTGCCTGCACCAACCAGCCCCACATT
GTCAACTACCTGACGGAGAACCCCCACAAGAAGGCGGACATGCGGCGCCA
GGACTCGCGAGGCAACACAGTGCTGCATGCGCTGGTGGCCATTGCTGACA
ACACCCGTGAGAACACCAAGTTTGTTACCAAGATGTACGACCTGCTGCTG
CTCAAGTGTGCCCGCCTCTTCCCCGACAGCAACCTGGAGGCCGTGCTCAA
CAACGACGGCCTCTCGCCCCTCATGATGGCTGCCAAGACGGGCAAGATTG
GGATCTTTCAGCACATCATCCGGCGGGAGGTGACGGATGAGGACACACGG
CACCTGTCCCGCAAGTTCAAGGACTGGGCCTATGGGCCAGTGTATTCCTC
GCTTTATGACCTCTCCTCCCTGGACACGTGTGGGGAAGAGGCCTCCGTGC
TGGAGATCCTGGTGTACAACAGCAAGATTGAGAACCGCCACGAGATGCTG
GCTGTGGAGCCCATCAATGAACTGCTGCGGGACAAGTGGCGCAAGTTCGG
GGCCGTCTCCTTCTACATCAACGTGGTCTCCTACCTGTGTGCCATGGTCA
TCTTCACTCTCACCGCCTACTACCAGCCGCTGGAGGGCACACCGCCGTAC
CCTTACCGCACCACGGTGGACTACCTGCGGCTGGCTGGCGAGGTCATTAC
GCTCTTCACTGGGGTCCTGTTCTTCTTCACCAACATCAAAGACTTGTTCA
TGAAGAAATGCCCTGGAGTGAATTCTCTCTTCATTGATGGCTCCTTCCAG
CTGCTCTACTTCATCTACTCTGTCCTGGTGATCGTCTCAGCAGCCCTCTA
CCTGGCAGGGATCGAGGCCTACCTGGCCGTGATGGTCTTTGCCCTGGTCC
TGGGCTGGATGAATGCCCTTTACTTCACCCGTGGGCTGAAGCTGACGGGG
ACCTATAGCATCATGATCCAGAAGATTCTCTTCAAGGACCTTTTCCGATT
CCTGCTCGTCTACTTGCTCTTCATGATCGGCTACGCTTCAGCCCTGGTCT
CCCTCCTGAACCCGTGTGCCAACATGAAGGTGTGCAATGAGGACCAGACC
AACTGCACAGTGCCCACTTACCCCTCGTGCCGTGACAGCGAGACCTTCAG
CACCTTCCTCCTGGACCTGTTTAAGCTGACCATCGGCATGGGCGACCTGG
AGATGCTGAGCAGCACCAAGTACCCCGTGGTCTTCATCATCCTGCTGGTG
ACCTACATCATCCTCACCTTTGTGCTGCTCCTCAACATGCTCATTGCCCT
CATGGGCGAGACAGTGGGCCAGGTCTCCAAGGAGAGCAAGCACATCTGGA
AGCTGCAGTGGGCCACCACCATCCTGGACATTGAGCGCTCCTTCCCCGTA
TTCCTGAGGAAGGCCTTCCGCTCTGGGGAGATGGTCACCGTGGGCAAGAG
CTCGGACGGCACTCCTGACCGCAGGTGGTGCTTCAGGGTGGATGAGGTGA
ACTGGTCTCACTGGAACCAGAACTTGGGCATCATCAACGAGGACCCGGGC
AAGAATGAGACCTACCAGTATTATGGCTTCTCGCATACCGTGGGCCGCCT
CCGCAGGGATCGCTGGTCCTCGGTGGTACCCCGCGTGGTGGAACTGAACA
AGAACTCGAACCCGGACGAGGTGGTGGTGCCTCTGGACAGCATGGGGAAC
CCCCGCTGCGATGGCCACCAGCAGGGTTACCCCCGCAAGTGGAGGACTGA
GGACGCCCCGCTCTAG
or a partial sequence thereof, a nucleic acid which is capable of hybridising with said sequence under stringent conditions, an allelic variant or a functional variant of said sequence or a variant of nucleic acid on the basis of the degenerative code.

16. Nucleic acid, characterised in that it has the sequence
ATGGCGGATTCCAGCGAAGGCCCCCGCGCGGGGCCCGGGGAGGTGGCTGA
GCTCCCCGGGGATGAGAGTGGCACCCCAGGTGGGGAGGCTTTTCCTCTCT
CCTCCCTGGCCAATCTGTTTGAGGGGGAGGATGGCTCCCTTTCGCCCTCA
CCGGCTGATGCCAGTCGCCCTGCTGGCCCAGGCGATGGGCGACCAAATCT
GCGCATGAAGTTCCAGGGCGCCTTCCGCAAGGGGGTGCCCAACCCCATCG
ATCTGCTGGAGTCCACCCTATATGAGTCCTCGGTGGTGCCTGGGCCCAAG
AAAGCACCCATGGACTCACTGTTTGACTACGGCACCTATCGTCACCACTC
CAGTGACAACAAGAGGTGGAGGAAGAAGATCATAGAGAAGCAGCCGCAGA
GCCCCAAAGCCCCTGCCCCTCAGCCGCCCCCCATCCTCAAAGTCTTCAAC
CGGCCTATCCTCTTTGACATCGTGTCCCGGGGCTCCACTGCTGACCTGGA
CGGGCTGCTCCCATTCTTGCTGACCCACAAGAAACGCCTAACTGATGAGG
AGTTTCGAGAGCCATCTACGGGGAAGACCTGCCTGCCCAAGGCCTTGCTG
AACCTGAGCAATGGCCGCAACGACACCATCCCTGTGCTGCTGGACATCGC
GGAGCGCACCGGCAACATGCGGGAGTTCATTAACTCGCCCTTCCGTGACA
TCTACTATCGAGGTCAGACAGCCCTGCACATCGCCATTGAGCGTCGCTGC
AAACACTACGTGGAACTTCTCGTGGCCCAGGGAGCTGATGTCCACGCCCA
GGCCCGTGGGCGCTTCTTCCAGCCCAAGGATGAGGGGGGCTACTTCTACT
TTGGGGAGCTGCCCCTGTCGCTGGCTGCCTGCACCAACCAGCCCCACATT
GTCAACTACCTGACGGAGAACCCCCACAAGAAGGCGGACATGCGGCGCCA
GGACTCGCGAGGCAACACAGTGCTGCATGCGCTGGTGGCCATTGCTGACA
ACACCCGTGAGAACACCAAGTTTGTTACCAAGATGTACGACCTGCTGCTG
CTCAAGTGTGCCCGCCTCTTCCCCGACAGCAACCTGGAGGCCGTGCTCAA
CAACGACGGCCTCTCGCCCCTCATGATGGCTGCCAAGACGGGCAAGATTG
GGATCTTTCAGCACATCATCCGGCGGGAGGTGACGGATGAGGACACACGG
CACCTGTCCCGCAAGTTCAAGGACTGGGCCTATGGGCCAGTGTATTCCTC
GCTTTATGACCTCTCCTCCCTGGACACGTGTGGGGAAGAGGCCTCCGTGC
TGGAGATCCTGGTGTACAACAGCAAGATTGAGAACCGCCACGAGATGCTG
GCTGTGGAGCCCATCAATGAACTGCTGCGGGACAAGTGGCGCAAGTTCGG
GGCCGTCTCCTTCTACATCAACGTGGTCTCCTACCTGTGTGCCATGGTCA
TCTTCACTCTCACCGCCTACTACCAGCCGCTGGAGGGCACACCGCCGTAC
CCTTACCGCACCACGGTGGACTACCTGCGGCTGGCTGGCGAGGTCATTAC
GCTCTTCACTGGGGTCCTGTTCTTCTTCACCAACATCAAAGACTTGTTCA
TGAAGAAATGCCCTGGAGTGAATTCTCTCTTCATTGATGGCTCCTTCCAG
CTGCTCTACTTCATCTACTCTGTCCTGGTGATCGTCTCAGCAGCCCTCTA
CCTGGCAGGGATCGAGGCCTACCTGGCCGTGATGGTCTTTGCCCTGGTCC
TGGGCTGGATGAATGCCCTTTACTTCACCCGTGGGCTGAAGCTGACGGGG
ACCTATAGCATCATGATCCAGAAGATTCTCTTCAAGGACCTTTTCCGATT
CCTGCTCGTCTACTTGCTCTTCATGATCGGCTACGCTTCAGCCCTGGTCT
CCCTCCTGAACCCGTGTGCCAACATGAAGGTGTGCAATGAGGACCAGACC
AACTGCACAGTGCCCACTTACCCCTCGTGCCGTGACAGCGAGACCTTCAG
CACCTTCCTCCTGGACCTGTTTAAGCTGAGCATCGGCATGGGCGACCTGG
AGATGCTGAGCAGCACCAAGTACCCCGTGGTCTTCATCATCCTGCTGGTG
ACCTACATCATCCTCACCTTTGTGCTGCTCCTCAACATGCTCATTGCCCT
CATGGGCGAGACAGTGGGCCAGGTCTCCAAGGAGAGCAAGCACATCTGGA
AGCTGCAGTGGGCCACCACCATCCTGGACATTGAGCGCTCCTTCCCCGTA
TTCCTGAGGAAGGCCTTCCGCTCTGGGGAGATGGTCACCGTGGGCAAGAG
CTCGGACGGCACTCCTGACCGCAGGTGGTGCTTCAGGGTGGATGAGGTGA
ACTGGTCTCACTGGAACCAGAACTTGGGCATCATCAACGAGGACCCGGGC
AAGAATGAGACCTACCAGTATTATGGCTTCTCGCATACCGTGGGCCGCCT
CCGCAGGGATCGCTGGTCCTCGGTGGTACCCCGCGTGGTGGAACTGAACA
AGAACTCGAACCCGGACGAGGTGGTGGTGCCTCTGGACAGCATGGGGAAC
CCCCGCTGCGATGGCCACCAGCAGGGTTACCCCCGCAAGTGGAGGACTGA
GGACGCCCCGCTCTAG.

17. Nucleic acid, characterised in that it comprises the sequence
GGCCACGCGTCGACTAGTACGGGGGGGGGGGGGGGGGTGGCRGSRGGAKC
AGGACTCGGCCGGAGGGATCAGGAAGCGGCGGCGCTGCGCCCGCGTCCTG
AGGCTGAGAAGTACAAACAGATCTGGGTCCAGTATGGCAGATCCTGGTGA
TGGTCCCCGTGCAGCGCCTGGGGAGGTGGCTGAGCCCCCTGGAGATGAGA
GTGGTACCTCTGGTGGGGAGGCCTTCCCCCTCTCTTCCGTGGCCAATCTG
TTTGAGGGGGAGGAAGGCTCCTCTTCTCTTTCCCCGGTGGATGCTAGCCG
CCCTGCTGGCCCTGGCGATGGACGTCCAAACCTGCGTATGAAGTTCCAGG
GCGCTTTCCGCAAGGGGGTTCCCAACCCCATTGACCTGTTGGAGTCCACC
CGGTACGAGTCCTCAGTAGTGCCTGGGCCCAAGAAAGCGCCCATGGATTC
CTTGTTCGACTACGGCACTTACCGTCACCACCCCAGTGACAACAAGAGAT
GGAGGAGAAAGGTCGTGGAGAAGCAGCCACAGAGCCCCAAAGCTCCTGCA
CGCCAGCCACCCCCCATCCTCAAAGTCTTCAATCGGCCCATCCTCTTTGA
CATTGTGTCCCGGGGCTCCACTGCGGACCTAGATGGACTGCTCTCCTTCT
TGTTGACCCACAAGAAGCGCCTGACTGATGAGGAGTTCCGGGAGCCGTCC
ACGGGGAAGACCTGCCTGCCCAAGGCGCTGCTGAACCTAAGCAACGGGCG
CAACGACACCATCCCGGTGTTGCTGGACATTGCGGAGCGCACCGGCAACA
TGCGTGAATTCATCAACTCGCCCTTCAGAGACATCTACTACCGAGGCCAG
ACATCCCTGCACATTGCCATCGAACGGCGCTGCAAGCACTACGTGGAGCT
GCTGGTGGCCCAGGGAGCCGACGTGCACGCCCAGGCCCGCGGCCGCTTCT
TCCAGCCCAAGGATGAGGGAGGCTACTTCTACTTTGGGGAGCTGCCCTTG
TCCCTGGCAGCCTGCACCAACCAGCCGCACATCGTCAACTACCTGACAGA
GAACCCTCACAAGAAAGCTGACATGAGGCGACAGGACTCGAGGGGGAACA
CGGTGCTGCACGCGCTGGTGGCCATCGCCGACAACACCCGAGAGAACACC
AAGTTTGTCACCAAGATGTACGACCTGCTGCTTCTCAAGTGTTCACGCCT
CTTCCCCGACAGCAACCTGGAGACAGTTCTCAACAATGATGGCCTTTCGC
CTCTCATGATGGCTGCCAAGACAGGCAAGATCGGGGTCTTTCAGCACATC
ATCCGACGTGAGGTGACAGATGAGGACACCCGGCATCTGTCTCGCAAGTT
CAAGGACTGGGCCTATGGGCCTGTGTATTCTTCTCTCTACGACCTCTCCT
CCCTGGACACATGCGGGGAGGAGGTGTCCGTGCTGGAGATCCTGGTGTAC
AACAGCAAGATCGAGAACCGCCATGAGATGCTGGCTGTAGAGCCCATTAA
CGAACTGTTGAGAGACAAGTGGCGTAAGTTTGGGGCTGTGTCCTTCTACA
TCAACGTGGTCTCCTATCTGTGTGCCATGGTCATCTTCACCCTCACCGCC
TACTATCAGCCACTGGAGGGCACGCCACCCTACCCTTACCGGACCACAGT
GGACTACCTGAGGCTGGCTGGCGAGGTCATCACGCTCTTCACAGGAGTCC
TGTTCTTCTTTACCAGTATCAAAGACTTGTTCACGAAGAAATGCCCTGGA
GTGAATTCTCTCTTCGTCGATGGCTCCTTCCAGTTACTCTACTTCATCTA
CTCTGTGCTGGTGGTTGTCTCTGCGGCGCTCTACCTGGCTGGGATCGAGG
CCTACCTGGCTGTGATGGTCTTTGCCCTGGTCCTGGGCTGGATGAATGCG
CTGTACTTCACGCGCGGGTTGAAGCTGACGGGGACCTACAGCATCATGAT
TCAGAAGATCCTCTTCAAAGACCTCTTCCGCTTCCTGCTTGTGTACCTGC
TCTTCATGATCGGCTATGCCTCAGCCCTGGTCACCCTCCTGAATCCGTGC
ACCAACATGAAGGTCTGTGACGAGGACCAGAGCAACTGCACGGTGCCCAC
GTATCCTGCGTGCCGCGACAGCGAGACCTTCAGCGCCTTCCTCCTGGACC
TCTTCAAGCTCACCATCGGCATGGGAGACCTGGAGATGCTGAGCAGCGCC
AAGTACCCCGTGGTCTTCATCCTCCTGCTGGTCACCTACATCATCCTCAC
CTTCGTGCTCCTGTTGAACATGCTTATCGCCCTCATGGGTGAGACCGTGG
GCCAGGTGTCCAAGGAGAGCAAGCACATCTGGAAGTTGCAGTGGGCCACC
ACCATCCTGGACATCGAGCGTTCCTTCCCTGTGTTCCTGAGGAAGGCCTT
CCGCTCCGGAGAGATGGTGACTGTGGGCAAGAGCTCAGATGGCACTCCGG
ACCGCAGGTGGTGCTTCAGGGTGGACGAGGTGAACTGGTCTCACTGGAAC
CAGAACTTGGGCATCATTAACGAGGACCCTGGCAAGAGTGAAATCTACCA
GTACTATGGCTTCTCCCACACCGTGGGGCGCCTTCGTAGGGATCGTTGGT
CCTCGGTGGTGCCCCGCGTAGTGGAGCTGAACAAGAACTCAAGCGCAGAT
GAAGTGGTGGTACCCCTGGATAACCTAGGGAACCCCAACTGTGACGGCCA
CCAGCAGGGCTACGCTCCCAAGTGGAGGACGGACGATGCCCCACTGTAGG
GGCCGTGCCAGAGCTCGCACAGATAGTCCAGGCTTGGCCTTCGCTCCCAC
CTACATTTAGGCATTTGTCCGGTGTCTTCCCACACCCGCATGGGACCTTG
GAGGTGAGGGCCTCTGTGGCGACTCTGTGGAGGCCCCAGGACCCTCTGGT
CCCCGCCAAGACTTTTGCCTTCAGCTCTACTCCCCACATGGGGGGGCGGG
GCTCCTGGCTACCTGTCTCGCTCGCTCCCATGGAGTCACCTAAGCCAGCA
CAAGGCCCCTCTCCTCGAAAGGCTCAGGCCCCATCCCTCTTGTGTATTAT
TTATTGCTCTCCTCAGGAAAATGGGGTGGCAGGAGTCCACCCGCGGCTGG
AACCTGGCCAGGGCTGAAGCTCATGCAGGGACGCTGCAGCTCCGACCTGC
CACAGATCTGACCTGCTGCAGCCCTGGCTAGTGTGGGTCTTCTGTACTTT
GAAGAGATCGGGGCCGCTGGTGCTCAATAAATGTTTATTCTCGGTGGAAA
AAAAAAAAAAAAAAAAAAAAAAAAAAAAAAAAAAAAAAAAAAAAAAAAAA
AAAAAAAAAAAAAAAAAAAAAAAA
or a partial sequence thereof, a nucleic acid which is capable of hybridising with said sequence under stringent conditions, an allelic variant or a functional variant of said sequence or a variant of the nucleic acid on the basis of the degenerative code, wherein R may be an A or G, M may be an A or C, S may be a C or G, Y may be a C or T, K may be a G or T and W may be an A or T.

18. Nucleic acid, characterised in that it has the sequence
GGCCACGCGTCGACTAGTACGGGGGGGGGGGGGGGGGTGGCRGSRGGAKC
AGGACTCGGCCGGAGGGATCAGGAAGCGGCGGCGCTGCGCCCGCGTCCTG
AGGCTGAGAAGTACAAACAGATCTGGGTCCAGTATGGCAGATCCTGGTGA
TGGTCCCCGTGCAGCGCCTGGGGAGGTGGCTGAGCCCCCTGGAGATGAGA
GTGGTACCTCTGGTGGGGAGGCCTTCCCCCTCTCTTCCCTGGCCAATCTG
TTTGAGGGGGAGGAAGGCTCCTCTTCTCTTTCCCCGGTGGATGCTAGCCG
CCCTGCTGGCCCTGGCGATGGACGTCCAAACCTGCGTATGAAGTTCcAGG
GCGCTTTCCGCAAGGGGGTTCCCAACCCCATTGACCTGTTGGAGTCGACC
CGGTACGAGTCCTCAGTAGTGCCTGGGCCCAAGAAAGCGCCCATGGATTC
CTTGTTCGACTACGGCACTTACCGTCACCACCCCAGTGACAACAAGAGAT
GGAGGAGAAAGGTCGTGGAGAAGCAGCCACAGAGCCCCAAAGCTCCTGCA
CCCCAGCCACCCCCCATCCTCAAAGTCTTCAATCGGCCCATCCTCTTTGA
CATTGTGTCCCGGGGCTCCACTGCGGACCTAGATGGACTGCTCTCCTTCT
TGTTGACCCACAAGAAGCGCCTGACTGATGAGGAGTTCCGGGAGCCGTCC
ACGGGGAAGACCTGCCTGCCCAAGGCGCTGCTGAACCTAAGCAACGGGCG
CAACGACACCATCCCGGTGTTGCTGGACATTGCGGAGCGCACCGGCAACA
TGCGTGAATTCATCAACTCGCCCTTCAGAGACATCTACTACCGAGGCCAG
ACATCCCTGCACATTGCCATCGAACGGCGCTGCAAGCACTACGTGGAGCT
GCTGGTGGCCCAGGGAGCCGACGTGCACGCCCAGGCCCGCGGCCGCTTCT
TCCAGCCCAAGGATGAGGGAGGCTACTTCTACTTTGGGGAGCTGCCCTTG
TCCCTGGCAGCCTGCACCAACCAGCCGCACATCGTCAACTACCTGACAGA
GAACCCTCACAAGAAAGCTGACATGAGGCGACAGGACTCGAGGGGGAACA
CGGTGCTGCACGCGCTGGTGGCCATCGCCGACAACACCCGAGAGAACACC
AAGTTTGTCACCAAGATGTACGACCTGCTGCTTCTCAAGTGTTCACGCCT
CTTCCCCGACAGCAACCTGGAGACAGTTCTCAACAATGATGGCCTTTCGC
CTCTCATGATGGCTGCCAAGACAGGGAAGATCGGGGTCTTTCAGCACATC
ATCCGACGTGAGGTGACAGATGAGGACACCCGGCATCTGTCTCGCAAGTT
CAAGGACTGGGCCTATGGGCCTGTGTATTCTTCTCTCTACGACCTCTCCT
CCCTGGACACATGCGGGGAGGAGGTGTCCGTGCTGGAGATCCTGGTGTAC
AACAGCAAGATCGAGAACCGCCATGAGATGCTGGCTGTAGAGCCCATTAA
CGAACTGTTGAGAGACAAGTGGCGTAAGTTTGGGGCTGTGTCCTTCTACA
TCAACGTGGTCTCCTATCTGTGTGCCATGGTCATCTTCACCCTCACCGCC
TACTATCAGCCACTGGAGGGCACGCCACCCTACCCTTACCGGACCACAGT
GGACTACCTGAGGCTGGCTGGCGAGGTCATCACGCTCTTCACAGGAGTCC
TGTTCTTCTTTACCAGTATCAAAGACTTGTTCACGAAGAAATGCCCTGGA
GTGAATTCTCTCTTCGTCGATGGCTCCTTCCAGTTACTCTACTTCATCTA
CTCTGTGCTGGTGGTTGTCTCTGCGGCGCTCTACCTGGCTGGGATCGAGG
CCTACCTGGCTGTGATGGTCTTTGCCCTGGTCCTGGGCTGGATGAATGCG
CTGTACTTCACGCGCGGGTTGAAGCTGACGGGGACCTACAGCATCATGAT
TCAGAAGATCCTCTTCAAAGACCTCTTCCGCTTCCTGCTTGTGTACCTGC
TCTTCATGATCGGCTATGCCTCAGCCCTGGTCACCCTCCTGAATCCGTGC
ACCAACATGAAGGTCTGTGACGAGGACCAGAGCAACTGCACGGTGCCCAC
GTATCCTGCGTGCCGCGACAGCGAGACCTTCAGCGCCTTCCTCCTGGACC
TCTTCAAGCTCACCATCGGCATGGGAGACCTGGAGATGCTGAGCAGCGCC
AAGTACCCCGTGGTCTTCATCGTCCTGCTGGTCACCTACATCATCCTCAC
CTTCGTGCTCCTGTTGAACATGCTTATCGCCCTCATGGGTGAGACCGTGG
GCCAGGTGTCCAAGGAGAGCAAGCACATCTGGAAGTTGCAGTGGGCCACC
ACCATCCTGGACATCGAGCGTTCCTTCCCTGTGTTCCTGAGGAAGGCCTT
CCGCTCCGGAGAGATGGTGACTGTGGGCAAGAGCTCAGATGGCACTCCGG
ACCGCAGGTGGTGCTTCAGGGTGGACGAGGTGAACTGGTCTCACTGGAAC
CAGAACTTGGGCATCATTAACGAGGACCCTGGCAAGAGTGAAATCTACCA
GTACTATGGCTTCTCCCACACCGTGGGGCGCCTTCGTAGGGATCGTTGGT
CCTCGGTGGTGCCCCGCGTAGTGGAGCTGAACAAGAACTCAAGCGCAGAT
GAAGTGGTGGTACCCCTGGATAACCTAGGGAACCCCAACTGTGACGGCCA
CCAGCAGGGCTACGCTCCCAAGTGGAGGACGGACGATGCCCCACTGTAGG
GGCCGTGCCAGAGCTCGCACAGATAGTCCAGGCTTGGCCTTCGCTCCCAC
CTACATTTAGGCATTTGTCCGGTGTCTTCCCACACCCGCATGGGACCTTG
GAGGTGAGGGCCTCTGTGGCGACTCTGTGGAGGCCCCAGGACCCTCTGGT
CCCCGCCAAGACTTTTGCCTTCAGCTCTACTCGCCACATGGGGGGGCGGG
GCTCCTGGCTACCTGTCTCGCTCGCTCCCATGGAGTCACCTAAGCCAGCA
CAAGGCCCCTCTCCTCGAAAGGCTCAGGCCCCATCCCTCTTGTGTATTAT
TTATTGCTCTCCTCAGGAAAATGGGGTGGCAGGAGTCCACCCGCGGCTGG
AACCTGGCCAGGGCTGAAGCTCATGCAGGGACGCTGCAGCTCCGACCTGC
CACAGATCTGACCTGCTGCAGCCCTGGCTAGTGTGGGTCTTCTGTACTTT
GAAGAGATCGGGGCCGCTGGTGCTCAATAAATGTTTATTCTCGGTGGAAA
AAAAAAAAAAAAAAAAAAAAAAAAAAAAAAAAAAAAAAAAAAAAAAAAAA
AAAAAAAAAAAAAAAAAAAAAAAA,
wherein R may be an A or G, M may be an A or C, S may be a C or G, Y may be a C or T, K may be a G or T and W may be an A or T.

19. Nucleic acid, characterised in that it contains the sequence
ATGGCAGATCCTGGTGATGGTCCCCGTGCAGCGCCTGGGGAGGTGGCTGA
GCCCCCTGGAGATGAGAGTGGTACCTCTGGTGGGGAGGCCTTCCCCCTCT
CTTCCCTGGCCAATCTGTTTGAGGGGGAGGAAGGCTCCTCTTCTCTTTCC
CCGGTGGATGCTAGCCGCCCTGCTGGCCCTGGCGATGGACGTCCAAACCT
GCGTATGAAGTTCCAGGGCGCTTTCCGCAAGGGGGTTCCCAACCCCATTG
ACCTGTTGGAGTCCACCCGGTACGAGTCGTCAGTAGTGCCTGGGCCCAAG
AAAGCGCCCATGGATTCCTTGTTCGACTACGGCACTTACCGTCACCACCC
CAGTGACAACAAGAGATGGAGGAGAAAGGTCGTGGAGAAGCAGCCACAGA
GCCCCAAAGCTCCTGCACCCCAGCCACGCCCCATCCTCAAAGTCTTCAAT
CGGCCCATCCTCTTTGACATTGTGTCCCGGGGCTCCACTGCGGACCTAGA
TGGACTGCTCTCCTTCTTGTTGACCCACAAGAAGCGCCTGACTGATGAGG
AGTTCCGGGAGCCGTCCACGGGGAAGACCTGCCTGCCCAAGGCGCTGCTG
AACCTAAGCAACGGGCGCAACGACACCATCCCGGTGTTGCTGGACATTGC
GGAGCGCACCGGCAACATGCGTGAATTCATCAACTCGCCCTTCAGAGACA
TCTACTACCGAGGCCAGACATCCCTGCACATTGCCATCGAACGGCGCTGC
AAGCACTACGTGGAGCTGCTGGTGGCCCAGGGAGCCGACGTGCACGCCCA
GGCCCGCGGCCGCTTCTTCCAGCCCAAGGATGAGGGAGGCTACTTCTACT
TTGGGGAGCTGCCCTTGTCCCTGGCAGCCTGCACCAACCAGCCGCACATC
GTCAACTACCTGACAGAGAACCCTCACAAGAAAGCTGACATGAGGCGACA
GGACTCGAGGGGGAACACGGTGCTGCACGCGCTGGTGGCCATCGCCGACA
ACACCCGAGAGAACACCAAGTTTGTCACCAAGATGTACGACCTGCTGCTT
CTCAAGTGTTCACGCCTCTTCCCCGACAGCAACCTGGAGACAGTTCTCAA
CAATGATGGCCTTTCGCCTCTCATGATGGCTGCCAAGACAGGCAAGATCG
GGGTCTTTCAGCACATCATCCGACGTGAGGTGACAGATGAGGACACCCGG
CATCTGTCTCGCAAGTTCAAGGACTGGGCCTATGGGCCTGTGTATTCTTC
TCTCTACGACCTCTCCTCCCTGGACACATGCGGGGAGGAGGTGTCCGTGC
TGGAGATCCTGGTGTACAACAGCAAGATCGAGAACCGCCATGAGATGCTG
GCTGTAGAGCCCATTAACGAACTGTTGAGAGACAAGTGGCGTAAGTTTGG
GGCTGTGTCCTTCTACATCAACGTGGTCTCCTATCTGTGTGCCATGGTCA
TCTTCACCCTCACCGCCTACTATCAGCCACTGGAGGGCACGCCACCCTAC
CCTTACCGGACCACAGTGGACTACCTGAGGCTGGCTGGCGAGGTCATCAC
GCTCTTCACAGGAGTCCTGTTCTTCTTTACCAGTATCAAAGACTTGTTCA
CGAAGAAATGCCCTGGAGTGAATTCTCTCTTCGTCGATGGCTCCTTCCAG
TTACTCTACTTCATCTACTCTGTGCTGGTGGTTGTCTCTGCGGCGCTCTA
CCTGGCTGGGATCGAGGCCTACCTGGCTGTGATGGTCTTTGCCCTGGTCC
TGGGCTGGATGAATGCGCTGTACTTCACGCGCGGGTTGAAGCTGACGGGG
ACCTACAGCATCATGATTCAGAAGATCCTCTTCAAAGACCTCTTCCGCTT
CCTGCTTGTGTACCTGCTCTTCATGATCGGCTATGCCTCAGCCCTGGTCA
CCCTCCTGAATCCGTGCACCAACATGAAGGTCTGTGACGAGGACCAGAGC
AACTGCACGGTGCCCACGTATCCTGCGTGCCGCGACAGCGAGACCTTCAG
CGCCTTCCTCCTGGACCTCTTCAAGCTCACCATCGGCATGGGAGACCTGG
AGATGCTGAGCAGCGCCAAGTACCCCGTGGTCTTCATCCTCCTGCTGGTC
ACCTACATCATCCTCACCTTCGTGCTCCTGTTGAACATGCTTATCGCCCT
CATGGGTGAGACCGTGGGCCAGGTGTCCAAGGAGAGCAAGCACATCTGGA
AGTTGCAGTGGGCCACCACCATCCTGGACATCGAGGGTTCCTTCCCTGTG
TTCCTGAGGAAGGCCTTCCGCTCCGGAGAGATGGTGACTGTGGGCAAGAG
CTCAGATGGCACTCCGGACCGCAGGTGGTGCTTCAGGGTGGACGAGGTGA
ACTGGTCTCACTGGAACCAGAACTTGGGCATCATTACGAGGACCCTGGCA
AGAGTGAAATCTACCAGTACTATGGCTTCTCCCACACCGTGGGGCGCCTT
CGTAGGGATCGTTGGTCCTCGGTGGTGCCCCGCGTAGTGGAGCTGAACPA
GPCTCAAGCGCAGATGAAGTGGTGGTACCCCTGGATAACCTAGGGAACCC
CAACTGTGACGGCCACCAGCAGGGCTACGCTCCCAAGTGGAGGACGGACG
ATGCCCCACTGTAG
or a partial sequence thereof, a nucleic acid which is capable of hybridising with said sequence under stringent conditions, an allelic variant or a functional variant of said sequence or a variant of the nucleic acid on the basis of the degenerative code.

20. Nucleic acid, characterised in that it has the sequence
ATGGCAGATCCTGGTGATGGTCCCCGTGCAGCGCCTGGGGAGGTGGCTGA
GCCCCCTGGAGATGAGAGTGGTACCTCTGGTGGGGAGGCCTTCCCCCTCT
CTTCCCTGGCCAATCTGTTTGAGGGGGAGGAAGGCTCCTCTTCTCTTTCC
CCGGTGGATGCTAGCCGCCCTGCTGGCCCTGGCGATGGACGTCCAAACCT
GCGTATGAAGTTCCAGGGCGCTTTCCGCAAGGGGGTTCCCAACCCCATTG
ACCTGTTGGAGTCCACCCGGTACGAGTCCTCAGTAGTGCCTGGGCCCAAG
AAAGCGCCCATGGATTCCTTGTTCGACTACGGCACTTACCGTCACCACCC
CAGTGACAACAAGAGATGGAGGAGAAAGGTCGTGGAGAAGCAGCCACAGA
GCCCCAAAGCTCCTGCACCCCAGCCACCCCCCATCCTCAAAGTCTTCAAT
CGGCCCATCCTCTTTGACATTGTGTCCCGGGGCTCCACTGCGGACCTAGA
TGGACTGCTCTCCTTCTTGTTGACCCACAAGAAGCGCCTGACTGATGAGG
AGTTCCGGGAGCCGTCCACGGGGAAGACCTGCCTGCCCAAGGCGCTGCTG
AACCTAAGCAACGGGCGCAACGACACCATCCCGGTGTTGCTGGACATTGC
GGAGCGCACCGGCAACATGCGTGAATTCATCAACTCGCCCTTCAGAGACA
TCTACTACCGAGGCCAGACATCCCTGCACATTGCCATCGAACGGCGCTGC
AAGCACTACGTGGAGCTGCTGGTGGCCCAGGGAGCCGACGTGCACGCCCA
GGCCCGCGGCCGCTTCTTCCAGCCCAAGGATGAGGGAGGCTACTTCTACT
TTGGGGAGCTGCCCTTGTCCCTGGCAGCCTGCACCAACCAGCCGCACATC
GTCAACTACCTGACAGAGAACCCTCACAAGAAAGCTGACATGAGGCGACA
GGACTCGAGGGGGAACACGGTGCTGCACGCGCTGGTGGCCATCGCCGACA
ACACCCGAGAGAACACCAAGTTTGTCACCAAGATGTACGACCTGCTGCTT
CTCAAGTGTTCACGCCTGTTCCCCGACAGCAACCTGGAGACAGTTCTCAA
CAATGATGGCCTTTCGCCTCTCATGATGGCTGCCAAGACAGGCAAGATCG
GGGTCTTTCAGCACATCATCCGACGTGAGGTGACAGATGAGGACACCCGG
CATCTGTCTCGCAAGTTCAAGGACTGGGCCTATGGGCCTGTGTATTCTTC
TCTCTACGACCTCTCCTCCCTGGACACATGCGGGGAGGAGGTGTCCGTGC
TGGAGATCCTGGTGTACAACAGCAAGATCGAGAACCGCCATGAGATGCTG
GCTGTAGAGCCCATTAACGAACTGTTGAGAGACAAGTGGCGTAAGTTTGG
GGCTGTGTCCTTCTACATCM&GTGGTCTCCTATCTGTGTGCCATGGTCAT
CTTCACCCTCACCGCCTACTATCAGCCACTGGAGGGCACGCCACCCTACC
CTTACCGGACCACAGTGGACTACCTGAGGCTGGCTGGCGAGGTCATCACG
CTCTTCACAGGAGTCCTGTTCTTCTTTACCAGTATCAAAGACTTGTTCAC
GAAGAAATGCCCTGGAGTGTTCTCTCTTCGTCGATGGCTCCTTCCAGTTA
CTCTACTTCATCTACTCTGTGCTGGTGGTTGTCTCTGCGGCGCTCTACCT
GGCTGGGATCGAGGCCTACCTGGCTGTGATGGTCTTTGCCCTGGTCCTGG
GCTGGATGAATGCGCTGTACTTCACGCGGGGGTTGAAGCTGACGGGGACC
TACAGCATCATGATTCAGAAGATCCTCTTCAAAGACCTCTTCCGCTTCCT
GCTTGTGTACCTGCTCTTCATGATCGGCTATGCCTCAGCCCTGGTCACCC
TCCTGAATCCGTGCACCAACATGAAGGTCTGTGACGAGGACCAGAGCAAC
TGCACGGTGCCCACGTATCCTGCGTGCCGCGACAGCGAGACCTTCAGCGC
CTTCCTCCTGGACCTCTTCAAGCTCACCATCGGCATGGGAGACCTGGAGA
TGCTGAGCAGCGCCAAGTACCCCGTGGTCTTCATCCTCCTGCTGGTCACC
TACATCATCCTCACCTTCGTGCTCCTGTTGAACATGCTTATCGCCCTCAT
GGGTGAGACCGTGGGCCAGGTGTCCAAGGAGAGCAAGCACATCTGGAAGT
TGCAGTGGGCCACCACCATCCTGGACATCGAGCGTTCCTTCCCTGTGTTC
CTGAGGAAGGCCTTCCGCTCCGGAGAGATGGTGACTGTGGGCAAGAGCTC
AGATGGCACTCCGGACCGCAGGTGGTGCTTCAGGGTGGACGAGGTGAACT
GGTCTCACTGGAACCAGAACTTGGGCATCATTAACGAGGACCCTGGCAAG
AGTGAAATCTACCAGTACTATGGCTTCTCCCACACCGTGGGGCGCCTTCG
TAGGGATCGTTGGTCCTCGGTGGTGCCCCGCGTAGTGGAGCTGAACAAGA
ACTCAAGCGCAGATGAAGTGGTGGTACCCCTGGATAACCTAGGGAACCCC
AACTGTGACGGCCACCAGCAGGGCTACGCTCCCAAGTGGAGGACGGACGA
TGCCCCACTGTAG.

21. Recombinant vector, characterised in that it contains a nucleic acid according to one of claims 1 to 20.

22. Recombinant vector according to claim 21, characterised in that it is an expression vector.

23. Host, characterised in that it contains a vector according to claim 21 or 22.

24. Host according to claim 23, characterised in that it is a eukaryotic host cell.

25. Host according to claim 23 or 24, characterised in that it is an insect cell.

26. Host according to one of claims 23 to 25, characterised in that it is an Sf9-, HEK293- or HeLa-cell.

27. Host according to claim 23, characterised in that it is a bacteriophage.

28. Host according to claim 23, characterised in that it is a prokaryotic host cell.

29. Polypeptide, characterised in that it is coded by a nucleic acid according to one of claims 1 to 20 or a fragment, a functional variant, an allelic variant, a subunit, a variant on the basis of the degenerative nucleic acid code, a chemical derivative thereof, a fusion protein with said polypeptide or a glycosylation variant thereof.

30. Polypeptide according to claim 29, characterised in that it is a fragment of the nonselective cation channel OTRPC4.

31. Polypeptide according to one of claims 29 and 30, characterised in that it is a functional variant of the nonselective cation channel OTRPC4.

32. Polypeptide according to one of claims 29 to 31, characterised in that it is an allelic variant of the nonselective cation channel OTRPC4.

33. Polypeptide according to one of claims 29 to 32, characterised in that it is a subunit of the nonselective cation channel OTRPC4.

34. Polypeptide according to one of claims 29 to 33, characterised in that it is a variant of the nonselective cation channel OTRPC4 on the basis of the degenerative nucleic acid code.

35. Polypeptide according to one of claims 29 to 34, characterised in that it is a chemical derivative of the nonselective cation channel OTRPC4.

36. Polypeptide according to one of claims 29 to 35, characterised in that it is a fusion protein consisting of the nonselective cation channel OTRPC4 and another protein.

37. Polypeptide according to one of claims 29 to 36, characterised in that it is a glycosylation variant of the nonselective cation channel OTRPC4.

38. Process for preparing polypeptides according to one of claims 29 to 37, characterised in that a host according to one of claims 23 to 28 is cultivated and said polypeptide is isolated.

39. Antibody protein, characterised in that it is specific for a polypeptide according to one of claims 29 to 37.

40. Process for preparing an antibody protein according to claim 39, characterised in that it comprises the following steps: a host selected from a eukaryotic or prokaryotic cell which contains one or more vectors having one or more nucleic acids specific for the antibody protein, is cultivated under conditions under which said antibody protein is expressed by said host cell and said antibody protein is isolated.

41. Use of a polypeptide according to one of claims 29 to 37 for finding blockers, activators or modulators of said polypeptides.

42. Use of a host according to one of claims 23 to 28 for finding blockers, activators or modulators of OTRPC4 channels.

43. Process for finding blockers, activators or modulators of OTRPC4, characterised in that a host according to one of claims 23 to 28 is incubated with a test substance.

44. Process according to claim 43, characterised in that a membrane current is measured, said membrane current is compared with a membrane current which is measured in said host after incubation with a known control substance or in the absence of the test substance.

45. Process according to one of claims 43 and 44, characterised in that said blocker is bound to a channel, said host is incubated with a test substance and the displacement of the blocker or activator bound to the channel by the test substance is measured.

46. Process according to one of claims 43 to 45, characterised in that a host according to one of claims 23 to 28 is incubated with a test substance, the intracellular quantity of a divalent cation is determined and said quantity of divalent cation is compared with the quantity of said divalent cation which is measured when said host is incubated with a known control or in the absence of the test substance.

47. Process according to one of claims 43 to 46, characterised in that said process is a high throughput screening (HTS) test or an ultrahigh throughput screening ( UHTS) test.

48. Activator of OTRPC4 which can be found using a process according to claims 43 to 47.

49. Blocker of OTRPC4 which can be found using a process according to claims 43 to 47.

50. Modulator of OTRPC4 which can be found using a process according to claims 43 to 47.

51. Antisense nucleic acid, characterised in that it is capable of hybridising with part of a nucleic acid according to one of claims 1 to 20 under stringent conditions.

52. Antisense nucleic acid according to claim 51, characterised in that it is a ribozyme.

53. Pharmaceutical composition, characterised in that it contains a nucleic acid according to one of claims 1 to 20 together with pharmaceutically acceptable carriers or excipients.

54. Pharmaceutical composition, characterised in that it contains an antisense nucleic acid according to one of claims 51 to 52 together with pharmaceutically acceptable carriers or excipients.

55. Pharmaceutical composition, characterised in that it contains a polypeptide according to one of claims 29 to 37 together with pharmaceutically acceptable carriers or excipients.

56. Pharmaceutical composition, characterised in that it contains a vector according to one of claims 21 to 22 together with pharmaceutically acceptable carriers or excipients.

57. Pharmaceutical composition, characterised in that it contains a host according to one of claims 23 to 28 together with pharmaceutically acceptable carriers or excipients.

58. Use of a nucleic acid according to one of claims 1 to 20 for preparing a medicament for the treatment of a disease selected from among diabetes, hyperlipidaemia, hyperproteinaemia, hypertension, stroke, renal insufficiency, shock and other pathophysiological conditions characterised by hyper- and hypoosmolarity.

59. Use of an antisense nucleic acid according to one of claims 51 to 52 for preparing a medicament for the treatment of a disease selected from among diabetes, hyperlipidaemia, hyperproteinaemia, hypertension, stroke, renal insufficiency, shock and other pathophysiological conditions charasterised by hyper- and hypoosmolarity.

60. Use of a vector according to one of claims 21 to 22 for preparing a medicament for the treatment of a disease selected from among diabetes, hyperlipidaemia, hyperproteinaemia, hypertension, stroke, renal insufficiency, shock and other pathophysiological conditions characterised by hyper- and hypoosmolarity.

61. Use of a host according to one of claims 23 to 28 for preparing a medicament for the treatment of a disease selected from among diabetes, hyperlipidaemia, hyperproteinaemia, hypertension, stroke, renal insufficiency, shock and other pathophysiological conditions characterised by hyper- and hypoosmolarity.

62. Non-human mammal, characterised in that, in addition to its genome, it contains a nucleic acid according to one of claims 1 to 20 (transgene).

63. Non-human mammal, characterised in that, in its genome, a nucleic acid according to one of claims 1 to 20 is inactivated (gene knock-out).

64. Non-human mammal, characterised in that, in its genome, a nucleic acid according to one of claims 1 to 20 is modified (gene knock-in).

65. Process for producing a non-human mammal, characterised in that
a) embryonic stem cells of said non-human mammal are transfected with a vector which contains a nucleic acid according to one of claims 1 to 20 and permits recombination between the genomic DNA of said non-human mammal and the nucleic acid contained in the vector
b) stably transfected stem cells from step a) are isolated and these are transferred into the germline of a female animal of said non-human mammal
c) the offspring of said female animal from step b) with a male animal of the same species are analysed for animals which express the polypeptide coded by the nucleic acid from step a).

66. Process for producing a non-human mammal, characterised in that
d) embryonic stem cells of said non-human mammal are transfected with a vector which contains a nucleic acid which is capable of hybridising with a nucleic acid according to one of claims 1 to 20 under stringent conditions and is inactivated by insertion of an additional nucleic acid sequence and permits recombination between the genomic DNA of said non-human mammal and the nucleic acid contained in the vector
e) stably transfected stem cells from step d) are isolated and these are transferred into the germline of a female animal of said non-human mammal
f) the offspring of said female animal from step e) with a male animal of the same species are analysed for animals which express the polypeptide coded by the nucleic acid from step d) only slightly or not at all.

67. Process for producing a non-human mammal, characterised in that
g) embryonic stem cells of said non-human mammal are transfected with a vector which contains a nucleic acid which is capable of hybridising with a nucleic acid according to one of claims 1 to 20 under stringent conditions and is modified by insertion of an additional nucleic acid sequence and permits recombination between the genomic DNA of said non-human mammal and the nucleic acid contained in the vector
h) stably transfected stem cells from step g) are isolated and these are transferred into the germline of a female animal of said non-human mammal
i) the offspring of said female animal from step h) with a male animal of the same species are analysed for animals which express the polypeptide coded by the nucleic acid from step g).

68. Use of a modulator, activator, or inhibitor of OTRPC4 for the regulation of the osmolarity of body fluids.

69. Use according to claim 68, wherein the body fluid is cerebrospinal fluid, intraocular fluid, or saliva.

70. Pharmaceuticall composition comprising a modulator, activator, or inhibitor of OTRPC4, and a pharmaceutically acceptable carrier, excipient, or diluent.
